(19) United States
(12) Patent Application Publication
(10) Pub. No.: US 2012/0121679 A1
(43) Pub. Date: May 17, 2012

(54) VIRICIDAL AND MICROBICIDAL COMPOSITIONS AND USES THEREOF

(75) Inventors: Jennifer L. Cannon, Griffin, GA (US); Michael Patrick Doyle, Peachtree City, GA (US); Tong Zhao, Peachtree City, GA (US)

(73) Assignee: University of Georgia Research Foundation, Inc., Athens, GA (US)

(21) Appl. No.: 13/350,315

(22) Filed: Jan. 13, 2012

Related U.S. Application Data
(63) Continuation-in-part of application No. PCT/US2010/042239, filed on Jul. 16, 2010.
(60) Provisional application No. 61/226,093, filed on Jul. 16, 2009.
(60) Provisional application No. 61/313,894, filed on Mar. 15, 2010.
(60) Provisional application No. 61/445,686, filed on Feb. 23, 2011.

(51) Int. Cl. A01N 25/34; A01N 25/08; A61K 31/19; A01P 1/00; A01P 3/00; A61P 31/22; A61P 31/10; A61P 31/04; A61P 31/14; A61P 31/16; A61P 31/20; A01N 37/02; A61P 31/12

(52) U.S. Cl. 424/404; 514/557; 424/402; 424/411; 424/409; 424/400

(57) ABSTRACT

The present disclosure encompasses compositions comprising a surfactant, and an acid such as, but not limited to, levulinic acid, that together have a synergistic effect in reducing the viability of a virus population compared to the efficacy of the individual compounds. This synergy allows the formulation of compositions where the active agents (including an acid and a surfactant) are present at concentrations effective to inactivate viruses on surfaces, including human skin. The viricidal compositions disclosed herein are efficacious without damaging the surface to which they may be applied, or even altering the organoleptic properties of a treated food substance. The viricidal compositions and wipes containing such compositions are suitable for sanitizing any surface suspected of having a viral load thereon, or where it is desirable to ensure that a viral load is as low as possible.

### CROSS-REFERENCE TO RELATED APPLICATIONS

This application is a U.S. Continuation-in-Part of application Serial No.: PCT/US/2010/042239, entitled “VIRICIDAL COMPOSITION AND USE” filed Jul. 16, 2010, which claims priority from U.S. Provisional Patent Application Ser. No. 61/226,093, entitled “VIRICIDAL COMPOSITION AND USE” filed on Jul. 16, 2009, and to U.S. Provisional Patent Application Ser. No. 61/313,894, entitled “SANITIZING WIPE” filed on Mar. 15, 2010, and which further claims priority from U.S. Provisional Patent Application Ser. No. 61/445,686, entitled “SKIN CLEANSER” filed on Feb. 23, 2010, the entireties of which are hereby incorporated by reference.

### TECHNICAL FIELD

The present disclosure is generally related to microbicidal compositions and particularly compositions having viricidal activity and to methods of use thereof.

### BACKGROUND

Many human diseases are caused by viruses that may be divided into two groups: enveloped and non-enveloped. “Enveloped” or “lipophilic” viruses have an outer lipid-based membrane enveloping the capsid (comprised solely of capsomere proteins) that in turn protects the innermost viral genetic material. The enveloping membrane contains both viral and host cell proteins, and is acquired during budding from the host cell at the end of the viral replication process. Enveloped viruses include respiratory syncytial virus (RSV), and coronavirus, as well as influenza, measles, Hepatitis B and C and Herpes simplex viruses.

Non-enveloped viruses do not have an enveloping membrane; their outer surface is the protein capsid. Such viruses include caliciviruses (norovirus and sapovirus), astrovirus, rhinovirus, rotavirus, adenovirus, Hepatitis E virus and Hepatitis A virus. Non-enveloped viruses may be less susceptible to conventional viricides than enveloped viruses. Typical antimicrobial agents that affect cell membranes, such as alcohol, may also affect the outer membrane of an enveloped virus, but as a sole active agent may have little or no effect on the capsids of either virus type, either enveloped or non-enveloped. Macinga et al., ((2008) Appl. Environ. Microbiol. 74: 5047-5052) describes a hand sanitizer comprised of individually inert ingredients that behaved synergistically to inactivate the human norovirus surrogate, murine norovirus (MNV-1) when combined. This chemical blend of 70% ethanol, polyquaternium-37, and citric acid yielded a 3.68-log reduction in PFU/ml of MNV-1 in solution and a 2.48-log PFU/ml reduction from fingerpads. Similarly, a study (Predmore & Li (2011) Appl. Environ. Microbiol. 77: 4829-4838) revealed enhanced removal and inactivation of MNV-1 on produce after treatment with a combination of surfactants, including SDS, and 200 ppm chlorine.

Non-enveloped viruses are particularly difficult to adequately disinfect from environmental surfaces. Strong oxidizers like peracetic acids and bleaches inactivate most viruses with sufficient time, concentration, and no organic load, but they cannot be used on many surfaces without damaging them. Traditional disinfectants based on quaternary ammonium compounds (QACs) as the sole active agent may also have little or no effect on such viruses.

Norovirus, a member of the family Caliciviridae, is one of the most difficult viruses to disinfect. Human noroviruses (NoV) have emerged globally as the leading cause of non-bacterial gastroenteritis and the second most frequent agent of severe childhood gastroenteritis. While the majority of outbreaks in hospitals, nursing homes, daycares, cruise ships, and schools are the result of person-to-person transmission, NoV is also the leading cause of outbreaks of food borne gastroenteritis, causing an estimated 30-50% of all food borne outbreaks in the United States. Of produce-related outbreaks involving greens-based salads, lettuce, and fruits, 67%, 47% and 67% were attributed to noroviruses, respectively, in the US in 1990-2005, exceeding the contribution of bacterial food-borne pathogens. Foodhandler contamination of ready-to-eat foods is of particular concern because human noroviruses can be shed in feces for days to weeks after symptoms have subsided, and even in the complete absence of symptomatic infection. An infectious norovirus dose can be as small as 1-10 viral units so that even low contamination levels can jeopardize food safety. In addition, noroviruses are environmentally robust, surviving on surfaces for several days to more than a week, and recent data indicate washing with chlorinated water, at concentrations typically used on food and food preparation surfaces, may be inadequate to remove and inactivate high levels of contamination of noroviruses on fruit such as raspberries.

To date, there is no reliable cultivation assay for human norovirus. Thus all studies addressing norovirus survival and inactivation have relied on physically similar and genetically related surrogate viruses, such as Murine Norovirus (MNV) or Feline Calicivirus (FCV). Since its discovery and cultivation in 2003, MNV has often proven to be a more robust and reliable surrogate for human NoV than FCV when low pH sanitizers are being evaluated.

Escherichia coli O157:H7 and Salmonella are major causes of severe food borne disease in the United States and continue to be of public health significance. Salmonella is one of the most frequent causes of food borne illnesses worldwide. In the United States, it causes an estimated 1.4 million cases of illness, approximately 20,000 hospitalizations, and more than 500 deaths annually (Mead, et al., 1999). FoodNet surveillance data of food borne illnesses revealed that the overall incidence of salmonellosis has decreased by only 8% from 1996-1998 to 2004 and the incidence of Salmonella enteritidis infections has remained at approximately the same level.

Other pathogens such as, for instance, Klebsiela, V. cholera, Proteus hauseri, Shigella, Yersinia pestis and B. anthracis, and protozoan parasites, together with the more prominent E. coli and Salmonella, comprise a wide-spectrum of food-borne and water-borne pathogens which threatens the safety of the food supply and are now considered a matter of homeland security relevance. These food-borne and water-borne microorganisms are also associated with the spoilage of beverages such as fruit juices, and other protein and/or sugar-containing beverages. Therefore, the development of a unique, pluripotent, widely applicable, and easy to manufacture countermeasure is desirable.

There is growing interest in the development of novel antimicrobial treatments such as combinations of natural antimicrobials, including generally recognized as safe (GRAS) chemicals. Such compositions have also been shown to be effective against a large spectrum of food borne pathogens, leading to the reduction of pathogen populations by factors often greater than 7 log. Pharmaceutically acceptable chemical compositions have been formulated and have been demonstrated as effective in killing Salmonella on chicken skin and in chicken-processing water, and both Salmonella and E. coli O157:H7 on fresh produce without producing any detectable impact on the organoleptic properties of the treated food.

The efficacy of levulinic acid plus SDS at different concentrations and ratios in inactivating spores of Alicyclobacillus and Bacillus species was demonstrated in liquid preparations and with several isolates of each genus tested individually. Inactivation was also demonstrated for spore preparations of these bacteria after treatment with levulinic acid plus SDS in combination with a heat treatment of 65° C. for 30 min.

### SUMMARY

The present disclosure encompasses compositions comprising surfactants and an acid, particularly, but not limited to, levulinic acid that has a synergistic effect in reducing the viability of a virus population compared to the efficacy of the individual compounds. This synergy allows the formulation of compositions where the active agents (including an acid and a surfactant) are present at concentrations effective to inactivate viruses on surfaces, including human skin. The viricidal compositions disclosed herein are efficacious without damaging the surface to which they may be applied, or even altering the organoleptic properties of a treated food substance. The viricidal compositions and the wipes containing such compositions are suitable for sanitizing any surface suspected of having a viral load thereon or where it is desirable to ensure that a viral load is as low as possible.

One aspect of the disclosure encompasses embodiments of an antimicrobial composition comprising: a monoprotic organic acid comprising a carbon backbone of 3 to 13 carbons having the general structure of:

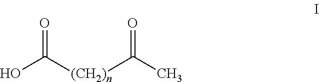

where n is an integer selected from 1 to 10, and where the concentration of the acid in said composition can be about 0.2% to about 20% by weight per volume of solvent; a surfactant, having a concentration about 0.05% to about 5% by weight per volume of solvent; and an aqueous solvent, where the antimicrobial composition is formulated to be effective in reducing the viability of a viral population, a bacterial population, a fungal population, or of any combination thereof.

In embodiments of this aspect of the disclosure, the antimicrobial composition can be formulated to be effective in reducing the viability of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the surfactant can be an anionic surfactant selected from the group consisting of: sodium dodecyl sulfate, sodium laureth sulfate, cetylpyridinium chloride, cetylpyridinium bromide, and benzalkonium chloride.

In embodiments of this aspect of the disclosure, the antimicrobial composition can further comprise a gelling agent, a foaming agent, a soap, a colorant, a fragrance, or any combination thereof.

In embodiments of this aspect of the disclosure, the antimicrobial composition can be formulated as a liquid; a foam having a cylinder foam test half-life of at least ten minutes, or a mix precursor thereof; a gel; or a solid or semi-solid soap.

In embodiments of this aspect of the disclosure, the solvent can be water or an alcohol:water, where the alcohol can be selected from the group consisting of ethanol, propanol, isopropanol, butanol, propylene glycol, diethylene glycol, dipropylene glycol, or any mixture thereof.

In embodiments of this aspect of the disclosure, the antimicrobial composition can further comprise a cationic agent selected from the group consisting of: benzalkonium chloride, benzethonium chloride, triclocarban, tricolsan, chlorhexidine, and any combination thereof.

In the embodiments of this aspect of the disclosure, the composition can be selected from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; about 5% levulinic acid and about 2% sodium dodecyl sulfate by weight per volume solvent.

In embodiments of this aspect of the disclosure, the antimicrobial composition can be deposited on or within a flexible support material.

In these embodiments of this aspect of the disclosure, the flexible support material can be a cloth, a fabric, a paper, a natural fiber mesh, a synthetic fiber mesh, a combination natural and synthetic fiber mesh, a brush-like surface, or a porous fabric.

In embodiments of this aspect of the disclosure, the antimicrobial composition can be substantially free of a solvent, wherein the pharmaceutically acceptable surfactant and the monoprotic organic acid are in a weight ratio of between about 1:200 to about 16.6:1.

Another aspect of the disclosure encompasses embodiments of sanitizing wipe comprising a flexible support material and an antimicrobial composition absorbed thereon, where the antimicrobial composition can comprise levulinic acid, sodium dodecyl sulfate, and a solvent, where the total concentration of the levulinic acid is about 0.2% to about 20% by weight per volume of solvent and the total concentration of the sodium dodecyl sulfate is about 0.05% to about 5% by weight per volume of solvent, and where the antimicrobial composition can be formulated to be effective in reducing the viability of a microbial population.

In embodiments of this aspect of the disclosure, the antimicrobial composition can be formulated to be effective in reducing the viability of a viral population, a bacterial population, a fungal population, or of any combination thereof.

In some embodiments of this aspect of the disclosure, the antimicrobial composition can formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the flexible support material can have a surface-positive charge thereon.

In embodiments of this aspect of the disclosure, the solvent can be water or an alcohol:water mix, where the alcohol can be selected from the group consisting of ethanol, propanol, isopropanol, butanol, propylene glycol, diethylene glycol, dipropylene glycol, or any mixture thereof.

In embodiments of this aspect of the disclosure, the composition can further comprise a cationic agent selected from the group consisting of: benzalkonium chloride, benzethonium chloride, triclocarban, tricolsan, chlorhexidine, and any combination thereof.

In embodiments of this aspect of the disclosure, the can be selected from the group consisting of about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; and about 5% levulinic acid by weight per volume solvent and about 2% sodium dodecyl sulfate by weight per volume solvent.

In embodiments of this aspect of the disclosure, the composition can further comprise a gelling agent, a foaming agent, a soap, a colorant, a fragrance, or any combination thereof.

In embodiments of this aspect of the disclosure, the flexible support material can be a cloth, a fabric, a paper, a natural fiber mesh, a synthetic fiber mesh, a combination natural and synthetic fiber mesh, a brush-like surface, or a porous fabric.

Yet another aspect of the disclosure encompasses embodiments of a method of reducing the viability of a microbial population, the method comprising contacting a microbial population with an antimicrobial composition comprising about 0.2% to about 20% by weight of levulinic acid per volume of solvent, about 0.05% to about 5% by weight of sodium dodecyl sulfate per volume of solvent, and an aqueous solvent, whereby the viability of the population of viruses is reduced.

In embodiments of this aspect of the disclosure, the microbial population can be on a non-liquid surface. In other embodiments of this aspect of the disclosure, the microbial population can be on a skin surface.

In embodiments of this aspect of the disclosure, the antimicrobial composition can be formulated to be effective in reducing the viability of a viral population, a bacterial population, a fungal population, or of any combination thereof.

In embodiments of this aspect of the disclosure, the antimicrobial composition can be formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the composition can be selected from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume; and about 5% levulinic acid by weight per volume solvent and about 2% sodium dodecyl sulfate by weight per volume solvent.

In embodiments of this aspect of the disclosure, the composition can be disposed on a flexible support material. In some embodiments of this aspect of the disclosure, the flexible support material can include a positive ionic charge thereon.

In embodiments of this aspect of the disclosure, the antimicrobial composition can be applied to a viral population is formulated as a liquid wash, a spray, a foam, a paste, a cream, a gel, or a wipe.

In embodiments of this aspect of the disclosure, the antimicrobial composition can be formulated to be effective in reducing the viability of a microbial population on a skin surface, where the microbial population is a viral population, a bacterial population, a fungal population, or any combination thereof, and wherein the antimicrobial composition is applied to the microbial population as a liquid wash, a spray, a foam, a paste, a cream, a gel, or a wipe.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIG. 1 is a graph showing the log reduction in plaque forming units (PFU/ml) of MNV dried on stainless steel coupons after treatment with dry wipes of various surface charges using 1 or 5 wiping motions. Error bars indicate standard deviation.

FIG. 2 is a graph showing the log reduction in PFU/ml of Murine Norovirus (MNV) dried on stainless steel coupons after treatment with wet wipes of various surface charges in combination with levulinic acid plus sodium dodecyl sulfate and compared to water using 1 or 5 wiping motions. Error bars indicate standard deviation.

FIG. 3 is a graph showing the log reduction in PFU/ml of Hepatitis A virus (HAV) dried on stainless steel coupons after treatment with dry wipes of various surface charges using 1 or 5 wiping motions. Error bars indicate standard deviation.

FIG. 4 is a graph showing the log reduction in PFU/ml of HAV dried on stainless steel coupons after treatment with wet wipes of various surface charges in combination with levulinic acid plus sodium dodecyl sulfate and compared to water using 5 wiping motions. Error bars indicate standard deviation.

FIG. 5 is a graph showing the log reduction in CFU/ml of Salmonella enterica dried on stainless steel coupons after treatment with dry wipes of various surface charges using 1 or 5 wiping motions. Error bars indicate standard deviation.

FIG. 6 is a graph showing the log reduction in CFU/ml of Salmonella enterica dried on stainless steel coupons after treatment with wet wipes of various surface charges in combination with levulinic acid plus sodium dodecyl sulfate and compared to water using 1 or 5 wiping motions. Error bars indicate standard deviation.

FIG. 7 is a graph showing the log reduction in PFU/ml of MNV dried on latex gloves after treatment with wet wipes of positive and neutral charge in combination with levulinic acid plus sodium dodecyl sulfate and compared to water using 1 or 5 wiping motions. Error bars indicate standard deviation.

FIG. 8 is a graph showing the log reduction in PFU/ml of MNV on stainless steel with 5% levulinic acid plus 2% sodium dodecyl sulfate as a liquid or as a foaming treatment with varying concentrations of FBS in the inoculum (n=5).

FIG. 9 is a graph showing the log reduction in PFU/ml of MNV on the surface of grapes after treatment with 5% levulinic acid plus 2% sodium dodecyl sulfate or water for 1 min or 5 mins.

FIG. 10 is a graph illustrating the log reduction in PFU/ml of viable MNV-1 after treatment with 5% levulinic acid plus 2% SDS solution at ambient pH (2.8) or after pH adjustment (to pH 4 or 4.5) for 1 min at 21° C. Error bars indicate standard deviations.

FIG. 11 is a graph illustrating the log reduction in PFU/ml of viable MNV-1 on stainless steel surfaces after treatment with sterile water or 5% levulinic acid plus 2% SDS in a liquid solution or as a foaming treatment for 1 min at 21° C. The amount of organic material (FBS) added to the virus stock was varied from 0 to 10% as indicated. Error bars indicate standard deviations.

FIG. 12 is a graph illustrating the log reduction in PFU/ml of viable MNV-1 on stainless steel surfaces after treatment with sterile water or 5% levulinic acid plus 2% SDS in a liquid solution or as a foaming treatment for 5 min at 21° C. The amount of organic material (FBS) added to the virus stock was varied from 0-10% as indicated. Error bars indicate standard deviations.

FIGS. 13A-13E illustrate bar graphs demonstrating the efficacy of levulinic acid and SDS, alone or in combination, to kill spores of Bacillus anthracis Sterne. Spores were exposed to one of six different solutions: A: 3% levulinic acid plus 2% SDS; B: 2% levulinic acid plus 1% SDS; C: 0.5% levulinic acid plus 0.05% SDS; D: 3% levulinic acid; E: 2% SDS; or F: water (serving as the control) for various lengths of time before testing the spores for viability relative to the control sample. Average plate counts are based on counting three plates; error bars indicate +/−one standard deviation.

Period of exposure: FIG. 13A, 0 min; FIG. 13B, 10 min; FIG. 13C, 45 min; FIG. 13D, 90 min; FIG. 13E, 180 min.

FIGS. 14A-14E illustrate bar graphs demonstrating the efficacy of levulinic acid and SDS, alone or in combination, to kill spores of Bacillus anthracis Sterne. Spores were exposed to one of six different solutions: A: 3% levulinic acid plus 2% SDS; B: 2% levulinic acid plus 1% SDS; C: 0.5% levulinic acid plus 0.05% SDS; D: 3% levulinic acid; E: 2% SDS; and F: water (serving as the control) for time intervals before testing the spores for viability relative to the control sample. In order to differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts are based on counting three plates; error bars indicate +/−one standard deviation.

Period of exposure: FIG. 14A, 0 hr; FIG. 14B, 1 hr; FIG. 14C, 2 hrs; FIG. 14D, 3 hrs; FIG. 14E, 4 hrs.

FIGS. 15A-15E represent bar graphs demonstrating the efficacy of levulinic acid and SDS, alone or in combination, to kill spores of Bacillus anthracis Sterne. Spores were exposed to one of six different solutions: A: 3% levulinic acid plus 2% SDS; B: 2% levulinic acid plus 1% SDS; C: 0.5% levulinic acid plus 0.05% SDS; D: 3% levulinic acid; E: 2% SDS; and F: water (serving as the control) for time intervals before testing the spores for viability relative to the control sample. To differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts are based on counting three plates; error bars indicate +/−one standard deviation.

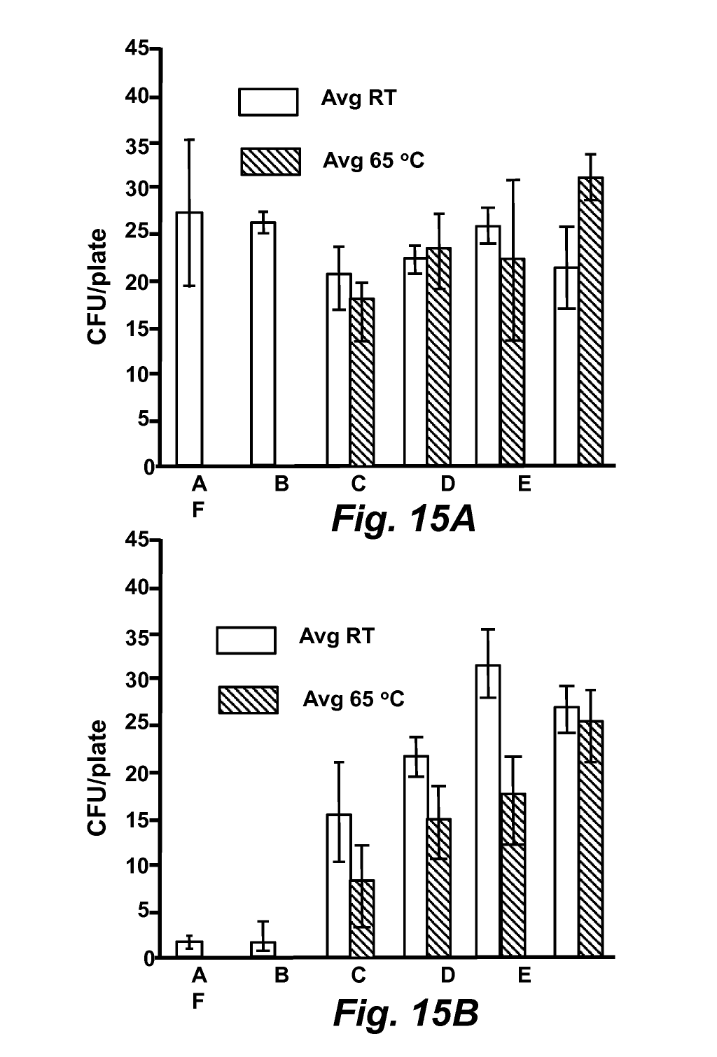

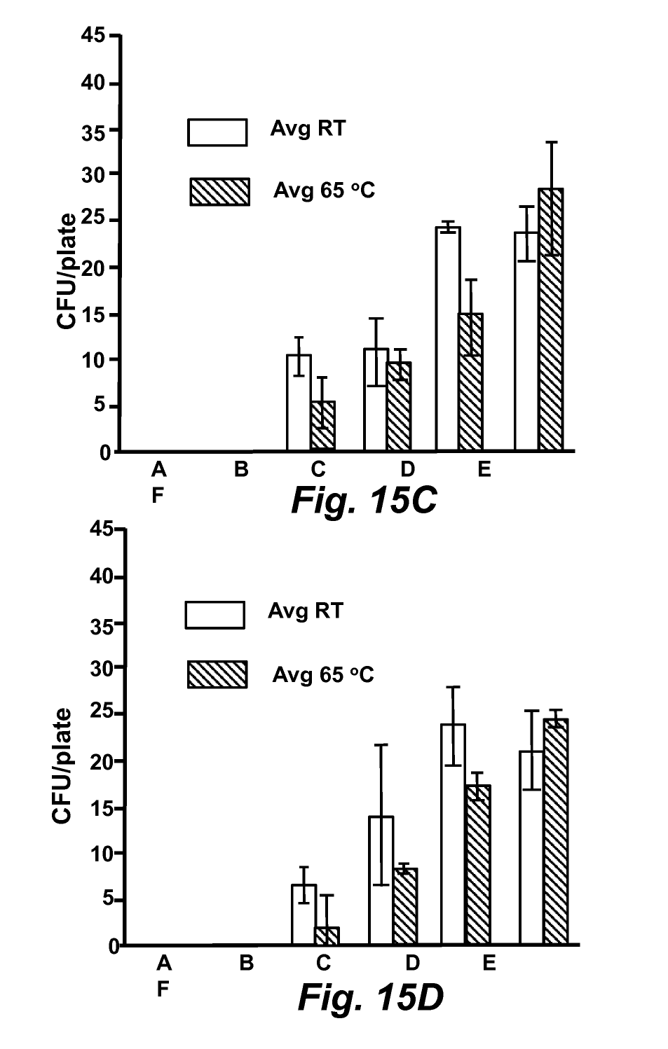

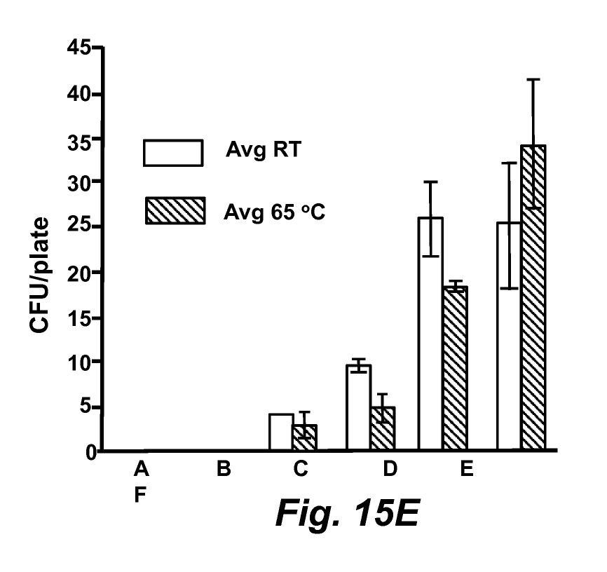

Period of exposure: FIG. 15A, 0 hr; FIG. 15B, 1 hr; FIG. 15C, 2 hrs; FIG. 15D, 3 hrs; FIG. 15E, 4 hrs.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “comprising” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Comprising” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### DEFINITIONS

In describing and claiming the invention, the following terminology will be used in accordance with the definitions set forth below.

The terms “antimicrobial”, “antiviral” and the term “viricide” as used herein are intended to include any compound or composition that inactivates or decreases the ability of a virus to infect a cell and/or replicate. Typically an effective antiviral or viricide will reduce the viral infectivity by at least a 2-5 log factor for a single application of the compound, although a 7 log factor can also be contemplated. Higher levels of reduction in viral infectivity may be achieved by repeat application of the compound or if used in conjunction with other cleansing or sanitizing agents. It is contemplated that an “antimicrobial” as herein referred may include, in addition to an antiviral activity other antimicrobial activity including, but not limited to, an antibacterial activity.

The term “acid” or “organic acid” as used herein refers to a compound having a hydrocarbon chain and an acid group covalently bound to the hydrocarbon chain. The hydrocarbon chain can be of any length and can be a straight chain or branched chain. The most common organic acids are the carboxylic acids whose acidity is associated with their carboxyl group —COOH. However, additional compounds that lack a carboxylic function group can still function as an acid in accordance with the present invention if the compound ionizes in aqueous solution to yield hydrogen ions. Accordingly, eugenol is considered an acid within the context of the present invention due to the electron withdrawing properties of the phenol ring on the hydroxyl group substitutent. Sulfonic acids, containing the group —OSO₃H, are another typical, but relatively stronger group of organic acids. In accordance with one embodiment the organic acid is a carboxylic acid comprising a maximum of 3 to 8 carbon atoms. The organic acids used in the embodiments of the present disclosure may also include additional functional groups extending from the hydrocarbon backbone. The carbon chain of the organic acid is functionalized by a hydroxyl, a carbonyl, an amino, an alkylamino, a sulfonyl, or a thiol group.

A monoprotic acid is an acid that is able to donate one proton per molecule during ionization.

A quaternary ammonium cation is a compound of the general structure:

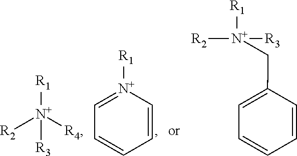

where R₁, R₂, R₃, and R₄ are independently selected from the group consisting of C₁-C₂₀ alkyl and salts thereof.

As used herein the term “benzalkonium chloride” refers to a single alkylbenzyldimethylammonium chloride of the general structure:

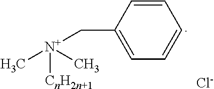

wherein n is an integer selected from the group consisting of 6, 8, 10, 12, 14, 16, 18 and 20, or mixtures of two or more such compounds.

The terms “effective” and “effective amount” as used herein refers to a concentration of active agent of an anti-microbial composition that provides the desired effect, i.e., log orders of reduction in surface microbial counts on a surface, including the surface of foodstuffs without reducing organoleptic properties of the food substance.

The term “surface” as used herein refers to a surface that is desired to be sanitized such as, but not limited to, a glove (latex or non-latex) including surgical gloves, a tool, a surgical tool or apparatus, a machine, equipment, a structure, a building, play materials, bathroom interiors, or other household surfaces, or the like, or the skin surface of an animal or human. Examples of food processing surfaces include surfaces of food processing or preparation equipment (e.g., slicing, canning, or transport equipment, including flumes), of food processing wares (e.g., utensils, dishware, washware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing, blancher cleaning, food packaging materials, cutting boards, beverage chillers and warmers, meat chilling or scalding equipment, cooling towers, food processing garment areas (including drains). Play material surfaces include, but are not limited to, surfaces of toy articles, playground equipment, cards and poker chips. Bathroom surfaces include such as sinks, toilets, walls, door handles, and fixtures.

The compositions and methods according to the disclosure are especially useful, therefore, for sanitizing, thereby reducing the level of a viable population on the surfaces of buildings where large numbers of individuals may congregate or be confined such as in a hotel or cruise ship, hospital or medical offices, day cares, schools, or military barracks, or where the individuals have access to surfaces where repeated handling or animal or human contact can transmit or have the potential to transmit and cross-contaminate with bacterial and viral organisms.

Advantageously, the present compositions have been found to remain effective even in an organic-rich environment (high organic load). Thus the compositions can be used as a single wash treatment of surfaces such as food preparation surfaces that may contain such materials in addition to pathogenic microbes, but the compositions can also be used as a repeat treatment or a treatment used in conjunction with other cleansers or sanitizers which can further assist in the removal of organic debris.

The term “gelling agent” as used herein refers to such agents as, but not limited to, natural gums, starches, pectins, agar-agar and gelatin, alginic acid, sodium alginate, potassium alginate, ammonium alginate, calcium alginate, agar, carrageenan, locust bean gum, fumed silica, precipitated silica, fine talc, or chalk also viscosity and body while not affecting the target property of a mixture, polyethylene glycol, synthetic polymers such as polyacrylic acid, polyvinyl pyrrolidones, polyethylene glycols, and the like.

The term “cylinder foam test” as used herein refers to a test for measuring both the foamability of compositions and the persistence of the foamed state. In general, the test comprises the steps of placing a test composition into a stoppered, graduated cylinder so that the composition occupies a predetermined height of the cylinder (e.g., about ⅓ to about ½ of the height of the stoppered, graduated cylinder). The stoppered, graduated cylinder is then inverted approximately 10 times to generate a foam. The height of foam is measured immediately after the inverting step as a measure of the foamability of the composition. The foamed composition is then left undisturbed to determine the foam half life (time required for the foam to lose half its height in the graduated cylinder). The cylinder foam test is conducted at room temperature under 1 standard atmosphere pressure (i.e., 100 kPa (about 750.01 mm Hg) or 29.53 in Hg).

### Description

Acid stable, non-enveloped enteric viruses, such as human norovirus and Hepatitis A virus (HAV), are not readily inactivated by treatment with organic acids, surfactants, or detergents. Individually, levulinic acid and SDS provide only minimal (≦2 log CFU/ml) inactivation of viral and bacterial pathogens, but the combination acts synergistically for much greater levels of inactivation.

The need still exists for a composition that comprises generally recognized as safe (GRAS) chemicals, that has efficacy in rapidly killing both enveloped and non-enveloped viruses on all substrates, absent negative impacts to the environment or to the surface to which the composition is applied. It would also be beneficial for such a composition to have long-lasting residual effects, so that the surfaces would remain free of active viruses long after the application of the composition to the surface.

As reported herein, combining a surfactant with an acid synergistically enhances the antimicrobial activity of the respective surfactant and acid. The present disclosure encompasses compositions comprising surfactants, and particularly, but not intended to be limiting in any way, levulinic acid that have a synergistic effect in reducing the viability of a virus population compared to the efficacy of the individual compounds. This synergy allows the formulation of compositions where the active agents (including an acid and a surfactant) are present at concentrations effective to inactivate viruses on surfaces, including human skin, between 10²- and 10⁷-fold.

The levulinic acid/SDS sanitizer compositions of the present disclosure can rapidly (within 1 min) inactivate two infectious surrogates for the pathogenic human NoV, MNV-1, and FCV, using low concentrations (0.5% levulinic acid plus 0.5% SDS) as a liquid solution (1:10, virus to sanitizer). At higher concentrations (5% levulinic acid plus 2% SDS), the sanitizer could inactivate a virus such as MNV-1 on a stainless steel surface when the sanitizer was used as a liquid or foaming treatment (5 min exposure). Furthermore, this concentration of the sanitizer was effective against MNV-1 in the presence of significant amounts of organic material 10% for stainless steel carrier tests and up to 50% in solution tests). It is contemplated, therefore, that the compositions of the disclosure are useful for the destruction of viruses in the presence of organic material present in a clinical matrix (stool or vomit) that would otherwise protect the virus from inactivation by a sanitizer.

Chlorine is an effective disinfectant against most non-enveloped viruses, such as norovirus, when applied in a relatively clean matrix (Cromeans et al., (2010) Appl. Environ. Microbiol. 76: 1028-1033). In the presence of organic material (fecal material, food debris, or DMEM and FBS from cell culture media), greater concentrations of chlorine are required to satisfy chlorine demand. Duizer et al., ((2004) Appl. Environ. Microbiol. 70: 4538-4543) obtained a 3 log PFU/ml inactivation of FCV only after treatment with 3,000 ppm chlorine for 10 min at room temperature when the virus was suspended in cell culture media. The influence of organic material on chlorine disinfection is even more pronounced when viruses are dried onto surfaces. Viable MNV-1 was reduced by >4 log Most Probable Number (MPN)/ml when 1,000 ppm sodium hypochlorite solution was applied to viruses dried on stainless steel surfaces without the presence of food residuals, but minimal reductions in MNV-1 infectivity were observed when food residuals were present on these surfaces (Takahashi et al., (2011) PLoS One 6:e21951).

Although the mechanism of levulinic acid plus SDS for inactivating pathogens is unknown, the reaction is pH dependent. Increasing the sanitizer pH to 4.5 decreased the sanitizer efficacy. Inactivation of FCV by a low-pH sanitizer was anticipated, because FCV is susceptible to inactivation by low pH (Cannon et al., (2006) J. Food Protect. 69: 2761-2765). However, in the same study, MNV-1 was minimally inactivated after a 2-hr exposure at pH 2. The protein degradation potential of anionic detergents are enhanced at low pH, suggesting that the primary target of the sanitizer is the viral capsid.

The combined activity of levulinic acid and SDS was proven to be viricidal for two surrogates for human norovirus infectivity, MNV-1 and FCV, when used in solution. Viricidal activity was also validated for virus dried onto stainless steel surfaces, when concentrations of 5% levulinic acid plus 2% SDS were applied as a liquid or foaming treatment. Foaming sanitizers are of particular interest for large-scale applications, such as for use on cruise ships or in institutional facilities (hospitals, nursing homes, schools, and day-cares). In addition, food service, processing, and harvesting industries are likely to benefit since both active ingredients are generally recognized by the U.S. FDA as safe food additives.

The viricidal compositions disclosed herein are efficacious with little or no damage to the surface to which they may be applied, or even altering the organoleptic properties of a treated food substance. The viricidal compositions and the wipes containing such compositions are suitable for sanitizing any surface suspected of having a viral load thereon or where it is desirable to reduce a viral load. The surfaces to be treated by the compositions of the disclosure include human skin (particularly hands) or surfaces likely to come in contact with human skin, as well as the surfaces of food substances such as poultry, meat or fresh produce, and surfaces that come in contact with food substances such as poultry, meat or fresh produce.

For example, but not intended to be limiting, viable populations of Murine Norovirus on latex gloves were reduced by 4 log PFU/ml after soaking contaminated gloves in 5% levulinic acid plus 2% SDS liquid solution for 5 min at room temperature.

The compositions as described in the present disclosure, while comprising a surfactant and a monoprotic acid as the synergistically cooperating active viricidal agents, may further include such as, but not limited to, L-lysine, peroxacetic acid, N-halamine, D-limonene, hydrogen peroxide, Polysorbate 20, Polysorbate 80, allylisothiocyanate, eugenol, cetylpyridinium chloride, cetylpyridinium bromide, ethanolamine, EDTA, or other compounds that may serve to increase the antiviral activity of the composition.

It is further contemplated to be within the scope of the disclosure for the antiviral compositions herein described to be deposited on or within a flexible base and pliable laminar material that may then be used as a wipe to spread the antiviral composition over a surface desired to be sanitized. The present disclosure, therefore, further encompasses embodiments of a wipe, including a hand wipe or other flexible base material including but not limited to, a fabric, a woven mesh, a pad, a paper towelette, a paper towel, and the like, that may absorb and/or retain thereon a quantity of the liquid antimicrobial composition. The wipe may then be used to apply the antimicrobial solution to a surface that it is desired to sanitize by reducing or eliminating the viability of any microorganisms. Hand wipes can be devised that have both cleansing and sanitizing properties, making them an appealing method of delivery of antimicrobial compositions according to the present disclosure for reducing microbial populations on hand surfaces or other surfaces desired to be substantially freed of potential microbial pathogens. Their efficacy against norovirus, Hepatitis A virus, and Salmonella are of particular interest, given recent foodborne outbreaks associated with contaminated food (Greig et al., (2007) J. Food Prot. 70: 1752-1761).

Suitable flexible base and pliable materials for use in the methods and compositions of the present disclosure include, but are not limited to, a fabric composed partially or entirely of natural fibers including cotton, flax, linen, hemp, and the like, or partially or entirely of artificial materials such as nylon, DACRON™, rayon, polyester, polythene, and the like. The most suitable flexible base materials may be woven or molded as meshes that provide spaces for impregnation of viricidal compositions according to the present disclosure. In one contemplated embodiment, the fibers of the flexible base material may be hollow to absorb an increased amount of the antiviral composition.

It has been found that a particularly useful material for impregnating with the antiviral compositions according to the present disclosure are those materials have a net positive ionic charge thereon. Such materials have an increased capacity to attract negatively charged microorganisms thereby furthering the removal of viral and bacterial particles from a surface and contacting the particles with the antimicrobial composition impregnated in the wipe material.

In one example of the use of an antiviral wipe according to the disclosure, with cellulose wipes soaked in 5% levulinic acid plus 2% SDS solution with an applied 1 Kg per sq. in of pressure, a reduction of 5.53 log Plaque Forming Units/ml (PFU/ml) of Murine Norovirus was achieved when applied to contaminated stainless steel surfaces with two wiping motions over the surface.

Although a wipe impregnated with the antimicrobial composition of the present disclosure provides a convenient means of delivery of the composition to a surface to be sanitized, it is further contemplated that the composition may be contacted with the surface to be treated by a variety of dispensing methods, including by such as, but not limited to, spraying, wiping, dousing, and the like. For example, it was found that a hydraulic spray of a 5% levulinic acid plus 2% SDS liquid solution reduced viable Murine Norovirus populations on stainless steel surfaces by an average of 2.85 log Plaque Forming Units (PFU/ml). Particularly advantageous is applying the composition as a foam that prolongs the application of the antimicrobial to the applied surface and will assist in the physical removal of dislodged viral, bacterial, and organic debris from the treated surface. Accordingly, it is contemplated that the compositions of the disclosure may be formulated to provide a foam having mechanical properties adequate to provide the desired prolonged treatment or debris removal activity. The surfactant component of the compositions herein described can provide the foaming action or additional foaming agents known in the art may be included.

In the antimicrobial compositions of the present disclosure that have antiviral activity, the concentration of total acid present in the composition can be about 0.2 to about 20% by weight per volume in water (2-200 grams/L) and the concentration of total surfactant is about 0.05% to about 5% by weight per volume in water (0.5-50 grams/L). In embodiments thereof, the acid is an acid that has been classified by the U.S. Department of Agriculture as being Generally Regarded As Safe (GRAS) and includes, but is not limited to, levulinic acid, caprylic acid, caproic acid, citric acid, eugenol, adipic acid, tartaric acid, fumaric acid, lactic acid, phosphoric acid, hydrochloric acid, succinic acid, malic acid, and sorbic acid.

The surfactant can be selected from any ionic (cationic or anionic) or non-ionic surfactants. Surfactants for application to the human skin or which might be in contact with foodstuffs consumed by animals or humans preferably should be compatible for human use and not lead to adverse reactions by the recipients. The surfactant component may comprises one or more functionalized organic acids having a hydrocarbon chain length of 2 to 25 carbons, wherein the functionalizing group is selected from hydroxyl, amino carbonyl, sulphonyl, phosphate and thiol groups. Such surfactants are known in the art in the field of food industry and include, for example, sodium dodecyl sulfate (SDS), sodium laureth sulfate (SLS; or sodium lauryl ether sulfate, SLES), cetylpyridinium chloride (CPC), cocamide MEA (MEA), cocamide DEA (DEA), benzalkonium chloride and ethylenediamine tetraacetic acid (H₄EDTA) and salts thereof such as Na₄EDTA and Na₂H₂EDTA. The surfactants used may also include side group substituents attached to the hydrocarbon backbone. Such substituents can be selected from —PO₃, C₁-C₈ hydroxylalkyl and C₅-C₆ aryl hydroxyl groups. The surfactant may also be selected from the group consisting of mono-, di-, tri- and tetra-alkylammonium halides, sulfates and phosphates wherein at least one of the alkyl substituents of the alkylammonium halide comprises at least carbon atoms and more typically 10-25 carbon atoms. In some embodiments, the surfactant can be selected from the group consisting of sodium dodecyl sulfate, sodium laureth sulfate, cetylpyridinium chloride, cetylpyridinium bromide and benzalkonium chloride and the organic acid is levulinic acid.

The compositions disclosed herein may further comprise two or more different acids or two or more different surfactants provided that the total concentration of acid present in the composition is about 0.2% to about 20% by weight per volume in water (2-200 grams/L) and the total concentration of surfactant is about 0.05% to about 5% by weight per volume in water (0.5-50 grams/L). In accordance with one embodiment of the compositions of the disclosure, a viricidal composition can be provided that comprises levulinic acid and a surfactant, where the total concentration of acid in said composition is about 0.5% to about 5.0% (w/v) and the total concentration of surfactant in said composition is about 0.5% to 5% (w/v).

The compositions disclosed herein are capable of reducing resident virus populations in liquids, on solid surfaces, the surfaces of food substance (or surfaces coming in contact with food substances), or on human skin (or on surfaces coming in contact with human skin) by a factor equal to, or greater than, 10², including by a factor of at least 10³, and between a factor of 10³ and a factor of 10⁷, using a combination of an acid and surfactant at concentrations that are ineffective when used separately. Repeat application of the sanitizer compositions of the disclosure can provide an accumulative effect whereby each application can reduce the viral load by 1-3 log so that after three applications the viral load can be reduced by a factor of 7 log or more. The active ingredients of the present compositions (i.e., the acid and surfactant) are individually ineffective in reducing viral cell count by a factor greater than 10², even when the active agents are used separately at 2× or 5× the effective concentration used in the combination.

Accordingly, the antimicrobial (viricidal) compositions of the present disclosure comprise a linear monoprotic organic acid and an ionic long chain (C₈-C₃₀) surfactant. The organic acid is preferably, but not limited to, a linear monoprotic organic acid comprising a carbon backbone of 3 to about 13 carbons. A viricidal composition is provided comprising an acid and a surfactant, wherein the general structure of the acid is CH₃(CH₂)ₘCOOH, with m being an integer selected from 2-12, and the surfactant can be, but is not limited to, sodium dodecyl sulfate (SDS), sodium laureth sulfate (SLS), or sodium lauryl ether sulfate (SLES), cetylpyridinium chloride (CPC) and benzalkonium chloride. In some compositions of the disclosure, the acid has the general structure CH₃(CH₂)ₘCOOH, with m being an integer selected from 2-12 and the surfactant can be sodium dodecyl sulfate (SDS), sodium laureth sulfate (SLS), or sodium lauryl ether sulfate (SLES).

The composition can comprise an acid of the general structure CH₃(CH₂)ₘCOOH, or:

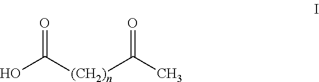

wherein n is an integer selected from 1-10, and the surfactant can be, but is not limited to, a cation of the general structure:

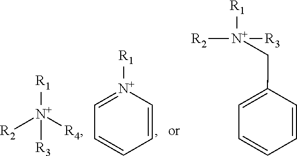

wherein R₁, R₂, R₃, and R₄ are independently selected from the group consisting of C₁-C₂₀ alkyl, and salts thereof. In one embodiment R₁ is C₆-C₂₀ alkyl and R₂, R₃, and R₄ are independently selected from the group consisting of C₁-C₂ alkyl.

Previous studies revealed that combinations of different organic acids can be used as anti-bacterial agents based on their killing effects on E. coli O157:H7 and Campylobacter (Zhao, et al. 2006). Levulinic acid is an organic acid that can be produced cost-effectively and in high yield from renewable feedstocks (Bozell, et al., (2000); Fang & Hanna (2002)). Its safety for humans has been widely tested and FDA has given it GRAS status for direct addition to food as a flavoring agent or adjunct (21 CFR, 172.515). Its application to fresh produce may extend shelf life because levulinic acid can arrest light-induced chloroplast development during greening and can be removed by washing the leaves to restore the developmental process (Jilani et al., (1996) Physiol. Plantarum 96: 139-145).

Sodium dodecyl sulfate (SDS) also has GRAS status (21 CFR, 172.210) at 0.5% wt of gelatin, as a whipping agent in gelatin used in marshmallows and at 0.0125% in liquid and frozen egg whites. It has been widely studied and is used as a surfactant in household products such as toothpastes, shampoos, shaving foams, and bubble baths. The SDS molecule has a tail of 12 carbon atoms attached to a sulfate group, giving the molecule the amphiphilic properties required of a surfactant.

The compositions of the disclosure may optionally further include one or more additional compounds to increase the antimicrobial activity spectrum, to provide a general cleansing activity, and/or to provide a commercially desirable product having a particular odor, color, consistency and the like such as including one or more compounds selected from the group consisting of peroxacetic acid, N-halamine, D-limonene (1-methyl-4-(prop-1-en-2-yl)-cyclohexene), hydrogen peroxide, acidic copper sulfate, an aliphatic alcohol, an aromatic alcohol, a polyquaternium, allylisothiocyanate, eugenol (4-Allyl-2-methoxyphenol), L-lysine, Polysorbate 20 (TWEEN 20™; polyoxyethylene (20) sorbitan monolaurate), Polysorbate 80 (TWEEN 80™; polyoxyethylene (80) sorbitan monooleate), ammonium lauryl sulfate, sodium laureth sulfate, benzalkonium chloride, cetylpyridinium chloride, EDTA, alcohols and polyquaterniums (including for example, polyquaternium-1 [Ethanol, 2,2′,2″-nitrilotris-, polymer with 1,4-dichloro-2-butene and N,N,N′,N′-tetramethyl-2-butene-1,4-diamine]; polyquaternium-2 [Poly[bis(2-chloroethyl)ether-alt-1,3-bis[3-(dimethylamino)propyl]urea]]; polyquaternium-4; polyquaternium-5 [copolymer of acrylamide and quaternized dimethylammoniumethyl methacrylate]; polyquaternium-6 [poly(diallyldimethylammonium chloride)]; polyquaternium-7 [copolymer of acrylamide and diallyldimethylammonium chloride]; polyquaternium-8; polyquaternium-9; polyquaternium-[quaternized hydroxyethylcellulose]; polyquaternium-11 [copolymer of vinylpyrrolidone and quaternized dimethylaminoethyl methacrylate]; polyquaternium-12; polyquaternium-13; polyquaternium-14; polyquaternium-[acrylamide-dimethylaminoethyl methacrylate methyl chloride copolymer]; polyquaternium-16 [copolymer of vinylpyrrolidone and quaternized vinylimidazole]; polyquaterniurn-17; polyquaternium-18; polyquaternium-19.

When the present composition is provided as a foam, the composition has a cellular structure that can be characterized as having several layers of air cells that provide the composition with a foamy appearance. It should be understood that the characterization of a foam refers to the existence of more than simply a few air bubbles and the foam can retain over 20, 30, 40, 50, 60 or 70% of its maximum height in a cylinder foam test 10 minutes after agitation ceases. The foamed antimicrobial composition of the present disclosure can retain at least 20% of its height in a cylinder foam test 5 minutes after agitation is ceased.

Typically, the antimicrobial compositions disclosed herein can be formed as a foam using simple mechanical foaming heads known to those skilled in the art that function by mixing air and the composition to create a foamed composition. However, the use of known chemical foaming mechanisms is also suitable for forming foams in accordance with the present invention. For chemical foaming, the antimicrobial composition can include ingredients that create foam as a result of a chemical interaction, either with other ingredients in the composition, or with substances present in the applicable environment. These components can be provided as a 2-part composition that can be combined when foaming is desired.

Foaming can be accomplished, for example, using a foam application device such as a foaming soap dispenser, tank foamer or an aspirated wall mounted foamer, e.g., employing a foamer nozzle of a trigger sprayer. For example, foaming can be accomplished by placing the composition in a fifteen-gallon foam application pressure vessel, such as a fifteen-gallon capacity stainless steel pressure vessel with mix propeller. The foaming composition can then be dispensed through a foaming trigger sprayer. A wall mounted foamer can use air to expel foam from a tank or line.

The antimicrobial compositions disclosed herein can be optionally administered to a surface as a foam. The foam can be prepared by mixing air with the antimicrobial composition through use of a foam application device. Mechanical foaming heads that can be used according to the invention to provide foam generation include those heads that cause air and the foaming composition to mix and create a foamed composition. That is, the mechanical foaming head causes air and the foaming composition to mix in a mixing chamber and then pass through an opening to create a foam.

Suitable mechanical foaming heads that can be used according to the invention include those available from Airspray International, Inc. (Pompano Beach, Fla.), and from Zeller Plastik, a division of Crown Cork and Seal Co. Suitable mechanical foaming heads that can be used according to the invention are described in, for example, U.S. Pat. No. D-452,822; U.S. Pat. No. D-452,653; U.S. Pat. No. D-456,260; and U.S. Pat. No. 6,053,364. Mechanical foaming heads that can be used according to the invention includes those heads that are actuated or intended to be actuated by application of finger pressure to a trigger that causes the foaming composition and air to mix and create a foam. That is, a person's finger pressure can cause the trigger to depress thereby drawing the foaming composition and air into the head and causing the foaming composition and air to mix and create a foam.

Foam boosting agents can be added to the antimicrobial compositions to enhance either foamability and/or longevity of the formed foam, such as, but not limited to, glycols, glycol ethers, derivatives of glycol ethers, and mixtures thereof. Suitable glycols include those having at least four carbon atoms such as hexylene glycol.

The viricidal compositions of the disclosure can further comprise an anti-foam or suds suppression agent. Incorporation of said agents is particularly desired for applications in which the viricidal compositions will be subjected to agitation in conjunction with the treatment of food substances (e.g., viricidal wash solutions). The viricidal compositions may comprise an anti-foam or suds suppression agent, present at a level of from about 0.0001% to about 15%, or about 0.001% to about 20%, or about 0.005% to about 5.0% by weight of the viricidal composition. Suitable suds suppressing systems for use herein may comprise essentially any known anti-foam compound that exhibits stability at a pH of about 2.0 to about 4.5, including, but not limited to, those selected from the group consisting of silicone anti-foam compounds, silicone emulsions, 2-alkyl and alkanol anti-foam compounds, Anti-foam A, mineral oil emulsions, hydrocarbon oil emulsions, polyalkylene emulsions, and combinations thereof.

Silicone suds suppressors can be the compounded types known for use in antimicrobial compositions, including, for example, polydimethylsiloxanes having trimethylsilyl or alternate endblocking units. Such compounds may be compounded with silica and/or with surface-active non-silicon components, as illustrated by a suds suppressor comprising 12% silicone/silica, 18% stearyl alcohol and 70% starch.

The viricidal composition according to the disclosure can comprise about 0.05 to about 5% sodium dodecyl sulfate by weight per volume in water, about 0.2 to about 20% levulinic acid by weight per volume in water and, optionally: 1) about 0.05% to about 70%, or about 0.05% to about 62%, or about 0.05% to about 20% or about 0.05% to about 1%, of an alcohol solvent. In one embodiment the alcohol solvent is about 50% to about 80% or about 75% to about 85%. Suitable alcohol solvents include, but are not limited to, ethanol, propanol, isopropanol, butanol, propylene glycol, diethylene glycol, dipropylene glycol and mixtures thereof; 2) about 0.05% to about 20% or about 0.05% to about 5%, or about 0.05% to about 1% of a cationic agent selected from the group consisting of benzalkonium chloride, benzethonium chloride, triclocarban, tricolsan, chlorhexidine and mixtures thereof; 3) about 0.05% to about 5%, or about 0.05% to about 2%, or about 0.05% to about 1% of a heavy metal salt selected from the group consisting of silver, zinc, copper and mixtures thereof.

The present disclosure further provides a composition substantially free of a solvent and comprising: a surfactant; and a monoprotic organic acid comprising a carbon backbone of 3 to 13 carbons, where the pharmaceutically acceptable surfactant and the monoprotic organic acid are in a weight ratio of between about 1:400 to about 25:1 that when added to an appropriate solvent will provide an antimicrobial (antiviral) composition according to the present disclosure.

The viricidal compositions disclosed herein can be used to reduce the population of an undesirable virus on a surface. Successful reduction of a population of a virus can be achieved when the infectivity of a virus population is reduced by at least 2 log. The viricidal compositions can be used to inactivate a wide range of viruses including, but not limited to, gastroenteritis viruses and their surrogates, caliciviruses such as Murine norovirus, Feline Calicivirus, Human Norovirus and Sapovirus, Human Rotavirus, Sapovirus, Astrovirus, Bocavirus; Hepatitis viruses, such as Hepatitis A and Hepatitis E virus; and respiratory viruses, such as Rhinovirus, Corona virus, Influenza virus, and Adenovirus serotypes.

The viricidal compositions can be formulated in various carriers for administration to inactivate viruses on a surface. For example the compositions can be formulated as a hand sanitizer (either as a water based or water free formulation) using standard techniques known to those skilled in the art. Similarly the composition can be added to fibrous materials to formulate hand sanitizing wipes or towelettes for sanitizing hard surfaces. Such sanitizing wipes may further include such components as alumina nanofibers, charged glass and the like, and which may aid in attracting charged microorganisms from the contaminated surface. These additional compounds can be interwoven in such as NANOCERAM™ filters.

In addition, the viricidal compositions can be formulated as a packaging insert for fresh produce or meat products consisting of a cellulose-based material soaked in viricidal composition, wherein any virus present will be inactivated upon food contact with the insert. The viricidal compositions can be encapsulated (using standard techniques) to provide delayed or prolonged release of the active components.

In one embodiment a packaging insert for fresh produce or meat products is provided which consists of a cellulose based material soaked in a slow-release viricide composition for virus inactivation within the package (not necessarily coming into contact with the insert). Alternatively, the viricidal composition can be provided as a foodstuff wash solution, optionally containing an antifoaming agent. In an additional embodiment the composition is provided as a foaming decontamination spray for use on hard surfaces (especially in cruise-ships, daycares, hospitals, and the like).

Accordingly, it was determined that low concentrations of levulinic acid plus sodium dodecyl sulfate solution were rapidly effective against the Human Norovirus surrogates, Murine Norovirus (MNV), and FCV and the agent of the common cold, Human Rhinovirus. As little as 0.5% levulinic acid plus 0.5% sodium dodecyl sulfate provided a >3 log reduction in PFU/ml of virus infectivity in less than 1 min. MNV was rapidly inactivated on stainless steel surfaces using a 5% levulinic acid plus 2% sodium dodecyl sulfate solution and a 5% levulinic acid plus 2% sodium dodecyl sulfate foam, resulting in >3 log reductions of MNV within 5 min of treatment, regardless of the presence of up to 10% organic material. The levulinic acid plus sodium dodecyl sulfate solution disclosed herein, therefore, demonstrates rapid efficacy against the Human Norovirus surrogates, MNV, and FCV in solution and when used on the surfaces of stainless steel and in the context of organic debris. The liquid sanitizer was also effective against Human Rhinovirus and, therefore, provides an alternative to alcohol-based sanitizers which have limited efficacy against non-enveloped viruses. Combinations of 5% levulinic acid plus 2% sodium dodecyl sulfate were more effective than lower concentrations of levulinic acid plus sodium dodecyl sulfate (2% levulinic acid plus 1% sodium dodecyl sulfate and 5% levulinic acid plus 2% sodium dodecyl sulfate) and water.

Dry wipes, regardless of charge or number of wipes were ineffective, resulting in <1 log for removal of norovirus, Hepatitis A virus, and Salmonella enterica. In contrast, wet wipes, particularly those carrying a cationic charge on the base material and those impregnated with levulinic acid plus sodium dodecyl sulfate sanitizer effectively reduced populations of MNV on stainless steel surfaces by >1 log. Multiple (5) wiping passages (motions) in most of the tested scenarios was more effective than a single wipe and the 5% levulinic acid plus 2% sodium dodecyl sulfate solution effectively reduced MNV on stainless steel surfaces by >2 log after 5 wipes.

The present disclosure encompasses the uses of antimicrobial compositions effective in reducing the viability of a wide spectrum of microbial types including, but not limited to, viruses, bacteria (both non-sporing and sporing), yeasts, fungi, and the like. In particular, the compositions off the disclosure have been formulated to be effective in reducing the viability of viruses that are frequently the causative agent of human illnesses. It is also within the scope of the disclosure for the antimicrobial compositions to be combined with additives such as, but not limited to, soaps, foaming agents, gelling agents, colorants, fragrances, and the like that can facilitate the dispensation of the antimicrobials to a surface, including hard surfaces, skin surfaces, food or liquids. Particularly useful is the inclusion of the antimicrobials of the present disclosure in hand washes and sanitizing wipes that have application in reducing the transference of such as viruses or pathogenic organisms by direct contact of a human or animal with a source of the organisms.

It has been found that the combination of a surfactant such as sodium dodecyl sulfate (SDS) with levulinic acid results in synergistic antimicrobial effectiveness not seen with the individual compounds. Accordingly, this surprising synergy allows the formulation of compositions having a pH value less than about 7.0 wherein the active agents are present at concentrations effective in reducing microbial counts by a factor between 10² and 10⁷. The compositions of the disclosure are benign and can readily be tolerated by skin, mucosal surfaces, and do not alter organoleptic properties of treated food substances. The active agents are FDA-approved as food additives. Thus the compositions of the disclosure have wide, if not universal, applicability, providing environmentally acceptable benefits with significant cost benefits. They avoid the use of such universal antimicrobials based on the inclusion or generation of chlorine, that while effective as an anti-viral, anti-bacterial agent has the capacity to damage treated materials, or irritate skin or tissues of a human or animal.

Accordingly, an antimicrobial composition is provided herein comprising a pharmaceutically acceptable acid and a pharmaceutically acceptable surfactant. Surprisingly, the compositions disclosed herein are capable of reducing a microbial population of a liquid or a surface in contact with or likely to be in contact with a microorganism, by a factor greater than 10², including by a factor of 10³ to a factor of 10⁸, using a combination of an acid and surfactant at concentrations that are ineffective when used separatedly. The active ingredients of the present compositions (i.e., the pharrmaceutically acceptable acid and surfactant) are individually ineffective in reducing microbial cell count by a factor greater than 10², even when the active agents are used at 2× or 5× the effective concentrations of the combination.

Combinations of different organic acids can be used as anti-bacterial agents, as measured on their killing effects on the sample species E. coli O157:H7 and Campylobacter (Zhao, et al. 2006). Levulinic acid is an organic acid that can be produced cost effectively and in high yield from renewable feedstocks (Bozell, et al. 2000, Fang & Hanna, 2002). Its safety for humans has been widely tested and FDA has given it GRAS status for direct addition to food as a flavoring agent or adjunct (21 CFR, 172.515). As disclosed herein, the antimicrobial effect of 1% by weight levulinic acid alone will not suffice to kill more than about 1 log CFU Salmonella/ml within 30 minutes; its bactericidal effect was increased only to about 3.4 log CFU/ml within 30 minutes when the levulinic acid concentration was increased to 3% by weight, as shown in Tables 6-8.

Sodium dodecyl sulfate (SDS) also has GRAS status (21 CFR, 172.210) at 0.5% wt of gelatin used as a whipping agent or in marshmallows, and at 0.0125% in liquid and frozen egg whites. It is used as a surfactant in household products such as toothpastes, shampoos, shaving foams, and bubble baths. The SDS molecule has a tail of 12 carbon atoms attached to a sulfate group, giving the molecule the amphiphilic properties required of a surfactant. However, as disclosed herein, SDS by itself has very little antimicrobial effect, so that its use as an antimicrobial in current compositions and products likely resides in its ability to mechanicaly dislodge contaminants from a microbially colonized surface.

In contrast, the substantial microbicidal effect of a combination of levulinic acid and SDS on E. coli O157:H7 and Salmonella was validated even in an extreme organic-rich environment (water containing fecal matter or feathers), as shown in Tables 9-12.

The embodiments of the methods of the present disclsoure, therefore, provide for contacting a surface such as, but not limited to, a skin surface or a hard surface including building fittings, furniture, and the like, with an antimicrobial composition having a pH value of less than 7.0 and comprising pharmaceutically acceptable surfactant and a pharmaceutically acceptable organic acid. It is further contemplated that in embodiments of the compositions of the disclosure, the concentration of the pharmaceutically acceptable acid in the antimicrobial composition is within the range of about 0.03% to about 20%, about 0.03% to about 10%, about 0.03% to about 5%, about 0.03% to about 3%, or about 0.05% to about 2%, or about 0.05% to about 1%, or about 0.1% to about 3%, or about 0.3% to about 3%, or about 0.3% to about 2%, or about 0.5% to about 3%, or about 0.5% to about 2%, or about 0.5% to about 1%, weight per volume in water. In one embodiment the concentration of the pharmaceutically acceptable surfactant in the anitmicrobial composition is within the range of about 0.005% to about 1%, or about 0.01% to about 1%, or about 0.05% to about 1%, or about 0.1% to about 1%, or about 0.05% to about 2%, or about 0.5% to about 2% by weight per volume in water.

A method for the rapid killing of microbial strains, including viruses, bacteria, yeasts, and molds found on physical objects associated with food preparation, cooking, and serving is also provided. The method comprises contacting an object in a food processing environment, including, but not limited to, the equipment required for bottling a beverage, or the containers such as bottles, with the antimicrobial compositions of the disclosure.

Processing equipment is commercially available for washing beverage containers, and applicants have found that the levulinic compositions of the present invention (eg. compositions having a concentration up to 3% levulinic acid) are not corrosive to such equipment. In particular, applicants have found that when using a large stainless steel seed washing unit, not only was the levulinic acid/SDS treatment as effective in killing E. coli O157:H7 as the current industrial standard of 20,000 ppm calcium hypochlorite, but it was not corrosive to the equipment and even removed rust on chains within the unit. Thus the levulinic acid composition served to clean the unit like a detergent without the undesirable corrosive effect on equipment that is associated with many sanitizers such as chlorine. Accordingly, one embodiment of the present invention is also directed to a method of decontaminating equipment and hard surfaces by contacting such equipment and hard surfaces with the levulinic compositions of the present disclsoure.

The methods of the disclosure, therefore, comprise contacting the liquid or surface with a composition comprising levulinic acid and SDS, wherein the composition comprises a maximum concentration of 3% by weight levulinic acid and 2% by weight SDS. In one aspect of the disclosure, the antimicrobial compositions may be included with a wipe material, thereby providing a convenient means of dispensing the composition to a skin, e.g. a hand surface, or other, inert, surface that it is desired to decontaminate. In the alternative, the antimicrobial compositions may be formulated as a liquid, a gel, a foam, and the like for dispensing the composition over large surface areas, or to a localized area.

One aspect of the disclosure encompasses embodiments of an antimicrobial composition comprising: a monoprotic organic acid comprising a carbon backbone of 3 to 13 carbons having the general structure of:

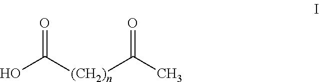

where n is an integer selected from 1 to 10, and where the concentration of the acid in said composition can be about 0.2% to about 20% by weight per volume of solvent; a surfactant, having a concentration about 0.05% to about 5% by weight per volume of solvent; and an aqueous solvent, where the antimicrobial composition is formulated to be effective in reducing the viability of a viral population, a bacterial population, a fungal population, or of any combination thereof.

In embodiments of this aspect of the disclosure, the antimicrobial composition can be formulated to be effective in reducing the viability of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the surfactant can be an anionic surfactant selected from the group consisting of: sodium dodecyl sulfate, sodium laureth sulfate, cetylpyridinium chloride, cetylpyridinium bromide, and benzalkonium chloride.

In embodiments of this aspect of the disclosure, the antimicrobial composition can further comprise a gelling agent, a foaming agent, a soap, a colorant, a fragrance, or any combination thereof.

In embodiments of this aspect of the disclosure, the antimicrobial composition can be formulated as a liquid; a foam having a cylinder foam test half-life of at least ten minutes, or a mix precursor thereof; a gel; or a solid or semi-solid soap.

In embodiments of this aspect of the disclosure, the solvent can be water or an alcohol:water, where the alcohol can be selected from the group consisting of ethanol, propanol, isopropanol, butanol, propylene glycol, diethylene glycol, dipropylene glycol, or any mixture thereof.

In embodiments of this aspect of the disclosure, the antimicrobial composition can further comprise a cationic agent selected from the group consisting of: benzalkonium chloride, benzethonium chloride, triclocarban, tricolsan, chlorhexidine, and any combination thereof.

In the embodiments of this aspect of the disclosure, the composition can be selected from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; and about 5% levulinic acid and about 2% sodium dodecyl sulfate by weight per volume solvent.

In embodiments of this aspect of the disclosure, the antimicrobial composition can be deposited on or within a flexible support material.

In these embodiments of this aspect of the disclosure, the flexible support material can be a cloth, a fabric, a paper, a natural fiber mesh, a synthetic fiber mesh, a combination natural and synthetic fiber mesh, a brush-like surface, or a porous fabric.

In embodiments of this aspect of the disclosure, the antimicrobial composition can be substantially free of a solvent, wherein the pharmaceutically acceptable surfactant and the monoprotic organic acid are in a weight ratio of between about 1:200 to about 16.6:1.

Another aspect of the disclosure encompasses embodiments of sanitizing wipe comprising a flexible support material and an antimicrobial composition absorbed thereon, where the antimicrobial composition can comprise levulinic acid, sodium dodecyl sulfate, and a solvent, where the total concentration of the levulinic acid is about 0.2% to about 20% by weight per volume of solvent and the total concentration of the sodium dodecyl sulfate is about 0.05% to about 5% by weight per volume of solvent, and where the antimicrobial composition can be formulated to be effective in reducing the viability of a microbial population.

In embodiments of this aspect of the disclosure, the antimicrobial composition can be formulated to be effective in reducing the viability of a viral population, a bacterial population, a fungal population, or of any combination thereof.

In some embodiments of this aspect of the disclosure, the antimicrobial composition can formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the flexible support material can have a surface positive charge thereon.

In embodiments of this aspect of the disclosure, the solvent can be water or an alcohol:water mix, where the alcohol can be selected from the group consisting of ethanol, propanol, isopropanol, butanol, propylene glycol, diethylene glycol, dipropylene glycol, or any mixture thereof.

In embodiments of this aspect of the disclosure, the composition can further comprise a cationic agent selected from the group consisting of: benzalkonium chloride, benzethonium chloride, triclocarban, tricolsan, chlorhexidine, and any combination thereof.

In embodiments of this aspect of the disclosure, the can be selected from the group consisting of about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; and about 5% levulinic acid by weight per volume solvent and about 2% sodium dodecyl sulfate by weight per volume solvent.

In embodiments of this aspect of the disclosure, the composition can further comprise a gelling agent, a foaming agent, a soap, a colorant, a fragrance, or any combination thereof.

In embodiments of this aspect of the disclosure, the flexible support material can be a cloth, a fabric, a paper, a natural fiber mesh, a synthetic fiber mesh, a combination natural and synthetic fiber mesh, a brush-like surface, or a porous fabric.

Yet another aspect of the disclosure encompasses embodiments of a method of reducing the viability of a microbial population, the method comprising contacting a microbial population with an antimicrobial composition comprising about 0.2% to about 20% by weight of levulinic acid per volume of solvent, about 0.05% to about 5% by weight of sodium dodecyl sulfate per volume of solvent, and an aqueous solvent, whereby the viability of the population of viruses is reduced.

In embodiments of this aspect of the disclosure, the microbial population can be on a non-liquid surface. In other embodiments of this aspect of the disclosure, the microbial population can be on a skin surface.

In embodiments of this aspect of the disclosure, the antimicrobial composition can be formulated to be effective in reducing the viability of a viral population, a bacterial population, a fungal population, or of any combination thereof.

In embodiments of this aspect of the disclosure, the antimicrobial composition can be formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

In embodiments of this aspect of the disclosure, the composition can be selected from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume; and about 5% levulinic acid by weight per volume solvent and about 2% sodium dodecyl sulfate by weight per volume solvent.

In embodiments of this aspect of the disclosure, the composition can be disposed on a flexible support material. In some embodiments of this aspect of the disclosure, the flexible support material can include a positive ionic charge thereon.

In embodiments of this aspect of the disclosure, the antimicrobial composition can be applied to a viral population is formulated as a liquid wash, a spray, a foam, a paste, a cream, a gel, or a wipe.

In embodiments of this aspect of the disclosure, the antimicrobial composition can be formulated to be effective in reducing the viability of a microbial population on a skin surface, where the microbial population is a viral population, a bacterial population, a fungal population, or any combination thereof, and wherein the antimicrobial composition is applied to the microbial population as a liquid wash, a spray, a foam, a paste, a cream, a gel, or a wipe.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 21° C. and 1 atmosphere.

It should be noted that ratios, concentrations, amounts, and other numerical data may be expressed herein in a range format. It is to be understood that such a range format is used for convenience and brevity, and thus, should be interpreted in a flexible base manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 5%, and 4%) and the sub-ranges (e.g., 0.5%, 1.1%, 2.2%, 3.5%, and 4.4%) within the indicated range. The term “about” can include ±1%, ±2%, ±5%, ±4%, ±5%, ±6%, ±7%, ±8%, ±9%, or ±20%, or more of the numerical value(s) being modified.

### EXAMPLES

### Example 1

Virus cultivation and plaque assay: RAW 264.7 cells (ATCC# TIB-71), Crandell Reese Feline Kidney (CRFK) cells (ATCC# CCL-94) were maintained in either (a) complete Dulbecco's Modified Eagles Medium (DMEM) (Fisher Scientific #SH30081 LS) containing 20% low endotoxin fetal bovine serum (FBS) (HyClone, Logan, Utah) for RAW 264.7 cells, or (b) 20% FBS (Atlanta Biological, GA) for CRFK cells. HeLa-Ohio cells were maintained in complete Modified Eagles Medium (MEM) (Fisher scientific #SH30024LS) containing 20% fetal bovine serum (FBS). RAW 264-7 DMEM was supplemented with penicillin (100 μml), streptomycin (100 μg/ml) with 100 mM HEPES, and 1 mM sodium pyruvate, CRFK DMEM was supplemented with penicillin (100 U/ml), streptomycin (100 μg/ml), 1% L-glutamine and 1% non-essential amino acids. Complete MEM for HeLa-Ohio cells was supplemented with penicillin (100 U/ml), streptomycin (100 μg/ml), 1 mM sodium pyruvate and 1% non-essential amino acids. Murine norovirus (MNV), Feline Calicivirus (FCV) (ATCC # VR-2057), and Human Rhinovirus-16 were cultured by infecting 80-90% confluent monolayers of RAW 264.7, CRFK, HeLa-Ohio cells, respectively in complete maintenance medium.

Virus was harvested after complete CPE (cytopathic effect) was apparent (typically after 48 h) by three cycles of freeze-thawing. Cellular debris was removed by centrifugation for 10 min at 3,600×g and the supernatant was filtered through a 0.2 μm membrane filter (Millipore, Billerica, Mass.) before storing as 1-4 ml aliquots at −70° C. until use. For experiments requiring a high titer of virus stock, additional concentration by ultracentrifugation (100,000×g for 1 hr at 4° C.) was performed. Pelleted virus was suspended in PBS (pH 7.2) containing 0%, 5%, or 10% FBS (Atlanta Biological) overnight prior to use or storage at −70° C.

### Example 2

Quantification of virus infectivity: To determine the infectious titer of MNV and FCV, standard plaque assay techniques were employed (Cannon et al., (2006) J. Food Prot. 69: 2761-2765, incorporated herein by reference in its entirety). Briefly, cells were dispensed in 60 mm diameter cell culture plates at a density of 2×10⁶ cells per plate and grown to 80-90% confluence in complete DMEM. Immediately preceding infection, the cell culture media was replaced with 0.5 ml of complete MEM without phenol red (Cellgro, Mediatech, Inc, Manassas, Va.), supplemented with either (a) 5% low endotoxin FBS, penicillin (100 U/ml), and streptomycin (100 μg/ml) with 100 mM HEPES, and 10 mM sodium pyruvate for MNV or (b) 4% FBS, 1% L-glutamine and 1% non-essential amino acids for FCV.

Ten-fold serial dilutions of virus were prepared in phosphate buffered saline (PBS) pH 7.5 and cell monolayers were infected in duplicate with 0.1 ml of each virus dilution, and 0.5 ml of complete MEM (see below) for 1 hr at 37° C. and 5% CO₂ with gentle rocking every 15 min. Subsequently, the cells were overlaid with complete MEM (without phenol red) (Cellgro) supplemented as described above but also containing 0.5% agarose (SeaKem GTG, Lonza, Rockland, Me.). Viruses were incubated for 48 hr at 37° C. and 5% CO₂. Plaques were subsequently counted 5-8 hr after a second agarose overlay (0.5% agarose diluted in deionized water and including 1% neutral red solution (Sigma-Aldrich, St. Louis, Mo.)) was added. Plates with 5 to 50 plaques were used to determine the virus titer in plaque forming units (PFU/ml).

To determine the infectious titer of HRV-16, a Most Probable Number (MPN) technique was employed. HeLa-Ohio cells were grown to 80-90% confluence in 96-well tissue culture dishes in complete MEM. Virus was serially diluted (10×) in PBS and inoculated onto eight replicate wells (50 μl each) per sample. Cells were incubated for 1 hr at 33° C. with 5% CO₂ with gentle rocking every 15 min. After removal of inoculums, cells were supplemented with complete MEM and incubated for an additional 48 hrs. Replicate wells were visually scored (positive for cytopathic effect (CPE) or negative) for virus infection and MPNs were determined using the Build 23 MPN Calculator. Virus log reductions for each treatment were determined by comparison to a positive control (serial dilution of virus stock).

### Example 3

Determination of MNV, FCV, and HRV-16 inactivation by levulinic acid plus sodium dodecyl sulfate solution: Working concentrations of levulinic acid plus sodium dodecyl sulfate (SDS) (both from Sigma-Aldrich, St. Louis, Mo.) were prepared from 20% or 98% stock solutions by dilution in sterile, ultra-purified, de-ionized water on the day of each experimental trial. Partially purified virus cell culture lysate or concentrated virus cell culture lysate (approximately 3×10⁶ PFU/ml stock; 0.1 ml) was added to each concentration of levulinic acid plus sodium dodecyl sulfate solution (0.9 ml) and mixed on a shaking platform (200 rpm) at 21° C. At each time interval (0 secs, 20 secs, 40 secs, 1 min or 5 min), 0.1 ml of the solution was removed and immediately diluted 1:10 (for all concentrations less than or equal to 2% levulinic acid plus 1% sodium dodecyl sulfate) or 1:1000 (for concentrations greater than 2% levulinic acid plus 1% sodium dodecyl sulfate) in complete DMEM containing 20% FBS for neutralization. Samples were then 10-fold serially diluted in PBS or infection medium (1× MEM containing 5% FBS) and inoculated onto cell culture monolayers. Virus inoculated in water and processed identically to the experimental trials served as a control for determining the virus reduction by the levulinic plus sodium dodecyl sulfate solution. Positive and negative control experiments were performed in duplicate and parallel with each experimental trial. Neutralization controls for which virus was added to sanitizer subsequent to sanitizer neutralization were also included for each experiment with each virus.

### Example 4

Determination of cytotoxic effects of levulinic acid plus sodium dodecyl sulfate on RAW 264.7 cells: Working concentrations of levulinic acid plus sodium dodecyl sulfate (as listed in Table 1) were prepared as described above, mixed on a shaking platform (200 rpm) for 2 min at 21° C., and diluted 1:10 in 0.1M PBS to for neutralization. Duplicate cell culture monolayers were inoculated with 100 μl of ten-fold serial dilutions (in 0.01M PBS) of each levulinic acid plus sodium dodecyl sulfate solution and 100 μl of MNV cell culture lysate (diluted to approximately 70 PFU/ml). To quantify cell cytotoxicity caused by the levulinic acid plus sodium dodecyl sulfate solution, plaques numbers on duplicate cell culture plates were averaged for each treatment group and compared to control plates containing the same quantity of virus, but without containing the levulinic acid plus sodium dodecyl sulfate solution.

### Example 5

Determination of MNV inactivation by levulinic acid plus sodium dodecyl sulfate on stainless steel and produce surfaces with liquid or foaming treatments: Sterile stainless steel coupons (4 cm×2.5 cm with a No. 4 finish) or red grapes were inoculated with 100 μl or 10 μl of MNV partially purified cell culture lysate (approximately 7×10⁶ PFU/ml stock) or concentrated cell culture lysate (approximately 6×10⁷ PFU/ml stock) and allowed to dry in a BSC-2 for 30-40 min at 21° C., or until visibly dry. Levulinic acid plus sodium dodecyl sulfate solutions prepared at 2% levulinic acid plus 1% sodium dodecyl sulfate, 5% levulinic acid plus 2% sodium dodecyl sulfate, 5% levulinic acid plus 1% sodium dodecyl sulfate or 5% levulinic acid plus 2% sodium dodecyl sulfate concentrations were used as a liquid solution (50 ml) or aerated by a hand pumping device (foaming soap container) to create a foaming solution (approximately 25 ml) that was applied to completely cover the virus inoculated stainless steel coupons. At each time interval (1 or 5 min), the coupon or grape was transferred to a 50 ml conical tube containing 10 ml of complete DMEM containing 20% FBS and gently rocked for 5 seconds to neutralize the sanitizer. The stainless steel coupon or grape was then transferred to a 50 ml conical tube containing 10 ml of 0.1M PBS with 1M NaCl and vigorously vortexed to elute viruses from the stainless steel coupon. A non-inoculated stainless steel coupon and uninoculated grape was also processed and served as a negative control. Neutralization controls, where virus was added to 50 ml conical tubes containing the elution buffer after sanitizer neutralization, were also included. Recovery of virus dried on the surface of untreated controls processed identically to the experimental trials were included as positive controls and served as a comparison for determining virus log reduction after each treatment. Sanitizer efficacy was compared to virus removal by treatment with sterile deionized water in a procedure identical to that of the sanitizer treatment experiments.

### Example 6

Statistical analysis: Each experimental treatment was performed in triplicate and a positive control experiment (MNV in water) was included with each trial. Log concentrations of MNV were calculated based on average plaque counts of duplicate cell culture plates and the dilution factor. Average MNV log reductions were calculated based on subtraction of experimental trial counts from control trial counts. Because all levulinic acid plus sodium dodecyl sulfate combinations greater than 0.5% w/v produced cytotoxicity in neutralized, undiluted samples, the assay limit of detection was reduced by 1.0 log units for the experimental results in Table 1. For statistical analysis, log reductions of 3.0 PFU/ml or greater were assigned a value of 3.0 PFU/ml. A two-tailed student's T-test (Microsoft Excel) was used to determine significant differences (P 0.05) between the experimental treatment and control groups.

### Example 7

Norovirus surrogate inactivation by levulinic acid or SDS: When prepared individually in solution, neither levulinic acid (0.5%-3%) nor SDS (0.05%-2%) was effective in inactivating the norovirus surrogates, MNV-1 or FCV (as shown in Table 1). Average log PFU/ml reductions in infectious virus titers were ≦0.51 log PFU/ml after 1 min of exposure to each treatment. There were no significant differences between the log PFU/ml reductions of MNV-1 and FCV at any concentration of levulinic acid or SDS tested (p≧0.05).

TABLE 1
Log reduction in PFU/ml of viable MNV-1 and FCV
after treatment with different concentrations
of SDS or levulinic acid at 21° C. for 1 min.

Log Reduction PFU/ml (standard deviation)^{a}

| Treatment type | MNV-1 | FCV |
|---|---|---|
| 0.05% SDS | −0.13 (±0.26) | −0.12 (±0.23) |
| 0.5% SDS | 0.13 (±0.20) | −0.09 (±0.27) |
| 1% SDS | 0.03 (±0.12) | −0.05 (±0.18) |
| 2% SDS | −0.23 (±0.59) | −0.05 (±0.22) |
| 0.5% Levulinic | 0.09 (±0.38) | 0.09 (±0.16) |
| acid | | |
| 1% Levulinic acid | −0.04 (±0.23) | 0.32 (±0.19) |
| 2% Levulinic acid | −0.06 (±0.17) | 0.51 (±0.34) |
| 3% Levulinic acid | −0.09 (±0.10) | 0.43 (±0.23) |

^{a}Standard deviations are indicated in parenthesis.

### Example 8

All concentrations of levulinic acid plus sodium dodecyl sulfate solution began to inactivate MNV almost immediately, resulting in at least 1.3 log reduction in PFU/ml of viable MNV upon contact with the solution (time=0), as shown in Tables 2-4.

TABLE 2
Inactivation of MNV-1 after treatment with
levulinic acid (LVA) plus SDS solution.

| | Log Count PFU/ml (standard deviation) | | |
|---|---|---|---|
| | Time of Treatment | | |
| Treatment Type | Time 0 sec | Time 20 sec | Time 40 sec |
| Sterile Water Control | 6.77 (±0.13) | 6.91 (±0.25) | 6.91 (±0.30) |
| 0.5% LVA + 0.5% SDS | 5.51 (±0.31) | 4.06 (±1.20) | <2.70^{c} (±0.00) |
| 0.5% LVA + 1% SDS | 5.31 (±0.29) | 4.22 (±0.07) | <2.70 (±0.00) |
| 1% LVA + 1% SDS | 4.60 (±0.79) | 3.47 (±0.68) | <2.70 (±0.00) |
| 1% LVA + 2% SDS | 4.72 (±0.55) | 3.47 (±0.68) | <2.70 (±0.00) |
| 2% LVA + 1% SDS | 4.26 (±1.83) | 3.03 (±0.65) | <2.70 (±0.00) |

TABLE 3
Log reduction in PFU/ml of Murine Norovirus after treatment with
levulinic acid plus SDS solution at 21° C. (n = 3).
MNV log reduction after treatment with levulinic acid
plus sodium dodecyl sulfate (SDS) wash solution

| | Time 0 sec | Time 20 sec | Time 40 sec |
|---|---|---|---|
| Water | 0 (±0) | 0 (±0.2) | 0 (±0.2) |
| 1% levulinic acid + | 2.2 (±0.7)* | >3.0 (±0)* | >3.0 (±0)* |
| 1% SDS | | | |
| 1% levulinic acid + | 1.5 (±1.1)* | 2.6 (±0.7) | >3.0 (±0)* |
| 0.5% SDS | | | |
| 0.5% levulinic acid + | 1.3 (±0.3)* | 2.6 (±0.4)* | >3.0 (±0)* |
| 0.5% SDS | | | |
| 1% levulinic acid + | 2.1 (±0.5)* | >3.0 (±0)* | >3.0 (±0)* |
| 2% SDS | | | |
| 0.5% levulinic acid + | 1.5 (±0.2)* | 2.8 (±0.3)* | >3.0 (±0)* |
| 1% SDS | | | |
| 2% levulinic acid + | 2.0 (±1.2)* | >3.0 (±0)* | >3.0 (±0)* |
| 1% SDS | | | |

*Indicates significant difference from water control (P < 0.05)

TABLE 4
Inactivation of MNV-1 and FCV after treatment with levulinic
acid plus SDS solution at 21° C. for 1 min.

| | Log Count PFU/ml (standard deviation)^{a} | |
|---|---|---|
| Treatment Type | MNV-1 | FCV |
| Sterile Water Control | 6.91 (±0.30) | 5.74 (±1.28) |
| 0.5% levulinic acid + | 5.89 (±0.19) | <2.70^{b} (±0.00) |
| 0.05% SDS | | |
| 0.5% levulinic acid + | <2.70^{b} (±0.00) | <2.70 (±0.00) |
| 0.5% SDS | | |
| 2% levulinic acid + 1% SDS | <2.70 (±0.00) | <2.70 (±0.00) |

^{a}Standard deviations are indicated in parenthesis.
^{b}Counts below the lower detection limit of the assay are indicated as <2.70 log PFU/ml.

Within 20 sec, a >3.0 log reduction in MNV viability was observed after treatment with combinations of levulinic acid plus sodium dodecyl sulfate that were at least 1% for each component. Extending the treatment time to 40 sec resulted in a >3.0 log reduction in MNV viability for all concentrations of the levulinic acid plus sodium dodecyl sulfate solution. With the assay limit of detection at 3.0 log, no viable virus could be detected by plaque assay after each 40 sec treatment, whereas controls (MNV inoculated in water) remained infectious throughout the duration of the experiment with no reduction in virus infectivity (n=5; mean log reduction=0, standard deviation=0.2).

All concentrations of the levulinic acid plus sodium dodecyl sulfate solution (greater than 0.5% w/v) produced cytotoxicity after neutralization unless diluted 10 fold. Elimination of cytotoxicity by the solution was demonstrated by inoculating duplicate cell culture monolayers with 10 fold serial dilutions of the neutralized levulinic acid plus sodium dodecyl sulfate solution and a known concentration of MNV (approximately 70 PFU/ml). Control experiments performed in parallel included only the known concentration of MNV. When compared to controls, no reduction in plaque numbers was observed for any of the treatments that were diluted at least fold (n=3, P values≧0.1).

### Example 9

All concentrations of levulinic acid plus sodium dodecyl sulfate solution greater than or equal to 0.5% levulinic acid plus 0.5% sodium dodecyl sulfate inactivated MNV, FCV and HRV-16 by 3 log or greater within 1 min With lower concentrations of SDS (0.5% levulinic acid plus 0.05% sodium dodecyl sulfate), FCV was inactivated by >3.6 log, MNV was reduced by only 1.04 log, as shown in Table 5.

TABLE 5
Log reduction in PFU/ml of MNV, FCV and HRV-16
after treatment with levulinic acid plus SDS solution
at 21° C. for 1 min (n = 3).

| | MNV | FCV | HRV-16 |
|---|---|---|---|
| 0.5% levulinic acid + | 1.04 (±0.17) | >3.6 (±0.17) | ND |
| 0.05% SDS | | | |
| 0.5% levulinic acid + | >4.00 (±0.34) | >3.6 (±0.17) | >4.38 (±0.0) |
| 0.5% SDS | | | |
| 2% levulinic acid + | >3.08 (±0.13) | ND | >4.94 (±0.0) |
| 1% SDS | | | |

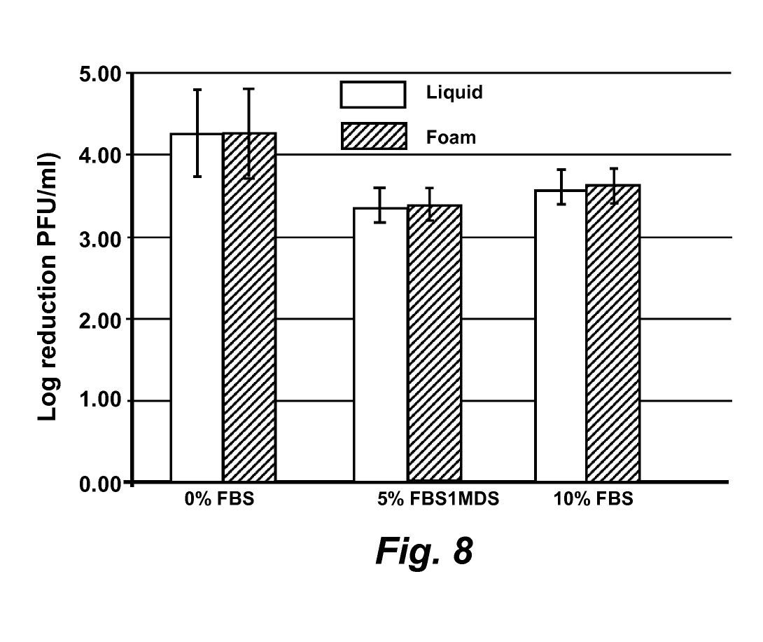

On stainless steel coupons, the 2% levulinic acid plus 1% sodium dodecyl sulfate and 5% levulinic acid plus 2% sodium dodecyl sulfate liquid and foam treatments resulted in less than 3 log reductions in viable MNV after 5 min of treatment. After 1 min of treatment, the liquid and foam sanitizer produced variable results for efficacy against MNV, as shown in FIG. 8. However, the 5% levulinic acid plus 1% sodium dodecyl sulfate and the 5% levulinic acid plus 2% sodium dodecyl sulfate effectively inactivated MNV on the surface of stainless steel coupons, resulting in 3.38-4.25 log reduction in viable MNV after 5 min for both liquid and foam treatments. The addition of increasing amounts of proteinaceous material (FBS) up to 20% did not impact the efficacy of the liquid or foam sanitizer. MNV removal from stainless steel coupons by water remained low (<2.7 log removal).

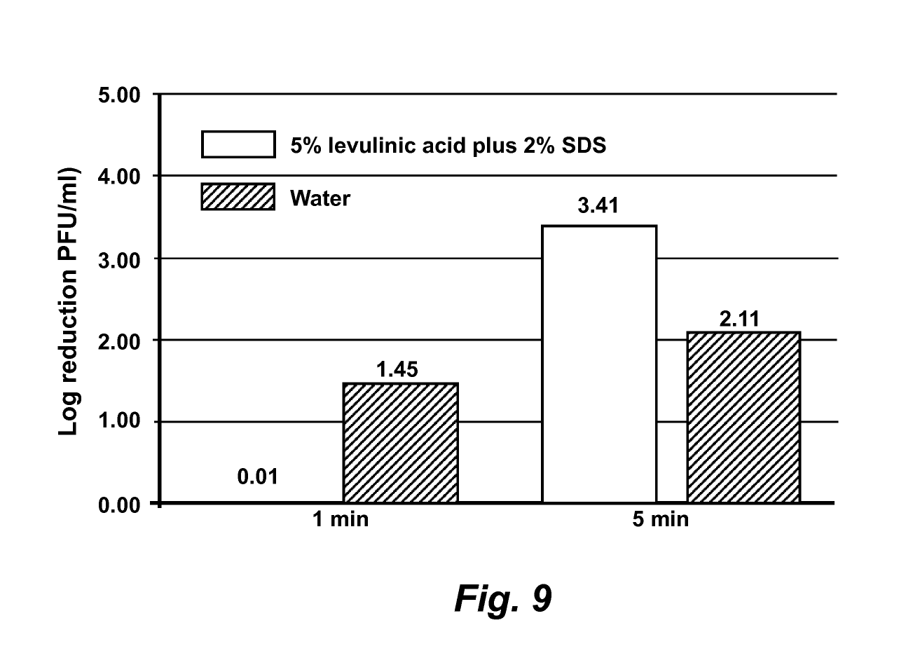

For grapes, MNV was not inactivated at 1 min, but was reduced by 3.41 log after the 5 min treatment with 5% levulinic acid plus 2% sodium dodecyl sulfate. MNV removal from the grape surface by water was only 2.11 log after 5 min, as shown in FIG. 9.

### Example 10

Bacteria cultivation and plaque assay: Five serovars of Salmonella enterica, (Typhimurium, Enteritidis, Gaminara, Agona, and Montevideo) were inoculated separately in 10 mL of tryptic soy broth (Difco, Franklin Lakes, N.J.). Cultures were incubated at 37° C. and transferred twice to fresh vials of TSB, 24 hours apart. For each strain, 5 ml of inoculated broth was transferred to a conical 50 ml tube and centrifuged at 8,000×g at 4° C. for 10 minutes. The supernatant was removed and replaced with 25 ml of 0.1M phosphate buffered saline before the tube was vortexed for 1 minute to resuspend the pellet.

To determine the infectious titer of Salmonella, 1:10 dilutions of the stock were made in 0.1% wt/vol peptone water to a final concentration of 1×10⁶ of the original stock. A wasp spiral plater (Don Whitley Scientific, West Yorkshire, UK) was used to inoculate 0.1 ml of diluted stock on 100 mm diameter media plates of both tryptic soy agar (Difco) plates as a general media and XLT4 agar (Difco) as a selective media. Plates were incubated at 37° C. for 24 hours. Counting of colonies was performed on a colony plate counter (Acolyte, Don Whitley Scientific, West Yorkshire, UK). Plates with 25-250 colonies were used to determine the bacteria titer in colony forming units (CFU).

### Example 11

Protocol for removal for pathogen inactivation on stainless steel and gloves: Sterile stainless steel coupons (4 cm×2.5 cm) were inoculated with 50 μl of MNV or HAV partially purified cell culture lysate (approximately 7×10⁶ PFU/ml stock) spread over the coupon with the pipette tip and 100 μl of Salmonella stock (approximately 6×10⁸ PFU/ml stock) as ten 10 μl inoculations before allowing to dry in a BSC-2 for 40 min at 21° C. or until visibly dry.

Coupons were placed on an electronic scale, sterilized by exposure to UV light for 15 minutes. For each repetition, 1 or 5 wiping motions was made over the inoculated area using a gloved hand at 50±5 gm pressure. All wipes were cut into 1.5×1.5 cm squares and autoclaved at 121° C. and 16 psi for 30 minutes before use.

Wipes tested included water filters containing nano-alumina fibers (NANOCERAM™, Sanford, Fla.), charge modified glass fibers (VIROSORB™ 1MDS, Cuno, Meriden, Conn.), cellulose filters (Millipore, Billerica, Mass.) and (Whatman, Kent, UK).

For wet wipes, each wipe was immersed into a 50 ml conical tube containing the desired sanitizer (2% levulinic acid plus 1% sodium dodecyl sulfate, 5% levulinic acid plus 2% sodium dodecyl sulfate, 5% levulinic acid plus 1% sodium dodecyl sulfate or 5% levulinic acid plus 2% sodium dodecyl sulfate) or water, removed with a pipet tip and gently squeezed by pressing the wipe against the side of the tube with the tip to remove excess moisture.

To mimic hand sanitation procedures, the index fingers of latex gloves decontaminated by UV light for 15 minutes were placed over a sterile 15 ml conical tube. The finger tip was inoculated with 10 μl of MNV stock (approximately 1×10⁶) and dried in a BSC-2 for 30 mins or until visibly dry. The tube was placed on an electronic scale and secured with tape. Wiping was performed as previously described.

### Example 12

Recovery of pathogen: Post-wipe, coupons inoculated with MNV or HAV were immediately placed in a 50 ml tube containing 10 ml 0.1M PBS with 1M NaCl and vortexed for 30 secs to neutralize the sanitizer. The eluate was plated in 1:10 dilutions on the cell culture using previously described methods. For Salmonella, coupons were placed in a stomacher bag (Seward, West Sussex, UK) containing 50 ml 0.1M PBS with 0.02% vol/vol TWEEN™ 80. Using a Seward stomacher 400 model (Seward, West Sussex, UK), the bags were stomached at 230 rpm for 1 minute. Homogenate was plated in 1:10 dilutions on TSA and XLT4 media as previously described. Duplicate cell culture plates for all pathogens were averaged and used to calculate titer after treatment. For a positive control, an inoculated coupon or latex glove finger was dried and pathogen was recovered without any wipe treatment. Negative controls, stainless steel without inoculation, were performed in duplicate and included with each experimental trial. Log reduction of pathogen was calculated as follows: log value of pfu recovered from wiped coupon or glove subtracted from the log value recovered from positive control.

### Example 13

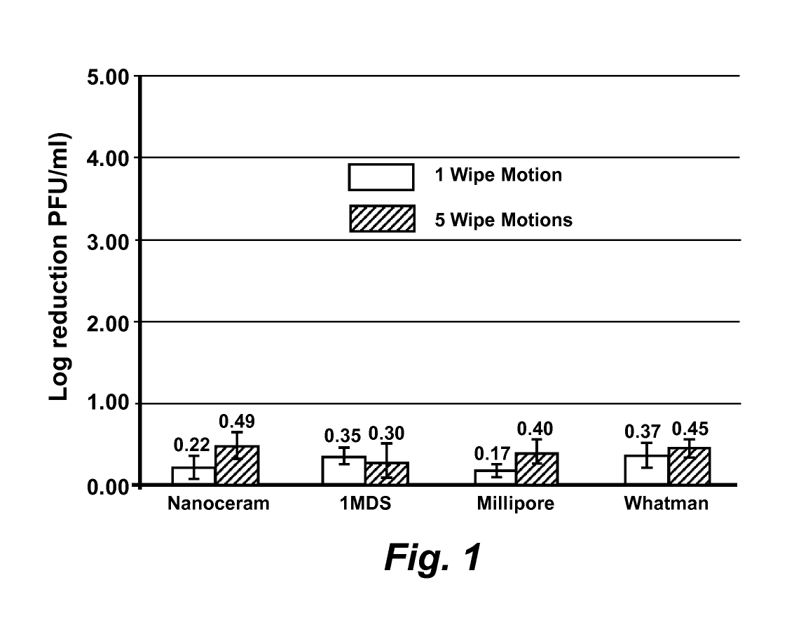

For MNV, dry wipes yielded <0.5 log reduction for all wipes regardless of surface charge (FIG. 1). While positively-charged NANOCERAM™ wipes had the highest log reduction value (0.49) after 5 wiping motions, no differences were found between the different types of wipes or the number of wiping motions (p>0.05). For wipes soaked in water using 5 wiping motions, both types of positively charged wipes were capable of more MNV removal than dry wipes (1.30-1.33 avg. log reduction; p≦0.03), but no difference was observed for neutrally charged wipes (1.13 avg. log reduction; p=0.06).

Wiping with positively charged wipes (5 wiping motions) soaked in different combinations of levulinic acid plus sodium dodecyl sulfate resulted in greater log reductions of MNV than did wiping with wipes soaked in water (p>0.05) for all treatments except 5% levulinic acid plus 2% sodium dodecyl sulfate. Using 5 swiping motions, average log reductions were 2.29, 2.78, and 2.08 for NANOCERAM™, 1MDS and Whatman at the 2% levulinic acid plus 1% sodium dodecyl sulfate level, and 2.71, 2.42, and 2.08 for the same wipes at the 5% Levulinic acid plus 2% sodium dodecyl sulfate level.

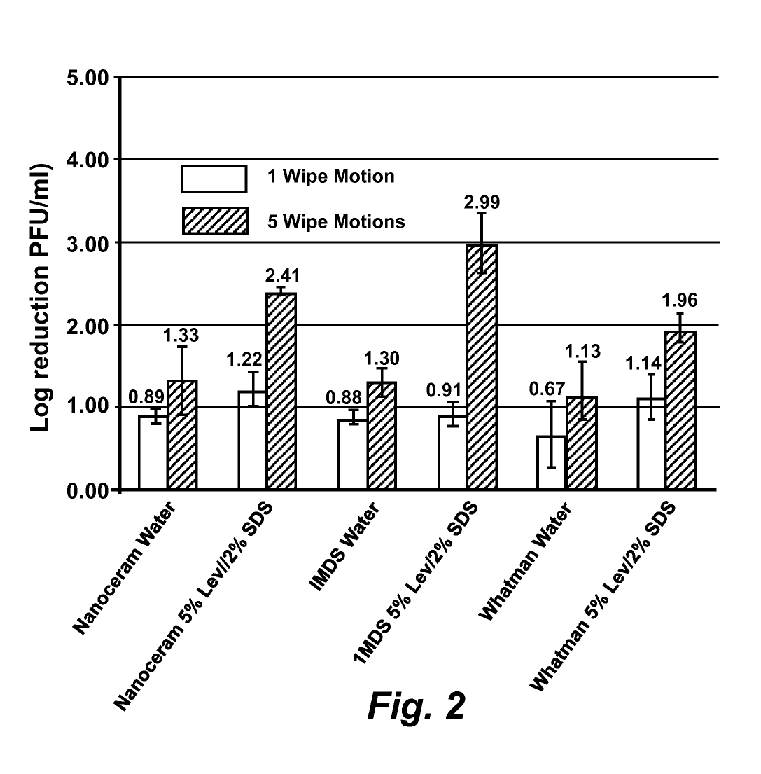

The greatest log reduction for MNV using wet wipes yielded as high as a 2.99 log reduction using the 1 MDS wipe in combination with a 5% Levulinic acid/2% sodium dodecyl sulfate solution (as shown in FIG. 2) which was significantly greater than using the 1 MDS wipe with water (1.30 average log reduction). No differences in MNV log reduction were observed between treatment with 5% levulinic acid plus 1% sodium dodecyl sulfate or 5% levulinic acid plus 2% sodium dodecyl sulfate. One wiping motion was less effective than 5 for nearly all experiments involving wet wipes.

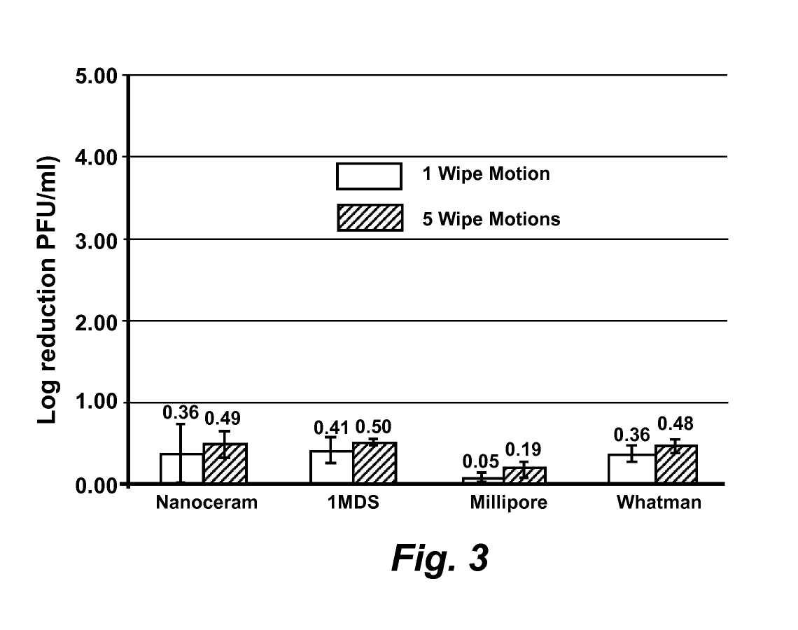

Dry wipes tested with HAV yielded a maximum of 0.5 logs reduced using the positively charged 1 MDS wipe with 5 wiping motions (as shown in FIG. 3). While a positive charge did not improve HAV removal from stainless steel surfaces, both positive and neutral charge wipes were more effective than negatively charged (Millipore) wipes (p=0.002) using 5 swiping motions.

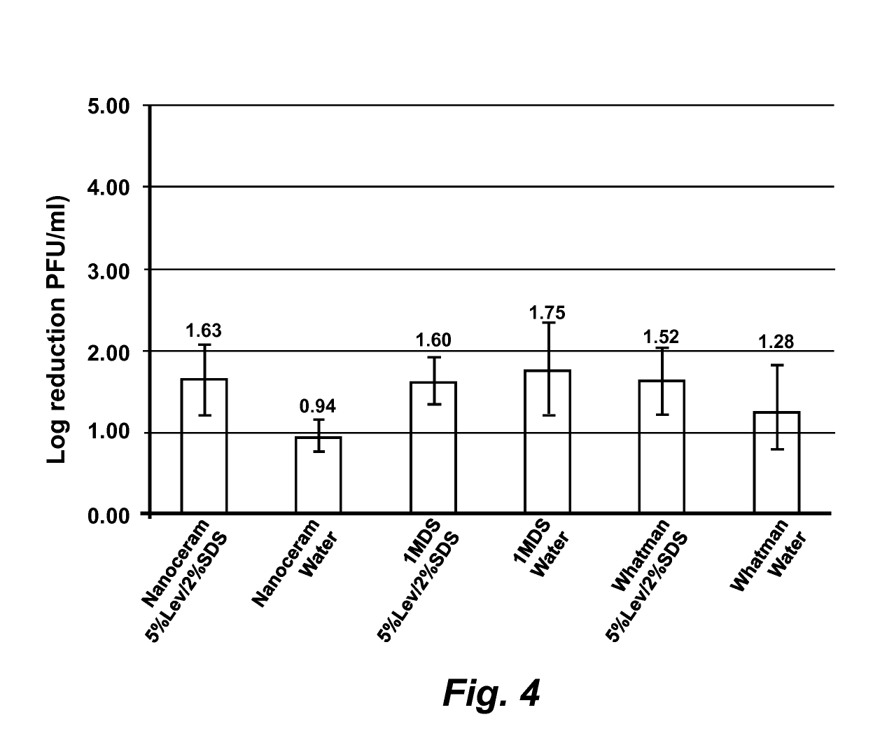

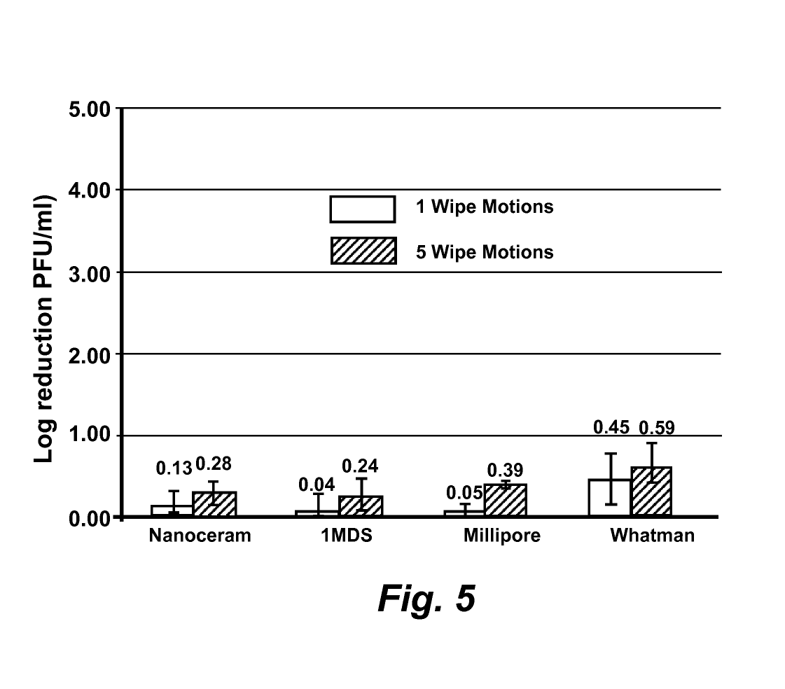

For wipes soaked in water or 5% levulinic acid plus sodium dodecyl sulfate, all tests resulted in <1.5 average log reduction regardless of the ionic charge of the wipes, as shown in FIG. 4. Wipes soaked in levulinic acid plus sodium dodecyl sulfate did not provide significantly greater log reduction in PFU/ml of HAV than was achieved using water (p≧0.05).

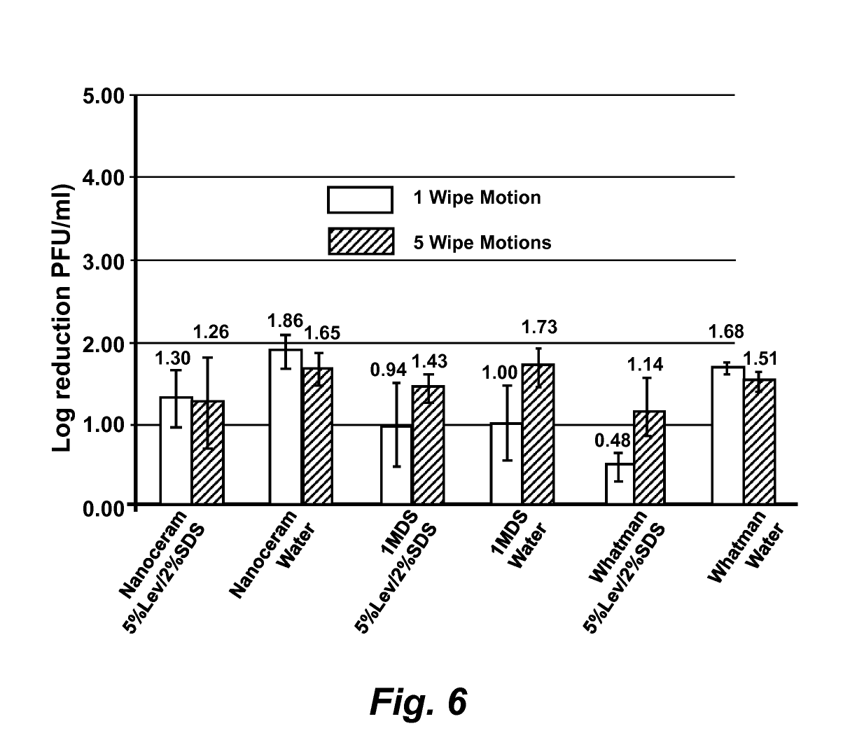

Neutral charge, dry, Whatman wipes yielded a maximum reduction of 0.59 logs using 5 wiping motions for Salmonella enterica, as shown in FIG. 6. However, this value was not significantly greater than the log reduction achieved by the positive or negative charge wipes (p≧0.21). For wet wipes, the positively charged NANOCERAM™ and 1 MDS wipes did not show a significant increase over the neutral Whatman wipe, as shown in FIG. 6). All tests yielded <2 log reduction regardless of the charge or number of wipes made. Comparison among NANOCERAM™ and 1 MDS vs. Whatman wipes soaked in water of Lev plus sodium dodecyl sulfate showed no significant difference in log reduction (p≧0.05). Comparison of 1 or 5 wiping motions yielded a significant difference using Whatman wipes with water for both 1 and 5 swiping motions (p≦0.02).

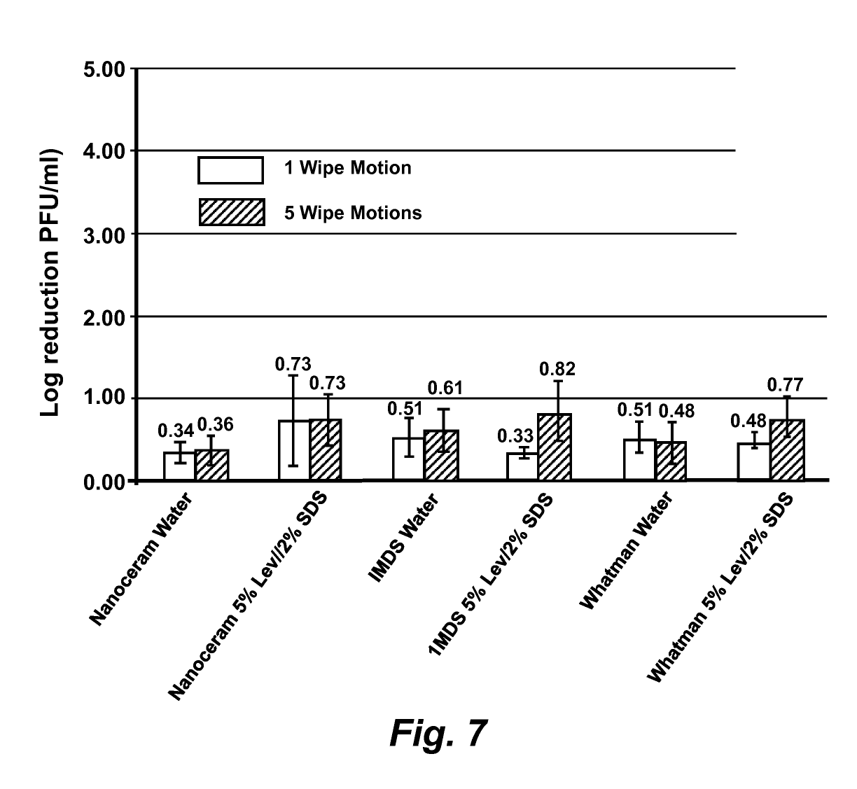

Positive charge NANOCERAM™ and 1MDS and neutral charge Whatman wipes all yielded less than 1 log reduction in PFU/ml of MNV on latex gloved hands, as shown in FIG. 7. No significant differences were observed regardless of the charge of the wipe, treatment of the wipe or number of swiping motions (p≧0.05).

### Example 14

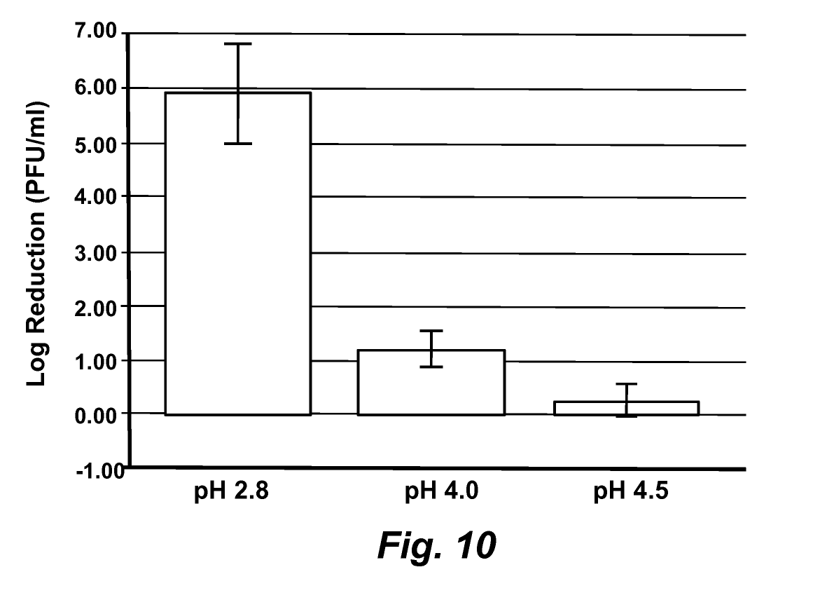

Inactivation of MNV-1 by levulinic acid plus SDS solution is pH dependent: The 5% levulinic acid plus 2% SDS sanitizer at its unadjusted pH of 2.8 effectively inactivated MNV-1 in solution after a 1-min contact time. In every trial at a pH of 2.8, MNV-1 was completely inactivated. Thus, the average log reduction for the sanitizer at a pH of 2.8 is equal to the average neutralization control recovery for this set of experiments, which was 5.60 log PFU/ml. Increasing the pH of the sanitizer to 4.0 and 4.5 weakened its efficacy against MNV-1. At a pH of 4.0, the average log reduction was only 1.23 log PFU/ml, and at a pH of 4.5, there was only a 0.28-log PFU/ml reduction of the virus, as shown in FIG. 10.

### Example 15

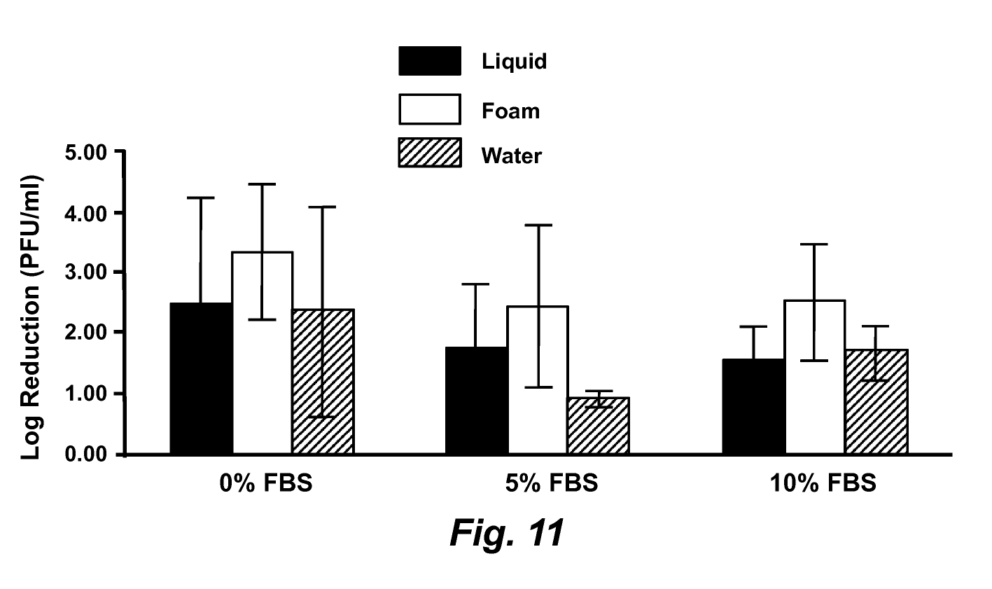

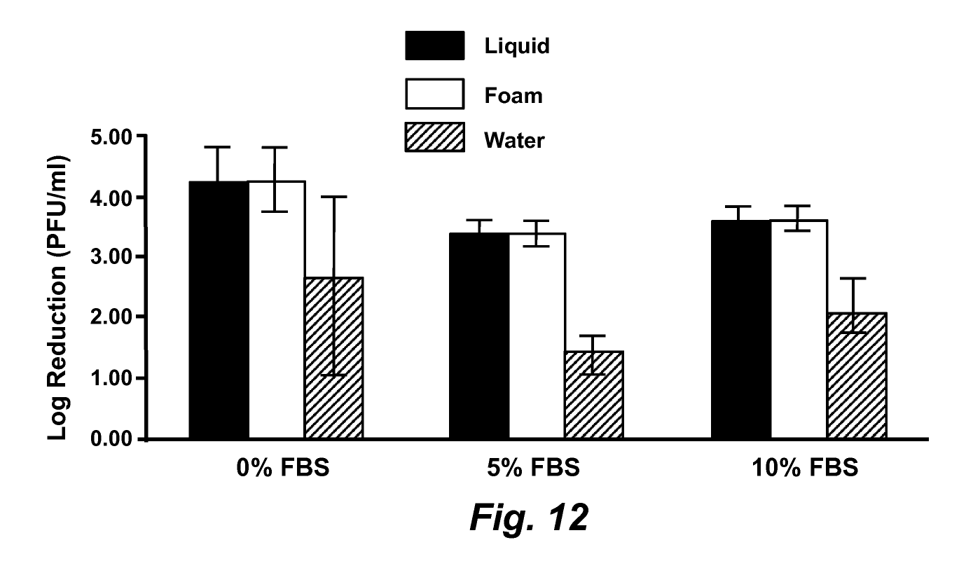

Sanitizer is effective against MNV-1 on stainless steel surfaces and in the presence of organic material: Exposure of stainless steel surfaces inoculated with either a purified MNV-1 stock or MNV-1 suspended in 5% or 10% organic matter (FBS) to either a liquid (50 ml) or foam (25 ml) solution containing 5% levulinic acid plus 2% SDS resulted in reductions of infectious MNV-1. After a 1-min exposure, purified MNV-1 was reduced by 2.51 log PFU/ml or 3.35 log PFU/ml when exposed to the sanitizer in the liquid or foam state, respectively, as shown in FIG. 11. However, similar levels of virus reduction were observed after stainless steel coupons were treated with water for 1 min. Significant reductions in efficacy were not observed with increasing the FBS concentration in the virus stock to 5% or 10%, as shown in FIG. 11. After 5 min of exposure, MNV-1 average log PFU/ml reductions ranged from 3.38-4.25 infectious MNV-1 for both liquid and foam treatments of 5% levulinic acid plus 2% SDS, as shown in FIG. 12. Removal of MNV-1 from stainless steel coupons by water was similar to the levels observed after 1-min exposure times; average log PFU/ml reductions ranged from 1.43 to 2.63, as shown in FIG. 12. The addition of increasing amounts of organic material (FBS) in the virus inoculum (up to 10%) did not affect the efficacy of the liquid or foam sanitizer or virus removal by water. MNV-1 removal from stainless steel surfaces by water was significantly lower than virus log reductions obtained using the liquid or foam sanitizer (p≦0.05).

### Example 16

Bactericidal Efficacy of the Organic Acid/SDS Compositions: Five isolates of E. coli O157:H7, including 932 (human isolate), E009 (beef isolate), E0018 (cattle isolate), E0122 (cattle isolate), E0139 (deer jerky isolate); and five isolates of Salmonella typhimurium DT104, including three cattle isolates and two meat isolates; and five isolates of Salmonella enteritidis, including 564-88 (food isolate), 193-88 (human isolate), E39 (egg isolate), 460-88 (egg isolate) and 457-88 (poultry isolate); and five isolates of L. monocytogenes, including LM101 (serotype 4b, salami isolate), LM 112 (serotype 4b, salami isolate), LM113 (serotype 4b, pepperoni isolate), LM9666 (serotype ½c, human isolate), and LM5779 (serotype ½ c, cheese isolate); and one isolate of Yersinia pestis (A1122) were used. Each Salmonella and E. coli O157:H7 strain was grown in tryptic soy broth (TSB) at 37° C. for 18 h then washed in 0.1M phosphate buffered saline pH 7.2. Approximately equal cell numbers of each of the five strains were combined and used as a 5-strain mixture with cell numbers being adjusted according to the experimental design. Bacterial cell numbers were confirmed by serial dilutions (1:10) in 0.1% peptone and a volume of 0.1 ml from each dilution tube was plated on tryptic soy agar (TSA), XLD agar, and Sorbitol MacConkey agar (SMA), incubated at 37° C. for 24 h, and colonies were counted.

Acetic acid, caprylic acid, lactic acid, levulinic acid and sodium dodecyl sulfate (SDS) were tested alone or as a combination at different concentrations and temperatures (8° C. or 21° C.) for their killing effect on S. enteritidis, S. typhimurium, and E. coli O157:H7 in water or chicken skin contaminated with chicken feces or feathers.

Feces from 5 different chickens and used as a mixture. Feathers were obtained from a slaughterhouse. Chicken and poultry wings were purchased from a slaughter plant or local retail store and skin was separated immediately before use. Only Salmonella-negative chicken feces, feather, skin, or poultry wing samples were used for the experiments. A volume of 10 ml of deionized water and 1.0 g feces, or feathers, or a piece of skin (5×5 cm²) was added to a Whirl-Pak bag. Each bag of feces, feather, or skin sample was pummeled in a stomacher blender at 150 rpm for 1 min. The bag of poultry wing was massaged by hands for 1 min. The fluid was serially (1:10) diluted in 0.1% peptone and 0.1 ml from each dilution tube was plated in duplicate on XLD plates to determine if these samples were contaminated with salmonellae. Enumeration of S. enteritidis, S. typhimurium DT104 and E. coli O157:H7: At each sampling time, 1.0 ml of the treated bacterial suspension was mixed with 9.0 ml of neutralizing buffer or PBS (depending on the pH). The solution was serially (1:10) diluted in 0.1% peptone water and 0.1 ml of each dilution was surface-plated onto TSA and XLD, or TSA and XLD containing ampicillin (32 mg/ml), tetracycline (16 mg/ml) and streptomycin (64 mg/ml) (TSA+, XLD+), or TSA and Sorbitol MacConkey agar plates in duplicate. The plates were incubated at 37° C. for 48 h. Colonies typical of Salmonella or E. coli O157:H7 were randomly picked from plates with the highest dilution for confirmation of Salmonella or E. coli by biochemical tests and for confirmation of serotyping by latex agglutination assay. When Salmonella or E. coli O157:H7 were not detected by direct plating, a selective enrichment in universal pre-enrichment broth (UPB) was performed by incubating 25 ml of treatment suspension in a 500-ml flask containing 225 ml of UPB for 24 h at 37° C. Following pre-enrichment, 1 ml was transferred to 10 ml of selenite cystine broth and incubated for 24 h at 37° C. Following incubation, a 10-μl loopful from the broth tube was plated in duplicate onto XLD plates, and incubated for 24 h at 37° C.

Colonies with typical Salmonella spp. morphology were selected and transferred one more time on XLD plates and incubated for 24 h at 37° C. All presumptive Salmonella isolates were tested by the Salmonella latex agglutination assay. Isolates positive for Salmonella by the latex agglutination assay were tested with the API 20E assay for biochemical characteristics for the identification of Salmonella. Studies with all chemical treatments were done in duplicate or triplicate, two replicates were plated per sample and results were reported as means.

Determination of Salmonella inactivation in water contaminated with chicken feathers or feces: The protocols used were the same as described previously (Zhao, et al. 2006), with minor modifications. Chicken feathers or feces were weighed and added into a glass beaker containing chemicals to be determined according to different ratios (w/v) in a glass beaker and mixed by a magnetic bar with agitation at 150 rpm. A 5-strain mixture of S. enteritidis was added. A volume of 1 ml sample was removed and serially diluted (1:10) in PBS. The aerobic bacterial and Salmonella counts were determined according to the procedures we described above.
Results: Determination of Salmonella inactivation in water with 0.1 to 2.0% by weight levulinic acid revealed about a 1-log CFU/ml reduction. Its killing effect was greater when the levulinic acid concentration was increased to 3.0% by weight, resulting in a 3.4-log Salmonella/ml reduction when in contact for 30 minutes (Table 6). Treatments of 0.5% by weight acetic acid and 0.5% by weight lactic acid for 30 minutes reduced Salmonella cell numbers by 0.7- and 2.0-log CFU/ml, respectively. A treatment of 0.05% by weight SDS for 30 minutes did not reduce Salmonella cell numbers (Table 6).

All the combinations of organic acids evaluated in combination with 0.03-0.05% by weight SDS were effective, at different degrees, in killing Salmonella, with the population of Salmonella quickly reduced from 10⁷ CFU/ml to undetectable (enrichment-negative) with a contact time of 5-10 seconds (see Table 6).

Neither levulinic acid at 0.5% by weight nor SDS at 0.05% by weight when applied individually provided a significant killing effect on either E. coli O157:H7 or S. typhimurium DT 104; however, the combination of levulinic acid and SDS at these concentrations reduced E. coli O157:H7 and S. typhimurium cell numbers by 7 log CFU/ml within 1 min (see Tables 7 and 8).

The levulinic acid and SDS treatment to kill S. enteritidis was further tested in water containing chicken feathers or feces. Results revealed that feather contamination did not reduce the killing effect of that treatment, whereas the presence of chicken feces did. S. enteritidis was reduced from 7.6 log CFU/ml to 1.2 log CFU/ml in chicken feces contaminated water after 2 min exposure, but was not detected (7.6 log CFU/ml reduction) after 5 min (P<0.05; Table 9). Greater concentrations of levulinic acid and SDS were more effective in killing Salmonella, even in water heavily contaminated with chicken feces (1 part feces: 20 parts water; wt/v) (Table 9).

Aerobic bacteria counts in water contaminated with chicken feces at a ratio of 1:100 (w/v) were reduced by >4.0 log CFU/ml after treatment with 1% by weight levulinic acid and 0.1% by weight SDS for 2 min. The antimicrobial effect was increased to ca. 5.5 log CFU/ml reduction in water contaminated with chicken feces at a ratio of 1:20 (w/v) when the chemical concentrations were increased to 3% by weight levulinic acid plus 2.0% by weight SDS for 2 min (Table 10).

In one embodiment the chemical combination comprises 45 mM levulinic acid and 1.73 mM SDS, which can rapidly (within 8 seconds) kill up to 7 log of pathogens, including Yersinia pestis, Salmonella enteritidis, S. typhimurium DT104, Listeria monocytogenes, and Escherichia coli O157:H7. This chemical combination is stable at room temperature and environmentally friendly. There is no apparent organoleptic difference between fresh produce treated with this chemical solution for up to 60 mins and fresh produce treated with water or without treatment.

TABLE 6
Reduction of S. enteritidis in water treated
with organic acids and SDS at 21° C.

| | S. enteritidis counts | | | | | |
|---|---|---|---|---|---|---|
| | (log CFU/ml) at mins: | | | | | |
| Chemical Treatment | 0 | 2 | 5 | 10 | 20 | 30 |
| S. enteritidis only (pH 6.7) (Control) | 7.2 | 7.0 | 7.1 | 7.2 | 7.0 | 7.2 |
| 0.1% levulinic acid (pH 2.5) | 7.1 | 7.1 | 6.9 | 7.0 | 6.9 | 6.9 |
| 0.5% levulinic acid (pH 2.6) | 7.1 | 6.8 | 6.9 | 6.9 | 6.6 | 6.7 |
| 1.0% levulinic acid (pH 2.9) | 6.9 | 6.7 | 6.8 | 6.9 | 6.9 | 6.7 |
| 1.5% levulinic acid (pH 2.8) | 6.7 | 6.7 | 6.8 | 6.7 | 6.4 | 6.5 |
| 2.0% levulinic acid (pH 2.8) | 6.7 | 6.7 | 6.7 | 6.8 | 6.5 | 6.0 |
| 2.5% levulinic acid (pH 2.6) | 6.9 | 6.8 | 6.9 | 6.4 | 5.8 | 4.8 |
| 3.0% levulinic acid (pH 2.7) | 6.6 | 6.8 | 6.5 | 6.2 | 5.1 | 3.8 |
| 0.5% acetic acid (pH 3.1) | 7.1 | 7.0 | 6.8 | 6.7 | 6.6 | 6.5 |
| 0.5% lactic acid (pH 2.6) | 6.5 | 6.1 | 5.9 | 5.8 | 5.5 | 5.2 |
| 0.05% sodium dodecyl sulfate (pH 4.4) | 7.1 | 7.0 | 7.2 | 7.1 | 7.2 | 7.1 |
| 0.3% levulinic acid + 0.05% SDS (pH 3.1) | −^{a} | − | − | − | − | − |
| 0.4% levulinic acid + 0.05% SDS (pH 2.9) | − | − | − | − | − | − |
| 0.5% levulinic acid + 0.05% SDS (pH 3.0) | − | − | − | − | − | − |
| 0.5% levulinic acid + 0.03% SDS (pH 3.0) | − | − | − | − | − | − |
| 0.05% caprylic acid + 0.03% SDS (pH 3.4) | − | − | − | − | − | − |
| 0.05% caprylic acid + 0.05% SDS (pH 3.2) | − | − | − | − | − | − |
| 0.5% acetic acid + 0.05% SDS (pH 3.0) | − | − | − | − | − | − |
| 0.5% lactic acid + 0.05% SDS (pH 2.5) | − | − | − | − | − | − |

^{a}−, negative by enrichment culture.

TABLE 7
Reduction of E. coli O157:H7 in water treated
with levulinic acid and SDS at 21° C.

| | E. coli O157:H7 counts | | | | | | | |
|---|---|---|---|---|---|---|---|---|
| | (log CFU/ml) at min: | | | | | | | |
| Chemical Treatment | 0 | 1 | 2 | 5 | 10 | 20 | 30 | 60 |
| E. coli O157:H7 only (Control) | 7.1 | 7.2 | 7.0 | 7.2 | 7.1 | 7.1 | 7.2 | 7.2 |
| 0.5% levulinic acid-(pH 3.0) | 7.0 | 6.7 | 6.8 | 6.7 | 6.9 | 6.8 | 6.8 | 6.4 |
| 0.05% SDS-(pH 7.0) | 7.1 | 6.9 | 7.1 | 7.0 | 6.9 | 6.9 | 7.1 | 7.0 |
| 0.5% levulinic acid + | ^{a}− | − | − | − | − | − | − | − |
| 0.05% SDS-(pH 3.0) | | | | | | | | |

^{a}−, negative by enrichment culture

TABLE 8
Reduction of S. typhimurium DT 104 in water treated
with levulinic acid + SDS at 21° C.

| | S. typhimurium DT 104 counts | | | | | | | |
|---|---|---|---|---|---|---|---|---|
| | (log CFU/ml) at min: | | | | | | | |
| Chemical Treatment | 0^{a} | 1 | 2 | 5 | 10 | 20 | 30 | 60 |
| S. typhimurium only (Control) | 6.9 | 7.0 | 7.0 | 7.0 | 7.0 | 6.9 | 7.0 | 7.0 |
| 0.5% levulinic acid (pH 3.0) | 6.8 | 6.7 | 6.6 | 6.5 | 6.7 | 6.6 | 6.4 | 5.9 |
| 0.05% SDS (pH 7.0) | 7.0 | 7.0 | 6.8 | 6.9 | 6.8 | 6.9 | 6.9 | 6.9 |
| 0.5% levulinic acid + | +^{a} | −^{b} | − | − | − | − | − | − |
| 0.05% SDS (pH 3.0) | | | | | | | | |

^{a}+, positive by enrichment (minimum detection level is 0.7 log CFU/ml)
^{b}−, negative by enrichment culture

TABLE 9
S. enteritidis counts for treatment of levulinic acid plus
SDS in water containing chicken feathers or feces at 21° C.

| | S. enteritidis counts (log CFU/ml) at min: | | | | | |
|---|---|---|---|---|---|---|
| Treatment | 0 | 2 | 5 | 10 | 20 | 30 |
| In water containing chicken | | | | | | |
| feathers (1:100, w/v) | | | | | | |
| S. enteritidis (pH 6.7) only | 7.5 | 7.7 | 7.4 | 7.5 | 7.6 | 7.6 |
| 1.0% levulinic acid + | <0.7^{a} | <0.7 | <0.7 | <0.7 | <0.7 | <0.7 |
| 0.1% SDS (pH 3.2) | | | | | | |
| In water containing chicken | | | | | | |
| feces (1:100, w/v) | | | | | | |
| S. enteritidis only (pH 6.8) | 7.6 | 7.5 | 7.5 | 7.6 | 7.5 | 7.6 |
| 1.0% levulinic acid + | 4.9 | 1.2 | <0.7 | <0.7 | <0.7 | <0.7 |
| 0.1% SDS (pH 4.0) | | | | | | |
| In water containing chicken | | | | | | |
| feces (1:20, w/v) | | | | | | |
| S. enteritidis only (pH 6.7) | 7.7 | 7.8 | 7.7 | 7.7 | 7.7 | 7.6 |
| 3.0% levulinic acid + | <0.7 | <0.7 | <0.7 | <0.7 | <0.7 | <0.7 |
| 2.0% SDS (pH 4.0) | | | | | | |

^{a}Minimum detection level by direct plating method

TABLE 10
Aerobic bacteria counts for treatment of levulinic acid
plus SDS in water containing chicken feces at 21° C.

| | Bacteria counts (log CFU/ml) at min: | | | | | |
|---|---|---|---|---|---|---|
| Treatment | 0 | 2 | 5 | 10 | 20 | 30 |
| In water containing chicken | | | | | | |
| feces (1:100, w/v) | | | | | | |
| Aerobic bacteria only | 7.4 | ND^{a} | ND | 7.4 | 7.4 | 7.4 |
| 1.0% levulinic acid + | 5.0 | 3.0 | 2.9 | 2.9 | 2.0 | 2.0 |
| 0.1% SDS (pH 4.0) | | | | | | |
| In water containing chicken | | | | | | |
| feces (1:20, w/v) | | | | | | |
| Aerobic bacteria only | 10.4 | 10.4 | 10.3 | 10.4 | 10.4 | 10.4 |
| 3.0% levulinic acid + | 4.5 | 4.9 | 5.1 | 4.9 | 5.1 | 5.1 |
| 2.0% SDS (pH 4.0) | | | | | | |

^{a}ND, Not determined.

TABLE 11
Effect of a combination with 0.5% levulinic acid and 0.05% SDS, pH 3.1 at 21°
C. on bacterial species (ND = Not Determined; a dash “—” indicates “not detected”)

| | Bacterial counts (log CFU/ml) at min: | | | | | | | |
|---|---|---|---|---|---|---|---|---|
| Bacterial Name | 0^{a} | 1 | 2 | 5 | 10 | 20 | 30 | 60 |
| Klebsiella pneumonia in 0.1M PBS | ND^{b} | ND | ND | 6.5 | ND | ND | ND | 6.6 |
| (Control) | | | | | | | | |
| K. pneumonia in 0.5% levulinic acids + | —^{c} | — | — | — | — | — | — | — |
| 0.05% SDS (pH 3.1) | | | | | | | | |
| Hafinia alvei in 0.1M PBS (control) | ND | ND | ND | 6.9 | ND | ND | ND | 6.9 |
| H. alvei in 0.5% levulinic acids + | — | — | — | — | — | — | — | — |
| 0.05% SDS (pH 3.1) | | | | | | | | |
| Klebsiella oxytoca in 0.1M PBS | ND | ND | ND | 7.2 | ND | ND | ND | 7.1 |
| (Control) | | | | | | | | |
| K. oxytoca in 0.5% levulinic acids + | — | — | — | — | — | — | — | — |
| 0.05% SDS (pH 3.1) | | | | | | | | |
| Proteus hauseri in 0.1M PBS | ND | ND | ND | 7.3 | ND | ND | ND | 7.4 |
| (Control) | | | | | | | | |
| Pr. hauseri in 0.5% levulinic acids + | — | — | — | — | — | — | — | — |
| 0.05% SDS (pH 3.1) | | | | | | | | |
| Serratia marcesens in 0.1M PBS | ND | ND | ND | 7.3 | ND | ND | ND | 7.3 |
| (Control) | | | | | | | | |
| Ser. marcesens in 0.5% levulinic | — | — | — | — | — | — | — | — |
| acids + 0.05% SDS (pH 3.1) | | | | | | | | |
| Shigella flexneri in 0.1M PBS | ND | ND | ND | 7.1 | ND | ND | ND | 7.1 |
| (Control) | | | | | | | | |
| Shi. flexneri in 0.5% levulinic acids + | — | — | — | — | — | — | — | — |
| 0.05% SDS (pH 3.1) | | | | | | | | |
| Shi. sonnei in 0.1M PBS (Control) | ND | ND | ND | 7.3 | ND | ND | ND | 7.3 |
| Shi. sonnei in 0.5% levulinic acids + | — | — | — | — | — | — | — | — |
| 0.05% SDS (pH 3.1) | | | | | | | | |
| Staphylococcus aureus in 0.1M | ND | ND | ND | 6.9 | ND | ND | ND | 6.9 |
| PBS (Control) | | | | | | | | |
| Staph. aureus in 0.5% levulinic | — | — | — | — | — | — | — | — |
| acids + 0.05% SDS (pH 3.1) | | | | | | | | |
| Aerococcus viridans in 0.1M PBS | ND | ND | ND | 6.0 | ND | ND | ND | 6.0 |
| (control) | | | | | | | | |
| Aero. viridans in 0.5% levulinic acids + | — | — | — | — | — | — | — | — |
| 0.05% SDS (pH 3.1) | | | | | | | | |
| Yersinia pseudotubersulosis in 0.1M | ND | ND | ND | 7.0 | ND | ND | ND | 7.0 |
| PBS (control) | | | | | | | | |
| Y. pseudotubersulosis in 0.5% | | | | | | | | |
| levulinic acids + 0.05% SDS | | | | | | | | |
| (pH 3.1) | | | | | | | | |
| E. coli O26:H11 in 0.1M PBS | ND | ND | ND | 7.2 | ND | ND | ND | 7.2 |
| (Control) | | | | | | | | |
| E. coli O26:H11 in 0.5% levulinic | — | — | — | — | — | — | — | — |
| acids + 0.05% SDS (pH 3.1) | | | | | | | | |
| E. coli O111:NM in 0.1M PBS | ND | ND | ND | 7.1 | ND | ND | ND | 7.1 |
| (Control) | | | | | | | | |
| E. coli O111:NM in 0.5% levulinic | — | — | — | — | — | — | — | — |
| acids + 0.05% SDS (pH 3.1) | | | | | | | | |
| Vibro chloerae in 0.1M PBS | ND | 5.1 | 5.0 | ND | ND | ND | 4.2 | ND |
| (control) | | | | | | | | |
| V. chloerae in 0.5% levulinic acids + | — | — | — | — | — | — | — | — |
| 0.05% SDS (pH 3.1) | | | | | | | | |
| Campylobacter jejuni in 0.1M PBS | 8.2 | 8.3 | 8.1 | 8.0 | 8.4 | 8.1 | 8.2 | 8.4 |
| (control) | | | | | | | | |
| Camp. jejuni in 0.5% levulinic acids + | <0.7 | <0.7 | <0.7 | <0.7 | <0.7 | <0.7 | <0.7 | <0.7 |
| 0.05% SDS (pH 3.1) | | | | | | | | |

^{a}Initial inoculation level: Hafinia alvei: 1.9 × 10⁸ CFU/ml; Klebsiella oxytoca: 2.1 × 10⁹ CFU/ml; Proteus hauseri: 1.3 × 10⁹ CFU/ml; Serratia marcesens: 1.2 × 10⁹ CFU/ml; Shigella flexneri: 1.1 × 10⁹ CFU/ml; Shigella sonnei: 1.3 × 10⁹ CFU/ml; Staphylococcus aureus: 1.9 × 10⁸ CFU/ml; Aerococcus virians: 1.0 × 10⁸ CFU/ml; Yersinia pseudotuberculosis: 1.0 × 10⁹ CFU/ml; E. coli O26:H11: 1.2 × 10⁹ CFU/ml; E. coli O111:NM: 1.1 × 10⁹; Vibrio cholerae: 1.2 × 10⁶ CFU/ml; Campylobacter jejuni: 1.2 × 10¹⁰ CFU/ml.
^{b}The actual time 0 was delayed by 5 to 10 seconds due to time for sample processing.
^{c}ND, not determined.
^{d}Negative by direct plating and enrichment culture.

### Example 17

Efficacy of the Organic Acid/SDS Compositions against L. monocytogenes: The efficacy of the antibacterial compositions disclosed herein was tested against Listeria monocytogenes using the same assay and procedures disclosed in Example 1. The results are indicated in Table 12.

TABLE 12
Reduction of L. monocytogenes by different concentrations of
levulinic acid and SDS individually and in combination at 21° C.

| | L. monocytogenes counts (log CFU/ml) at min: | | | | | |
|---|---|---|---|---|---|---|
| Chemical Treatment | 0^{a} | 2 | 5 | 10 | 20 | 30 |
| 0.5% levulinic acid (pH 3.1) | 6.7 ± 0.2 | 6.7 ± 0.1 | 6.8 ± 0.3 | 6.9 ± 0.2 | 6.7 ± 0.2 | 6.8 ± 0.2 |
| 1.0% levulinic acid (pH 3.0) | 6.8 ± 0.3 | 6.7 ± 0.2 | 6.6 ± 0.3 | 6.6 ± 0.3 | 6.6 ± 0.0 | 6.6 ± 0.3 |
| 1.5% levulinic acid (pH 2.9) | 6.9 ± 0.1 | 6.9 ± 0.2 | 6.9 ± 0.3 | 6.9 ± 0.1 | 6.9 ± 0.3 | 6.8 ± 0.3 |
| 2.0% levulinic acid (pH 2.9) | 6.8 ± 0.3 | 6.8 ± 0.2 | 6.9 ± 0.2 | 6.7 ± 0.2 | 6.9 ± 0.2 | 6.8 ± 0.2 |
| 0.05% sodium dodecyl sulfate | 6.6 ± 0.3 | 6.4 ± 0.1 | 6.0 ± 0.1 | 5.0 ± 0.3 | 3.8 ± 0.2 | 3.3 ± 0.1 |
| (pH 4.8) | | | | | | |
| 0.5% levulinic acid + 0.05% SDS | −^{c} | − | − | − | − | − |
| (pH 3.0) | | | | | | |

^{a}The actual time 0 was delayed by 5 to 10 seconds due to time for sample processing.
^{b}+, Positive by enrichment culture but not by direct plating (minimum detection level is 0.7 log CFU/ml).
^{c}−, Negative by direct plating and enrichment culture.

### Example 18

Reduction of microorganisms by different chemical combination at 21° C.: Different combinations of pharmaceutically acceptable acids in combination with various pharmaceutically acceptable surfactants were tested for their antibacterial properties.

Microorganisms were contacted with the test compositions using the same assay and procedures as disclosed in Example 16. The results obtained by contacting microorganisms with different surfactant/acid combinations are indicated in Tables 13 and 14. As indicated by the following data, particularly Table 14, not all organic acids/surfactant combinations perform equivalently with regards to their efficacy as antimicrobial agents.

TABLE 13
Reduction of microorganisms by different chemical combination at 21° C.

| Chemical treatment | 0^{a} | 1 | 2 | 5 | 10 | 20 | 30 | 60 |
|---|---|---|---|---|---|---|---|---|
| | E. coli O157:H7 counts (log CFU/ml) at min: | | | | | | | |
| E. coli O157:H7 only (Control) | 7.2 | 7.4 | ND^{b} | 7.3 | ND | ND | 7.3 | 7.4 |
| 0.05% SDS to pH 3.0 by 1N HCl | <0.7 | <0.7 | <0.7 | <0.7 | <0.7 | <0.7 | <0.7 | <0.7 |
| | S. enteritidis counts (log CFU/ml) at min: | | | | | | | |
| S. enteritidis only (Control) | 7.2 | 7.1 | ND | 7.2 | ND | ND | 7.4 | 7.3 |
| 0.05% SDS to pH 3.0 by 1N HCl | <0.7 | <0.7 | <0.7 | <0.7 | <0.7 | <0.7 | <0.7 | <0.7 |
| | Y. pestis counts (log CFU/ml) at min: | | | | | | | |
| Y. pestis only (Control) | 6.3 | 6.1 | 6.4 | 6.7 | 6.6 | 6.5 | 6.7 | 6.7 |
| 0.5% Levulinic acid plus | <0.7 | <0.7 | <0.7 | <0.7 | <0.7 | <0.7 | <0.7 | <0.7 |
| 0.05% SDS (pH 3.0) | | | | | | | | |

^{a}The actual time 0 was delayed by 5 to 10 seconds due to time for sample processing.
^{b}ND, not determined.
^{a}The actual time 0 may was delayed by 10 to 20 seconds due to time for sample processing.
^{b}+, Below the minimum detection level by direct plating (<0.7 log CFU/ml), but positive by enrichment culture.

### Example 19

### Treatment of Biofilms with Compositions Comprising an Acid and Surfactant

Preparation of stainless steel coupons: Coupons (4 cm×2.5 cm) composed of different materials, including stainless steel, polyvinyl chloride, nitrile rubber, glass, ultra-high molecular weight polyethylene were washed by a 10 min immersion with agitation (150 rpm) in 1000 ml of an aqueous 2% RBS 35 Detergent Concentrate solution (20 ml of RBS 35 Concentrate per liter of tap water at 50° C.; Pierce, Rockford, Ill.), and rinsed by immersion in 1000 ml of tap water (initial at 50° C.) with agitation (150 rpm) for 25 min. Five additional 1-min immersions with agitation (150 rpm) in 1000 ml of distilled water at ambient temperature were performed. The coupons were dried. The coupons were then individually wrapped and autoclaved at 121° C. for 30 min.

Biofilm formation of S. enteritidis on coupons: For purpose of a well-formatted biofilm of S. enteritidis on the surface of coupons, the coupons were placed individually in a 250-ml flask containing 100 ml tryptic soy broth (TSB) and an inoculum of 1.0 ml ca. 10⁸ CFU of a 5-strain mixture of S. enteritidis was added. The flasks were incubated at 37° C. for 24 h. The coupons then were removed individually and placed on the surface of a layer of paper tower for absorbing the extra fluid of the surface.

The coupons having the formed biofilms were then individually transferred to plates containing 30 ml chemical solution for treatment for 0, 1, 2, 5, 10, 20 min. Following treatment each coupon was placed in a 50-ml centrifuge tube containing 9.0 ml of PBS and 30 glass beads (5 mm). The tubes were agitated by a Vortex for 2 min to suspend the adherent bacteria. The suspended bacteria were serially diluted (1:10) in 0.1% peptone and plated in duplicate on TSA and XLD agar plates for S. enteritidis enumeration. The plates were incubated for 48 h at 37° C. and bacterial colonies counted.

Studies of S. enteritidis attached to the surface of the coupons revealed that the pathogen was eliminated in less than 1 minute by the treatment solution containing 3% levulinic acid plus 2% SDS (Tables 14 and 15).

TABLE 14
Reduction of S. enteritidis on stainless steel
coupons by levulinic acid plus SDS at 21° C.

| Treatment | 0^{a} | 1 | 2 | 5 | 10 | 20 |
|---|---|---|---|---|---|---|
| | S. enteritidis counts (log CFU/cm²) with coupons | | | | | |
| | incubated for 2 h at min: | | | | | |
| PBS (7.2) (Control) | 7.4 | 7.3 | ND^{b} | 7.3 | ND | 7.4 |
| 3% levulinic acid + | <0.7 | <0.7 | <0.7 | <0.7 | <0.7 | <0.7 |
| 2% SDS (pH 2.7) | | | | | | |
| | S. enteritidis counts (log CFU/cm²) with coupons | | | | | |
| | incubated for 4 h at min: | | | | | |
| | 0 | 1 | 2 | 5 | 10 | 20 |
| | <0.7 | <0.7 | <0.7 | <0.7 | <0.7 | <0.7 |
| | S. enteritidis counts (log CFU/cm²) with coupons | | | | | |
| | incubated for 24 h at min: | | | | | |
| | 0 | 1 | 2 | 5 | 10 | 20 |
| | <0.7 | <0.7 | <0.7 | <0.7 | <0.7 | <0.7 |

^{a}The actual time 0 may was delayed by 15 to 25 seconds due to time for sample processing
^{b}Not determined

TABLE 15
Chemical inactivation of S. enteritidis in
biofilm at 21° by 3% levulinic acid plus 2% SDS

| | | S. enteritidis count | | | |
|---|---|---|---|---|---|
| | | (log CFU/cm²) at min: | | | |
| Coupon | Treatment Solution | 0 | 1 | 5 | 10 |
| Stainless | PBS, pH 7.2 | 8.0 | 8.4 | 8.6 | 8.2 |
| | NaClO₂ (500 ppm), pH 2.8 | 7.5 | 5.9 | 5.4 | 6.2 |
| | 3.0% levulinic acid (LV) | <0.7 | <0.7 | <0.7 | <0.7 |
| | plus 2.0% SDS, pH 3.0 | | | | |
| Polyvinyl | PBS | 8.8 | 9.0 | 8.8 | 8.0 |
| chloride | NaClO₂ (500 ppm) | 6.9 | 5.5 | 5.3 | 4.2 |
| | 3.0% LV plus 2.0% SDS | 2.3 | 1.7 | 2.2 | <0.7 |
| Nitrile rubber | PBS | 7.8 | 8.0 | 7.7 | 7.9 |
| | NaClO₂ (500 ppm) | 7.2 | 5.2 | 2.6 | 1.3 |
| | 3.0% LV plus 2.0% SDS | 4.1 | 1.7 | <0.7 | <0.7 |
| Glass | PBS | 8.2 | 8.7 | 8.4 | 8.4 |
| | NaClO₂ (500 ppm) | 6.8 | 3.3 | <0.7 | <0.7 |
| | 3.0% LV plus 2.0% SDS | <0.7 | <0.7 | <0.7 | <0.7 |
| Ultra-high | PBS | 8.4 | 8.4 | 8.4 | 8.4 |
| molecular | NaClO₂ (500 ppm) | 6.8 | 6.1 | <0.7 | <0.7 |
| weight | 3.0% LV plus 2.0% SDS | <0.7 | <0.7 | <0.7 | <0.7 |
| polyethylene | | | | | |

### Example 20

Efficacy of compositions to kill spores of Bacillus anthracis Sterne: For all experiments an equal volume of spore suspension of B. anthracis Sterne (34F₂) was added to 25 ml of reagents A, B, C, D, E, and F in 250-ml flasks. The compositions of reagents are A: 3% levulinic acid plus 2% SDS; B: 2% levulinic acid plus 1% SDS; C, 0.5% levulinic acid plus 0.05% SDS; D: 3% levulinic acid; E: 2% SDS; F: water (control).

Flasks were incubated at 37° C. in a shaker (200 rpm). At each time point 100 μl of sample was transferred into 900 μl water, vortexed, and 100 μl of the dilution spread on Brain Heart Infusion agar plates. Plates were incubated at 37° C. overnight and colonies counted the next morning (approximately 16 hours later).

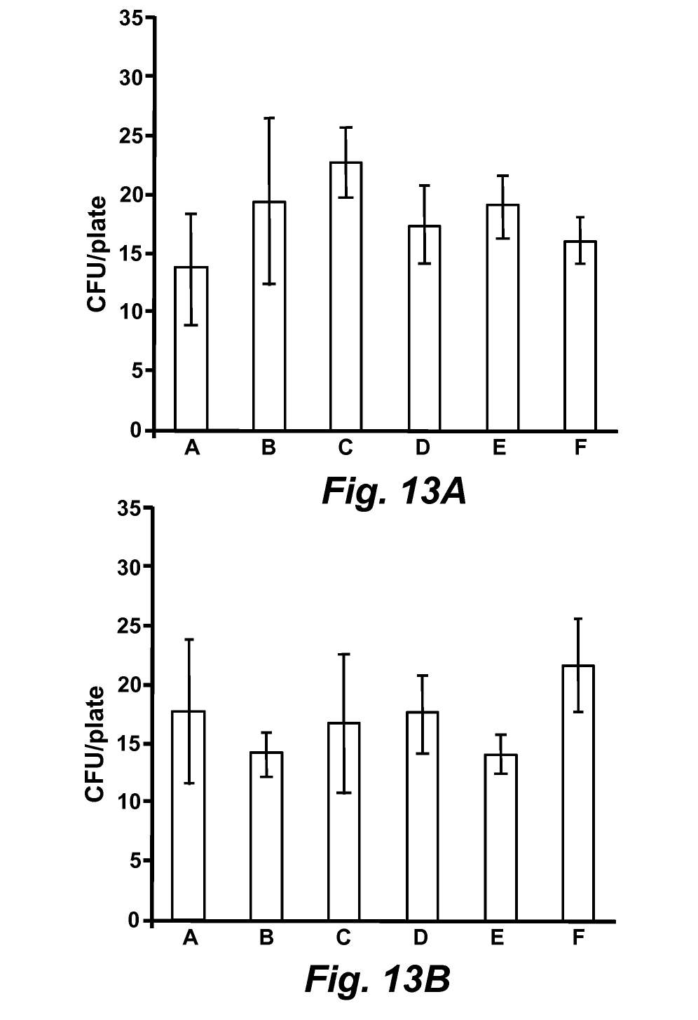

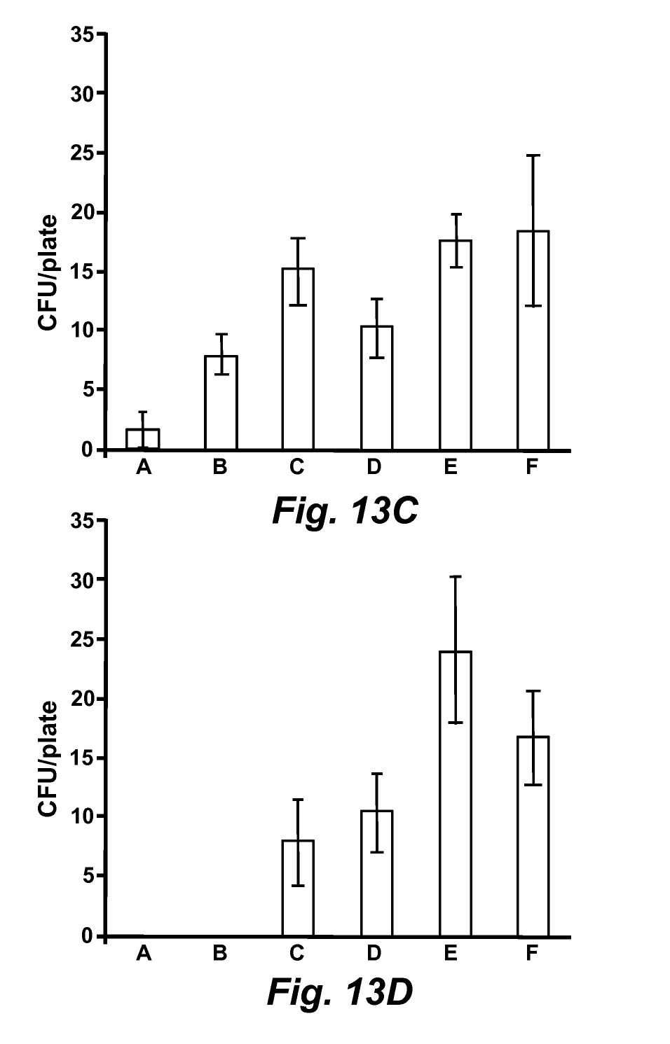

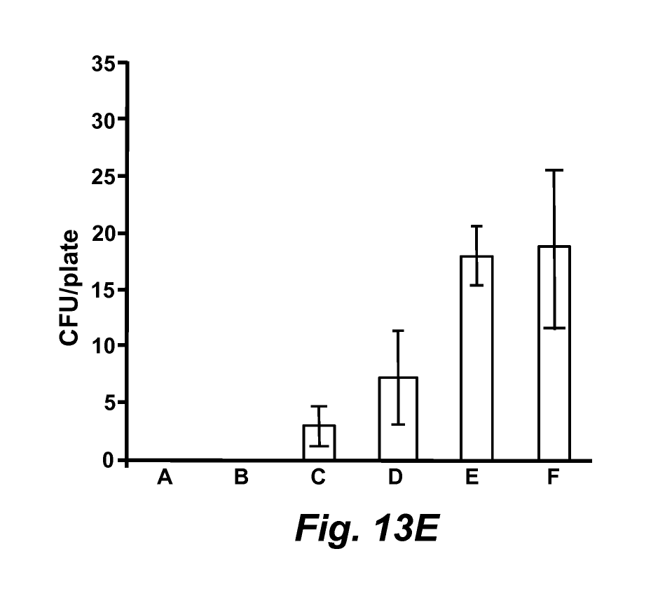

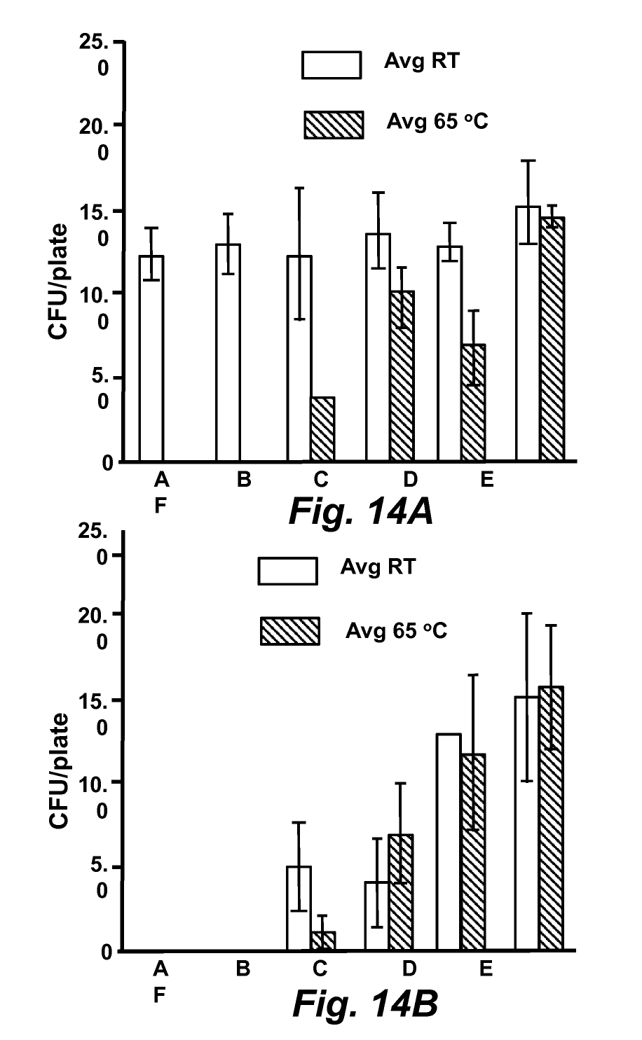

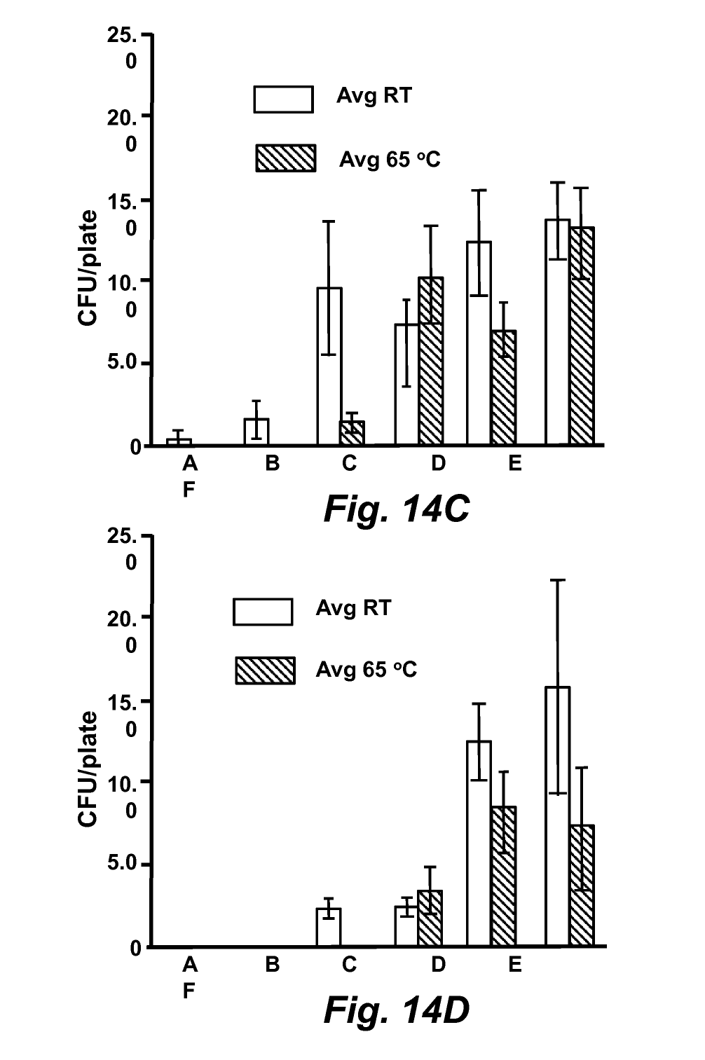

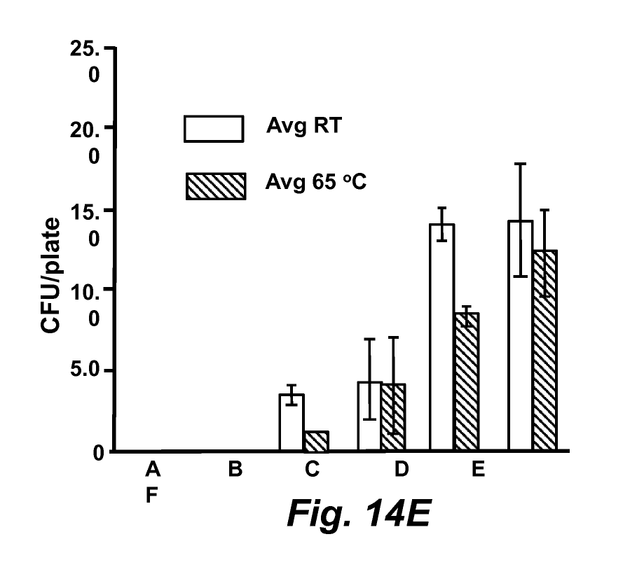

Experiment A3: 250 μl spore suspension (5×10⁴ spores) were added to 25 ml of the reagents. Sampling time points were t=0 (spores were added and after mixing with the reagent, 100 μl of the suspension were removed for enumeration), t=10 min, t=45 min, t=90 min, t=180 min. Average plate counts (FIGS. 13A-13E) are based on counting three plates; error bars indicate +/−one standard deviation.
Experiments A4, A5: In experiment A4, 250 μl spore suspension (5×10⁴ spores) were added to 25 ml of the reagents. In experiment A5, 625 μl spore suspension (1.25×10⁵ spores) were added to 25 ml of the reagents. Sampling time points were t=0, t=1 h, t=2 h, t=3 h, t=4 h, t=5 h. To differentiate whether CFU originated from vegetative cells or from spores, at each time point samples were split in two equivalent aliquots. One aliquot was subjected to heat treatment (65° C., 30 min) to kill vegetative cells before enumeration of residual heat-resistant spores. The other aliquot was plated at room temperature (RT). Average plate counts (FIGS. 14A-14E and 15A-15E, respectively) are based on counting three plates; error bars indicate +/−one standard deviation.
Experiment A3: At t=45 min recovery of CFU/ml from flasks A and B was reduced to 9% (1.7 CFU/ml) and 43% (8 CFU/ml), respectively, as compared to control flask F. At t=90 min and t=180 min, zero colony forming units (CFU/ml) were recovered from flasks A and B. For flasks C and D retrieval decreased over time but did not drop below 16% (reagent C) and 39% (reagent D) at 180 min. Recovery levels from the flask with reagent E did not decrease (Table 16).

TABLE 16
Experiment A3: CFU/ml % recovery
(as compared to control flask F)

| | 0 min | 10 min | 45 min | 90 min | 180 min |
|---|---|---|---|---|---|
| A | 85 | 81 | 9 | 0 | 0 |
| B | 121 | 66 | 43 | 0 | 0 |
| C | 142 | 77 | 82 | 48 | 16 |
| D | 108 | 81 | 55 | 64 | 39 |
| E | 119 | 65 | 94 | 144 | 95 |
| F | 100 | 100 | 100 | 100 | 100 |

Experiments A4, A5: In both experiments CFU/ml recovery from flasks A and B at t=0 and t=1 h originated from heat-sensitive cells because colony counts were zero for the samples which received heat treatment. No CFU/ml were retrieved from flask A or B for t=2 h, t=3 h, t=4 h (FIGS. 14A-14E and 15A-15E). For both reagents C and D % recovery decreased over time but of all compounds tested reagents A and B killed most effectively (Tables 17-20). Reagent E was not more effective than the water control F (FIGS. 14A-14E and 15A-15E).

TABLE 17
Experiment A4 absent heat: CFU/ml % recovery
(as compared to control flask F): RT

| | 0 min | 1 h | 2 h | 3 h | 4 h |
|---|---|---|---|---|---|
| A | 81 | 2 | 0 | 0 | 0 |
| B | 85 | 12 | 0 | 0 | 0 |
| C | 81 | 71 | 33 | 23 | 15 |
| D | 89 | 54 | 27 | 30 | 15 |
| E | 85 | 90 | 87 | 98 | 79 |
| F | 100 | 100 | 100 | 100 | 100 |

TABLE 18
Experiment A4 with heat: CFU/ml % recovery
(as compared to control flask F): 65° C.

| | 0 min | 1 h | 2 h | 3 h | 4 h |
|---|---|---|---|---|---|
| A | 0 | 0 | 0 | 0 | 0 |
| B | 0 | 0 | 0 | 0 | 0 |
| C | 27 | 13 | 6 | 8 | 0 |
| D | 70 | 78 | 45 | 33 | 46 |
| E | 48 | 53 | 74 | 68 | 114 |
| F | 100 | 100 | 100 | 100 | 100 |

TABLE 19
Experiment A5 absent heat: CFU/ml % recovery
(as compared to control flask F): RT

| | 0 min | 1 h | 2 h | 3 h | 4 h |
|---|---|---|---|---|---|
| A | 128 | 6 | 0 | 0 | 0 |
| B | 124 | 6 | 0 | 0 | 0 |
| C | 97 | 58 | 44 | 32 | 16 |
| D | 105 | 80 | 46 | 67 | 37 |
| E | 122 | 117 | 103 | 113 | 103 |
| F | 100 | 100 | 100 | 100 | 100 |

TABLE 20
Experiment A5 with heat: CFU/ml % recovery
(as compared to control flask F): 65° C.

| | 0 min | 1 h | 2 h | 3 h | 4 h |
|---|---|---|---|---|---|
| A | 0 | 0 | 0 | 0 | 0 |
| B | 0 | 0 | 0 | 0 | 0 |
| C | 58 | 32 | 18 | 8 | 8 |
| D | 75 | 58 | 34 | 34 | 14 |
| E | 71 | 69 | 53 | 71 | 54 |
| F | 100 | 100 | 100 | 100 | 100 |

While reagents C and D in a 4-hour time frame had a negative effect on spore survival, neither one of these reagents was as effective in killing spores as reagents A and B. Reagent E was not different from the water control F.

Viable cell counts demonstrated that reagents A and B affected heat sensitivity of spores very quickly at the t=0 time point suggesting induction of a break in spore dormancy. Chemical disinfectants which are not toxic and able to diminish resistance of spores to killing are potentially of great benefit.

### Example 21

Isolates: Bacillus subtilis strain ATCC #82 and B. cereus ATCC#10987 were obtained from ATCC, and B. circulans #47-10 and #31028 were from collection at Center for Food Safety. The frozen isolates were grown in brain heart infusion agar (BHA) at 37° C. for 24 h. Alicyclobacillus acidocaldarius strain OS-CAJ and SAC (isolated from apple juice concentrate), and N-1108 (isolated from apple-cranberry juice) were from collection at Center for Food Safety. The isolated were grown in Orange Serum Broth at 43° C. for 72 h and then transferred to potato dextrose agar (PDA) at 43° C. for 48 h.
Spore production: For B. cereus, B. subtilis, and B. circulans, the isolates were individually grown in 10-ml BHI for 24 hours and then, precipitated, suspended and washed for 3 times by centrifugation at 4,000×g for 20 min. The final pellet was transferred to 10-ml sporulation medium, containing FeCl₂, 0.0036 mM; MgCl₂, 0.041 mM; MnCl₂, 0.1 mM; NH₄Cl, 10 mM; Na₂SO₄, 0.75 mM; KH₂PO₄, 0.5 mM; CaCl₂, 1 mM; NH₄NO₃, 1.2 mM; D-glucose, 10 mM; and L-glutamic acid, 10 mM, pH 7.1 (Donnellan et al., (1964) J. Bact. 87: 332-335) at 30° C. for 5 days with agitation at 200 RPM. The spores were precipitated and suspended in 1-ml sterile H₂O by centrifugation at 4,000×g for 20 min. The solution was heated at 65° C. for 30 min and kept at 4° C. before use. For A. acidoterrestris isolates, the bacterium was individually grown in potato dextrose agar, pH 3.5 at 43° C. for 7 days and bacteria were collected by a plastic loop, suspended in 5-ml sterile H₂O containing 30 glass beads and vortexed for 2 min at 150 rpm. The solution was heated at 65° C. for 30 min and kept in 5° C. before use.
Spore staining: The Wirtz-Conklin spore stain was used for observation of spore morphology.
Chemicals: Levulinic acid and sodium dodecyl sulfate were obtained from Sigma-Aldrich (St. Louis, Mo.).
Water: Deionized, unchlorinated water was filter sterilized through a 0.2-μm regenerated cellulose filter (Corning Inc., Corning, N.Y.) was used for preparing chemical solution.
Inactivation of spores: Each 500-ml flasks containing 199-ml combined chemical solution with a magnetic bar at 200 rpm was individually heated to 62° C.±2° C. in a hot plate. A volume of 1.0-ml spore was added in the center of the chemical solution under constant mixing condition at 200 rpm.
Enumeration of spores: At pre-determined schedules a sample of 1.0-ml was removed from the flask and mixed with 9.0-ml 0.1 M phosphate buffer, pH 7.2 and then serial dilution (1:10) up to 10⁻⁸ CFU/ml was made and 0.1-ml from each diluted tubes was inoculated on the surface of either BHA plates for bacillus species or PDA plates for alicyclobacillus species. The plates were incubated at 37° C., 48 h for bacillus and at 43° C., 72 h for alicyclobacillus species. The species of colonies randomly picked from the highest dilution plates were confirmed by biochemical assays.

### Example 22

TABLE 21
B. subtilis (strain) ATCC #82 spores treated
by levulinic acid and SDS at 21° C.

| | 0.5% levulinic | 2% levulinic | 3% levulinic | |
|---|---|---|---|---|
| Tim- | acid + | acid + | acid + | H₂O |
| ing | 0.05% SDS | 1% SDS | 2% SDS | only |
| (min) | Counts of spores (log CFU/ml) | | | |
| 0 | <0.7 | <0.7 | <0.7 | 5.5 ± 0.3 |
| 1 | <0.7 | <0.7 | <0.7 | 5.3 ± 0.1 |
| 2 | <0.7 | <0.7 | <0.7 | 5.4 ± 0.1 |
| 5 | <0.7 | <0.7 | <0.7 | 5.4 ± 0.1 |
| 10 | <0.7 | <0.7 | <0.7 | 5.3 ± 0.3 |
| 20 | <0.7 | <0.7 | <0.7 | 5.5 ± 0.1 |
| 30 | <0.7 | <0.7 | <0.7 | 5.3 ± 0.2 |
| 60 | <0.7 | <0.7 | <0.7 | 5.4 ± 0.1 |

^{a} Inoculation of spore is 5.0 × 10⁷ CFU/ml after heating at 65° C. for 30 min.

### Example 23

TABLE 22
B. subtilis (strain ATCC #31028) spores treated
by levulinic acid and SDS at 21° C.

| | | | | |
|---|---|---|---|---|
| | 3% levulinic | | 3% levulinic | |
| Tim- | acid + | | acid + | |
| ing | 2% SDS | H₂O only | 2% SDS | H₂O only |

| | |
|---|---|
| (min) | Counts of spores (log CFU/ml) |

| | | | | |
|---|---|---|---|---|
| 1 | 6.2 ± 0.2 | 7.0 ± 0.1 | 5.8 ± 0.1 | 6.4 ± 0.2 |
| 3 | 6.2 ± 0.1 | ND | 5.9 ± 0.2 | ND |
| 5 | 6.1 ± 0.4 | ND | 5.8 ± 0.2 | ND |
| 10 | 6.1 ± 0.2 | ND | 5.7 ± 0.0 | ND |
| 20 | 6.1 ± 0.3 | ND | 5.8 ± 0.1 | ND |
| 30 | 5.9 ± 0.2 | ND | 5.7 ± 0.2 | ND |
| 60 | 6.2 ± 0.2 | ND | 5.8 ± 0.2 | ND |
| 90 | 6.2 ± 0.1 | ND | 5.8 ± 0.1 | ND |
| 120 | 6.3 ± 0.3 | 7.1 ± 0.3 | 5.8 ± 0.5 | 6.4 ± 0.3 |

^{a} Inoculation of spore is 2.7 × 10⁸ CFU/ml after heating at 65° C. for 30 min.

### Example 24

TABLE 23
B. subtilis (strain ATCC #31028) spores treated
by levulinic acid and SDS at 21° C.

| | | |
|---|---|---|
| Timing | 10% levulinic acid + 2% SDS | H₂O only |

| | |
|---|---|
| (min) | Counts of spores (log CFU/ml) |

| | | |
|---|---|---|
| 5 | 6.2 ± 0.1 | 6.5 ± 0.3 |
| 10 | 5.9 ± 0.2 | ND |
| 30 | 6.0 ± 0.2 | ND |
| 60 | 6.2 ± 0.1 | ND |
| 120 | 6.1 ± 0.3 | ND |
| 180 | 6.2 ± 0.2 | ND |
| 240 | 6.1 ± 0.1 | 6.4 ± 0.2 |

### Example 25

TABLE 24
B. subtilis (strain ATCC #31028) spores treated
by levulinic acid and SDS at 21° C.

| | | |
|---|---|---|
| Timing | 20% levulinic acid + 3% SDS | H₂O only |

| | |
|---|---|
| (min) | Counts of spores (log CFU/ml) |

| | | |
|---|---|---|
| 5 | 6.0 ± 0.1 | 6.6 ± 0.3 |
| 15 | 6.1 ± 0.2 | ND |
| 30 | 5.9 ± 0.2 | ND |
| 60 | 6.0 ± 0.3 | ND |
| 90 | 5.8 ± 0.2 | ND |
| 120 | 5.6 ± 0.2 | 6.2 ± 0.5 |

### Example 26

TABLE 25
B. subtilis(strain ATCC #31028) spores treated
by levulinic acid and SDS at 62° C.

| | | |
|---|---|---|
| Timing | 3% levulinic acid + 2% SDS | H₂O only |

| | |
|---|---|
| (min) | Counts of spores (log CFU/ml) |

| | | |
|---|---|---|
| 1 | <0.7 | ND |
| 5 | <0.7 | ND |
| 15 | <0.7 | ND |
| 30 | <0.7 | ND |
| 60 | <0.7 | 5.0 ± 0.0 |

^{a} Inoculums of B. subtilis ATCC #31028 is 1.6 × 10⁹/ml (germinated and spores).

### Example 27

TABLE 26
B. circulans (strain #47-10) spores treated
by levulinic acid and SDS at 21° C.

| | | |
|---|---|---|
| Timing | 0.5% levulinic acid + 0.05% SDS | H₂O only |

| | |
|---|---|
| (min) | Counts of spores (log CFU/ml) |

| | | |
|---|---|---|
| 0 | 2.2 ± 0.3 | 4.3 ± 0.4 |
| 1 | 2.4 ± 0.1 | ND |
| 2 | 1.9 ± 0.3 | ND |
| 5 | 2.1 ± 0.1 | 4.4 ± 0.1 |
| 10 | 2.1 ± 0.2 | ND |
| 20 | 1.6 ± 0.0 | ND |
| 30 | 1.0 ± 0.0 | 4.6 ± 0.1 |
| 60 | 1.3 ± 0.0 | 4.5 ± 0.2 |

^{a} Inoculation of spore is 8.8 × 10⁶ CFU/ml after heating at 65° C. for 30 min.

### Example 28

TABLE 27
B. circulans (strain #47-10) spores treated
by levulinic acid and SDS at 21° C.

| | | |
|---|---|---|
| Timing | 3% levulinic acid + 2% SDS | H₂O only |

| | |
|---|---|
| (min) | Counts of spores (log CFU/ml) |

| | | |
|---|---|---|
| 1 | 5.2 ± 0.2 | 6.2 ± 0.2 |
| 3 | 5.5 ± 0.1 | ND |
| 5 | 5.0 ± 0.2 | ND |
| 10 | 5.1 ± 0.1 | ND |
| 20 | 5.2 ± 0.2 | ND |
| 30 | 5.2 ± 0.1 | ND |
| 60 | 4.9 ± 0.1 | ND |
| 90 | 4.6 ± 0.1 | ND |
| 120 | 4.4 ± 0.1 | ND |

^{a} Inoculation of spore is 8.2 × 10⁸ CFU/ml after heating at 65° C. for 30 min.

### Example 29

TABLE 28
B. circulans (strain #47-10) spores treated
by levulinic acid and SDS at 21° C.

| | | |
|---|---|---|
| Timing | 3% levulinic acid + 2% SDS | H₂O only |

| | |
|---|---|
| (min) | Counts of spores (log CFU/ml) |

| | | |
|---|---|---|
| 1 | 4.5 ± 0.2 | 5.0 ± 0.2 |
| 3 | 4.5 ± 0.3 | ND |
| 5 | 4.6 ± 0.2 | ND |
| 10 | 4.6 ± 0.1 | ND |
| 20 | 4.5 ± 0.1 | ND |
| 30 | 4.6 ± 0.0 | ND |
| 60 | 4.5 ± 0.0 | ND |
| 90 | 4.4 ± 0.0 | ND |
| 120 | 4.0 ± 0.1 | 5.0 ± 0.2 |

^{a} Inoculation of spore is 9.8 × 10⁶ CFU/ml after heating at 65° C. for 30 min.

### Example 30

TABLE 29
B. circulans (strain #47-10) spores treated
by levulinic acid and SDS at 21° C.

| | | |
|---|---|---|
| Timing | 10% levulinic acid + 2% SDS | H₂O only |

| | |
|---|---|
| (min) | Counts of spores (log CFU/ml) |

| | | |
|---|---|---|
| 5 | 5.0 ± 0.1 | 5.0 ± 0.2 |
| 10 | 4.9 ± 0.1 | ND |
| 30 | 4.3 ± 0.2 | ND |
| 60 | 3.5 ± 0.0 | ND |
| 120 | 2.8 ± 0.1 | ND |
| 180 | <0.7 | ND |
| 240 | <0.7 | 5.0 ± 0.2 |

^{a} Inoculation of spore is 9.3 × 10⁶ CFU/ml after heating at 65° C. for 30 min.

### Example 31

TABLE 30
B. cereus (strain ATCC#10987) spores treated
by levulinic acid and SDS at 21° C.

| | | |
|---|---|---|
| Timing | 0.5% levulinic acid + 0.05% SDS | H₂O only |

| | |
|---|---|
| (min) | Counts of spores (log CFU/ml) |

| | | |
|---|---|---|
| 0 | 4.8 ± 0.1 | 4.8 ± 0.1 |
| 1 | 4.8 ± 0.3 | ND |
| 2 | 4.7 ± 0.1 | ND |
| 5 | 4.4 ± 0.3 | 4.8 ± 0.2 |
| 10 | 4.2 ± 0.4 | ND |
| 20 | 3.8 ± 0.2 | ND |
| 30 | 3.8 ± 0.1 | 4.9 ± 0.1 |
| 60 | 3.7 ± 0.1 | 4.8 ± 0.2 |

^{a} Inoculation of spore is 2.2 × 10⁷ CFU/ml after heating at 65° C. for 30 min.

### Example 32

TABLE 31
B. cereus (strain ATCC#10987) spores treated
by levulinic acid and SDS at 21° C.

| | | |
|---|---|---|
| Timing | 3% levulinic acid + 2% SDS | H₂O only |

| | |
|---|---|
| (min) | Counts of spores (log CFU/ml) |

| | | |
|---|---|---|
| 1 | 6.5 ± 0.1 | 6.7 ± 0.1 |
| 3 | 6.5 ± 0.2 | ND |
| 5 | 6.7 ± 0.1 | ND |
| 10 | 6.5 ± 0.1 | ND |
| 20 | 6.4 ± 0.1 | ND |
| 30 | 6.7 ± 0.2 | ND |
| 60 | 6.7 ± 0.1 | ND |
| 90 | 6.7 ± 0.1 | ND |
| 120 | 6.7 ± 0.1 | 7.1 ± 0.2 |

^{a} Inoculation of spore is 4.0 × 10⁸ CFU/ml after heating at 65° C. for 30 min.

### Example 33

TABLE 32
B. cereus (strain ATCC#10987) spores treated
by levulinic acid and SDS at 21° C.

| | 10% levulinic | H₂O | 20% levulinic | H₂O |
|---|---|---|---|---|
| Timing | acid + 2% SDS | only | acid + 3% SDS | only |
| (min) | Counts of spores (log CFU/ml) | | | |
| 5 | 6.2 ± 0.4 | 6.6 ± 0.1 | 6.3 ± 0.3 | 6.7 ± 0.2 |
| 10 | 6.6 ± 0.1 | ND | ND | ND |
| 15 | ND | ND | 6.3 ± 0.2 | ND |
| 30 | 6.1 ± 0.2 | ND | 6.3 ± 0.1 | ND |
| 60 | 6.3 ± 0.3 | ND | 6.3 ± 0.2 | ND |
| 90 | ND | ND | 6.2 ± 0.2 | ND |
| 120 | 6.3 ± 0.0 | ND | 6.0 ± 0.1 | 6.5 ± 0.3 |
| 180 | 6.2 ± 0.1 | ND | 5.8 ± 0.1 | ND |
| 240 | 6.6 ± 0.0 | 6.7 ± 0.2 | ND | ND |

^{a} Inoculation of spore is 6.1 × 10⁸ CFU/ml after heating at 65° C. for 30 min.

### Example 34

TABLE 33
B. cereus spores treated by levulinic acid and
SDS at 62° C. ± 2° C.

| | | 3% levulinic acid + 2% SDS | H₂O only |
|---|---|---|---|
| | Timing | at 62° C. | at 62° C. |
| Strain | (min) | Counts of spores (log CFU/ml) | |
| B. cereus | 0 | <0.7 | 6.1 ± 0.1 |
| (ATCC#10987) | 1 | <0.7 | ND |
| | 5 | <0.7 | ND |
| | 20 | <0.7 | ND |
| | 30 | <0.7 | ND |
| | 60 | <0.7 | 5.7 ± 0.1 |
| B. circulans | 0 | <0.7 | 4.5 ± 0.3 |
| (#47-10) | 1 | <0.7 | ND |
| | 2 | <0.7 | ND |
| | 5 | <0.7 | ND |
| | 10 | <0.7 | ND |
| | 20 | <0.7 | 3.7 ± 0.1 |

### Example 35

TABLE 34
Alicyclobacillus acidoterrestris mixture (bacteria + spores) treated
by levulinic acid and SDS at 62° C. ± 2° C.

| | | 3% levulinic acid + 2% SDS | H₂O at |
|---|---|---|---|
| | Timing | at 62° C. | 62° C. |
| Strain | (min) | Counts of spores (log CFU/ml) | |
| #SAC, #OS- | 0 | <0.7 | 6.5 ± 0.1 |
| CAS, and #N- | 10 | <0.7 | 6.1 ± 0.1 |
| 1108 | 30 | <0.7 | 4.6 ± 0.2 |
| | 60 | <0.7 | 3.8 ± 0.1 |

^{a} Inoculation of a mixture of 3-strains A. acidoterrestris, including strains #SAC, #OS-CAS, and #N-1108 is 1.1 × 10⁹/ml.

### Example 36

TABLE 35
Alicyclobacillus acidoterrestris mixture (bacteria + spores) treated
by levulinic acid and SDS at 21° C. and 62° C.

| | | 3% levulinic | | 3% levulinic | |
|---|---|---|---|---|---|
| | Tim- | acid + 2% | H₂O at | acid + 2% | H₂O at |
| | ing | SDS at 21° C. | 21° C. | SDS at 62° C. | 62° C. |
| Strain | (min) | Counts of spores (log CFU/ml) | | | |
| SAC | 0 | 4.7 ± 0.2 | 4.8 ± 0.1 | ND | ND |
| | 1 | ND | ND | <0.7 | 5.0 ± 0.2 |
| | 5 | ND | ND | <0.7 | 5.1 ± 0.2 |
| | 10 | ND | ND | <0.7 | 5.1 ± 0.1 |
| | 30 | 4.6 ± 0.2 | ND | ND | ND |
| | 60 | 4.7 ± 0.2 | 4.8 ± 0.1 | ND | ND |
| OS- | 0 | 4.5 ± 0.1 | 4.9 ± 0.1 | ND | ND |
| CAS | 1 | ND | ND | <0.7 | 5.0 ± 0.1 |
| | 5 | ND | ND | <0.7 | 5.0 ± 0.3 |
| | 10 | ND | ND | <0.7 | 5.2 ± 0.2 |
| | 30 | 4.4 ± 0.1 | ND | ND | ND |
| | 60 | 4.1 ± 0.2 | 4.9 ± 0.1 | ND | ND |
| N- | 0 | 4.8 ± 0.1 | 5.1 ± 0.2 | ND | ND |
| 1108 | 1 | ND | ND | <0.7 | 5.0 ± 0.1 |
| | 5 | ND | ND | <0.7 | 5.1 ± 0.1 |
| | 10 | ND | ND | <0.7 | 5.1 ± 0.0 |
| | 30 | 4.8 ± 0.1 | ND | ND | ND |
| | 60 | 3.9 ± 0.2 | 5.1 ± 0.1 | ND | ND |

^{a} Inoculation of mixture for isolate SAC is 1.6 × 10⁸ CFU/ml, for OS-CAS is 2.6 × 10⁷ CFU/ml, and for N-1108 is 6.3 × 10⁷ CFU/ml.

### Example 37

TABLE 36
Counts of Alicyclobacillus acidoterrestris
spores (pre-treated for 30 min at 65° C.)

Counts of A. acidoterrestris
(log CFU/ml)

| Bacterial | Timing | 3% levulinic acid + | |
|---|---|---|---|
| isolates | (Sec) | 2% SDS at 62° C. | H₂O at 62° C. |
| SAC | 0 | 4.3 | |
| | 15 | 4.3 | |
| | 30 | 3.4 | |
| | 60 | 3.3 | |
| | 90 | 2.7 | |
| | 300 | | 5.2 |
| OS-CAS | 0 | 4.3 | |
| | 15 | 4.3 | |
| | 30 | 4.4 | |
| | 60 | 4.0 | |
| | 90 | 3.4 | |
| | 300 | | 5.4 |
| N-1108 | 0 | 4.2 | |
| | 15 | 4.5 | |
| | 30 | 4.4 | |
| | 60 | 4.2 | |
| | 90 | 3.7 | |
| | 300 | | 5.3 |

^{a} Inoculation of spore (after treated at 65° C. for 30 min) for isolate SAC is 8.0 × 10⁷ CFU/ml, for OS-CAS is 7.2 × 10⁷ CFU/ml, and for N-1108 is 6.3 × 10⁷ CFU/ml.

TABLE 37
Effect of levulinic acids plus SDS at 21° C. on various yeast species

| | Yeast counts (log CFU/ml) at min: | | | | | | | |
|---|---|---|---|---|---|---|---|---|
| Yeast Name^{a} | 0^{b} | 1 | 2 | 5 | 10 | 20 | 30 | 60 |
| Saccharomyces cerevisiae in 0.1M PBS | 5.2 | 5.3 | 5.5 | 5.3 | 5.3 | 5.2 | 5.3 | 5.3 |
| (Control) | | | | | | | | |
| Saccharomyces cerevisiae in 2.0% | 5.4 | 5.3 | 5.5 | 5.4 | 5.3 | 5.3 | 5.5 | 5.0 |
| levulinic acid (Control) | | | | | | | | |
| S. cerevisiae in 1.0% SDS (Control) | 2.7 | 2.4 | 2.6 | 2.3 | 2.8 | 2.7 | 2.3 | 2.4 |
| S. cerevisiae in 0.5% levulinic + 0.05% | 4.9 | 4.5 | 3.9 | 3.2 | 2.7 | 1.7 | 1.3 | <0.7 |
| SDS | | | | | | | | |
| S. cerevisiae in 2% levulinic acid + 1.0% | 0.7 | — | — | — | — | — | — | — |
| SDS | | | | | | | | |
| Debaryomyces hansenii in 0.1M PBS | 4.8 | 4.9 | 4.9 | 4.8 | 4.8 | 4.8 | 4.8 | 4.8 |
| (Control) | | | | | | | | |
| D. hansenii in 2.0% levulinic acid (Control) | 4.9 | 4.8 | 4.9 | 4.6 | 4.4 | 4.7 | 3.0 | 1.3 |
| D. hansenii in 1.0% SDS (Control) | 4.5 | 4.5 | 4.1 | 4.5 | 4.4 | 4.5 | 4.5 | 4.4 |
| D. hansenii in 0.5% levulinic acid + 0.05% | 4.9 | 4.9 | 4.7 | 4.5 | 4.3 | 3.7 | 3.1 | 1.7 |
| SDS | | | | | | | | |
| D. hansenii 2% levulinic acid + 1% SDS | 1.0 | — | — | — | — | — | — | — |
| Candida magnoliae in 0.1M PBS (Control) | 5.9 | 5.8 | 6.1 | 5.9 | 5.9 | 5.9 | 5.9 | 5.7 |
| C. magnoliae in 2.0% levulinic acids | 6.0 | 5.9 | 5.9 | 5.9 | 6.0 | 6.0 | 5.9 | 5.8 |
| (Control) | | | | | | | | |
| C. magnoliae in 1.0% SDS (Control) | 3.5 | 3.5 | 3.3 | 3.2 | 3.2 | 2.7 | 3.0 | 3.1 |
| C. magnoliae in 0.5% levulinic acids + | 4.0 | 3.6 | 3.2 | 2.1 | 1.3 | <0.7 | <0.7 | <0.7 |
| 0.05% SDS | | | | | | | | |
| C. magnoliae in 2.0% levulinic acid + 1.0% | 2.1 | 0.7 | — | — | — | — | — | — |
| SDS | | | | | | | | |
| Zygosaccharomyces bailii in 0.1M PBS | 5.4 | 5.5 | 5.6 | 5.5 | 5.3 | 5.4 | 5.6 | 5.5 |
| (Control) | | | | | | | | |
| Z. bailii in 2% levulinic acid (Control) | 5.4 | 5.4 | 5.5 | 5.4 | 5.4 | 5.4 | 5.4 | 5.3 |
| Z. bailii in 1.0% SDS (Control) | 4.6 | 4.7 | 4.6 | 4.6 | 4.6 | 4.5 | 4.5 | 4.4 |
| Z. bailii in 0.5% levulinic acid + 0.05% SDS | 5.0 | 5.0 | 4.8 | 3.6 | 3.8 | 2.3 | 2.6 | <0.7 |
| Z. bailii in 2% levulinic acid + 1% SDS | 4.6 | 4.2 | 3.9 | 2.9 | 2.0 | <0.7 | <0.7 | <0.7 |
| Geotrichum candidum 0.1M PBS (Control) | 4.6 | 4.7 | 4.8 | 4.7 | 4.7 | 4.5 | 4.6 | 4.6 |
| G. candidum in 2.0% levulinic acid | 4.6 | 4.4 | 4.4 | 4.3 | 4.1 | 3.8 | 3.4 | 2.0 |
| (Control) | | | | | | | | |
| G. candidum in 1.0% SDS (Control) | 3.6 | 3.8 | 3.3 | 3.5 | 3.7 | 3.5 | 3.4 | 3.3 |
| G. candidum in 0.5% levulinic acid + | 3.0 | 2.6 | 2.6 | 2.4 | <0.7 | <0.7 | <0.7 | <0.7 |
| 0.05% SDS | | | | | | | | |
| G. candidum in 2.0% levulinic acid + 1.0% | 3.3 | <0.7 | — | — | — | — | — | — |
| SDS | | | | | | | | |

^{a}Initial inoculation level: Saccharomyces cerevisiae: 7.5 × 10⁷ CFU/ml; Debaryomyces hansenii: 7.4 × 10⁷ CFU/ml; Candida magnoliae: 3.4 × 10⁸ CFU/ml; Zygosaccharomyces bailii: 3.4 × 10⁷ CFU/ml; Geotrichum candidum: 1.2 × 10⁷ CFU/ml.
^{b}The actual time 0 was delayed by 5 to 10 seconds due to time for sample processing.
^{c}Negative by direct plating and enrichment culture.

TABLE 38
Effect of levulinic acids plus SDS at 21° C. on various mold species

| | Mold counts (log CFU/ml) at min: | | | | | | | |
|---|---|---|---|---|---|---|---|---|
| Mold Name^{a} | 0^{b} | 1 | 2 | 5 | 10 | 20 | 30 | 60 |
| Mucor hiemalis in 0.1M PBS (Control) | 6.1 | 6.1 | 6.1 | 6.2 | 6.4 | 6.1 | 6.1 | 6.2 |
| M. hiemalis in 3.0% levulinic acids | 6.1 | 6.2 | 6.2 | 6.0 | 5.8 | 5.1 | 4.8 | 4.1 |
| (Control) | | | | | | | | |
| M. hiemalis in 2.0% SDS (Control) | 6.0 | 5.8 | 5.8 | 6.0 | 5.9 | 5.8 | 5.8 | 5.4 |
| M. hiemalis in 0.5% levulinic + 0.05% SDS | 5.8 | 5.9 | 6.0 | 6.1 | 5.5 | 5.8 | 5.9 | 5.4 |
| M. hiemalis in 2% levulinic acid + 1.0% | 5.1 | 5.0 | 4.9 | 4.9 | 4.5 | 3.7 | 3.4 | 2.5 |
| SDS | | | | | | | | |
| M. hiemalis in 3% levulinic acid + 2% SDS | 5.6 | 5.6 | 5.5 | 5.0 | 4.7 | 4.6 | 3.2 | 2.4 |
| Penicillium pubeseus in 0.1M PBS | 4.9 | 4.8 | 4.8 | 4.8 | 4.9 | 4.8 | 4.9 | 4.7 |
| (Control) | | | | | | | | |
| P. pubeseus in 3.0% levulinic acid | 5.2 | 4.9 | 4.8 | 5.2 | 4.0 | 3.2 | 2.7 | 1.7 |
| P. pubeseus in 2.0% SDS | 4.4 | 4.3 | 4.4 | 4.4 | 4.5 | 4.5 | 4.4 | 4.4 |
| P. pubeseus in 0.5% levulinic acid + | 5.1 | 5.1 | 5.1 | 5.0 | 5.0 | 4.9 | 4.9 | 4.5 |
| 0.05% SDS | | | | | | | | |
| P. pubeseus 2% levulinic acid + 1% SDS | 5.2 | 5.2 | 5.1 | 5.0 | 4.8 | 4.6 | 4.4 | 4.2 |
| P. pubeseus in 3% levulinic acid + 2% | 3.5 | 3.2 | <0.7 | <0.7 | <0.7 | <0.7 | <0.7 | <0.7 |
| SDS | | | | | | | | |
| Penicillium. expansum in 0.1M PBS | 4.4 | 4.4 | 4.5 | 4.8 | 4.6 | 4.0 | 4.5 | 4.5 |
| (Control) | | | | | | | | |
| P. expansum in 3.0% levulinic acids | 4.3 | 4.4 | 4.2 | 4.0 | 3.7 | 3.3 | 3.3 | 2.4 |
| P. expansum in 2.0% SDS | 4.2 | 4.1 | 3.8 | 3.4 | 3.5 | 3.5 | 3.5 | 3.6 |
| P. expansum in 0.5% levulinic acid + | 4.5 | 3.9 | 3.6 | 3.2 | 2.8 | 2.7 | 2.0 | 2.0 |
| 0.05% SDS | | | | | | | | |
| P. expansum in 2.0% levulinic acids + | 4.1 | 3.7 | 3.6 | 3.4 | 3.3 | 3.0 | 2.5 | 1.7 |
| 1.0% SDS | | | | | | | | |
| Paecylomyces expansum in 3% levulinic | 3.9 | <0.7 | <0.7 | <0.7 | <0.7 | <0.7 | <0.7 | <0.7 |
| acid + 2% SDS | | | | | | | | |
| P. variotri in 0.1M PBS (Control) | 5.5 | 5.5 | 5.7 | 5.4 | 5.5 | 5.5 | 5.5 | 5.5 |
| P. variotri in 3.0% levulinic acid | 5.6 | 5.6 | 5.4 | 5.6 | 5.6 | 5.3 | 5.4 | 4.6 |
| P. variotri in 2.0% SDS | 5.6 | 5.5 | 5.5 | 5.4 | 5.4 | 5.6 | 5.6 | 5.6 |
| P. variotri in 0.5% levulinic acid + 0.05% | 5.2 | 5.2 | 4.8 | 4.5 | 4.1 | 4.4 | 3.8 | 3.4 |
| SDS | | | | | | | | |
| P. variotri in 2.0% levulinic acid + 1.0% | 4.3 | 3.8 | 3.4 | 3.0 | 2.7 | 2.3 | 1.5 | 0.7 |
| SDS | | | | | | | | |
| P. variotri in 3.0% levulinic acid + 2.0% | 4.6 | 4.4 | 4.4 | 3.6 | 2.9 | 2.2 | <0.7 | <0.7 |
| SDS | | | | | | | | |

^{a}Initial inoculation level: Mucor hiemalis: 3.1 × 10⁷ CFU/ml; Penicillium pubeseus: 6.9 × 10⁷ CFU/ml; Penicillium expansum: 2.9 × 10⁷ CFU/ml; Paecylomyces variotri: 2.7 × 10⁷ CFU/ml.
^{b}The actual time 0 was delayed by 5 to 10 seconds due to time for sample processing.

TABLE 39
Inactivation of Alicyclobacillus acidoterrestris spores by 3%
levulinic acid plus 2% SDS at 70° C. or 80° C.

| | | A. acidoterrestris counts (log CFU/ml) | | | |
|---|---|---|---|---|---|
| | | 3% levulinic | | 3% levulinic | |
| | Time | acid + 2% | H₂O at | acid + 2% | H₂O at |
| Strain | (min) | SDS at 70° C. | 70° C. | SDS at 80° C. | 80° C. |
| SAC | 0 | 5.0 | ND | 2.2 | ND |
| | 1 | 3.0 | ND | 0.7 | ND |
| | 2 | <0.7 | ND | <0.7 | ND |
| | 3 | <0.7 | ND | <0.7 | ND |
| | 5 | <0.7 | ND | <0.7 | ND |
| | 10 | <0.7 | 6.0 | <0.7 | 6.0 |
| | 20 | <0.7 | 6.1 | <0.7 | 6.1 |
| OS-CAS | 0 | 5.3 | ND | <0.7 | ND |
| | 1 | 3.0 | ND | <0.7 | ND |
| | 2 | <0.7 | ND | <0.7 | ND |
| | 3 | <0.7 | ND | <0.7 | ND |
| | 5 | <0.7 | ND | <0.7 | ND |
| | 10 | <0.7 | 6.0 | <0.7 | 6.0 |
| | 20 | <0.7 | 6.1 | <0.7 | 5.9 |
| N-1108 | 0 | 4.6 | ND | 3.6 | 5.6 |
| | 1 | 3.7 | ND | <0.7 | ND |
| | 2 | <0.7 | ND | <0.7 | ND |
| | 3 | <0.7 | ND | <0.7 | ND |
| | 5 | <0.7 | ND | <0.7 | ND |
| | 10 | <0.7 | 6.2 | <0.7 | 5.7 |
| | 20 | <0.7 | 6.2 | <0.7 | 5.8 |

^{a} Spore inoculum (after treatment of 65° C. for 30 min) for strain SAC was 9.4 × 10⁷ CFU/ml, for OS-CAS was 1.1 × 10⁸ CFU/ml, and for N-1108 was 1.6 × 10⁸ CFU/ml.

### Example 38

The average S. typhimurium count of apples treated with water only for 1, 2, and 5 min was 2.65, 2.7, and 2.65 log CFU/apple, respectively. The average S. typhimurium count of apples treated with 0.5% levulinic acid plus 0.05% SDS was <0.7, 1.35, and <0.7 log CFU/apple, respectively. The reduction of S. typhimurium on the surface of apples treated with 0.5% levulinic acid plus 0.05% SDS for 1, 2, and 5 min was 2.0, 1.4, and 2.0 log CFU/apple (Table 40). Similar results were obtained for aerobic plate counts (APC). Substantial reduction of yeasts and molds (>1.0 log CFU/apple) on apples required 5 min of exposure.

Following treatment, the microbial counts of the treatment solution containing 0.5% levulinic acid plus 0.05% SDS) were <0.7 log S. typhimurium/ml, and 1.7 log Y&M/ml; containing 50 ppm acidified sodium chlorite were <0.7 log S. typhimurium/ml, and 1.6 log Y&M/ml; with water only were 2.7 log S. typhimurium/ml, and 1.6 log Y&M/ml. The S. typhimurium counts on apples treated for 1, 2, and 5 min with 50 ppm acidified sodium chlorite was 3.25, 3.1, and 2.8 log CFU/apple, respectively, with no reduction of Salmonella counts (Table 40).

TABLE 40
Microbial counts on apples treated at 21° C. for different times in a 4-L tank.

| | | Treated with 0.5% levulinic | | | Treated with 50 ppm acidified | | | | | |
|---|---|---|---|---|---|---|---|---|---|---|
| Apple | | acid plus 0.05% SDS, pH 3.1 | | | sodium chlorite, pH 4.6 | | | Treated with water only | | |
| group | Time | Microbial counts (log CFU/whole apple) | | | | | | | | |
| No. | (min) | Salmonella | APC | Y&M | Salmonella | APC | Y&M | Salmonella | APC | Y&M |
| 1 | 1 | 0.7 | <1.7 | 3.1 | 3.3 | 3.7 | 3.2 | 2.7 | 3.6 | 3.4 |
| | 2 | 1.3 | 2.7 | 3.9 | 3.4 | 3.7 | 3.5 | 2.7 | 3.6 | 2.8 |
| | 5 | <0.7 | <1.7 | 2.3 | 3.2 | 4.9 | 3.9 | 3.0 | 5.0 | 4.0 |
| 2 | 1 | <0.7 | 2.7 | 3.2 | 3.2 | 3.4 | 3.2 | 2.5 | 3.1 | 2.8 |
| | 2 | 1.3 | 1.4 | 1.0 | 2.6 | 2.7 | 3.6 | 2.8 | 3.0 | 2.8 |
| | 5 | 1.2 | <1.7 | 1.3 | 2.9 | 3.2 | 3.7 | 2.8 | 3.0 | 3.0 |
| 3 | 1 | <0.7 | <1.7 | 3.5 | 3.3 | 3.6 | 2.9 | 2.7 | 2.3 | 3.0 |
| | 2 | 1.5 | 2.5 | 3.9 | 3.3 | 3.4 | 3.9 | 2.7 | 3.1 | 2.9 |
| | 5 | <0.7 | <1.7 | 2.4 | 2.4 | 3.9 | 3.2 | 2.5 | 2.9 | 2.9 |
| 4 | 1 | <0.7 | <1.7 | 3.1 | 3.2 | 3.6 | 3.0 | 2.7 | 2.8 | 3.0 |
| | 2 | 1.3 | <1.7 | 3.4 | 3.1 | 4.5 | 3.5 | 2.6 | 2.9 | 3.0 |
| | 5 | <0.7 | 2.3 | 2.1 | 2.7 | 2.9 | 3.3 | 2.3 | 3.4 | 3.2 |

Inoculum level for S. typhimurium was 1.1 × 10⁶ CFU/ml; initial yeast and mold (Y&M) count was 4.0 × 10⁴ CFU/ml.
Background aerobic plant count before inoculation of apple 1 was 4.0 log CFU/apple; of apple 2 was 3.6 log CFU/apple.
Following inoculation, S. typhimurium count of apple 1 was 4.0 log S. typhimurium/apple; apple 2 was 4.8 log S. typhimurium/apple.

### Example 39

The average S. typhimurium count on celery treated with water only for 1, 2, and 5 min was 3.65, 3.57, and 3.5 log CFU/celery, respectively; and on celery treated with 0.5% levulinic acid plus 0.05% SDS was 1.1, 1.0, and 1.3 log CFU/celery, respectively, representing a 2.2-2.6 log CFU S. typhimurium CFU/celery reduction (Table 41). S. typhimurium counts on celery treated with 50 ppm of acidified sodium chlorite for 1, 2, and 5 min were 3.4, 3.1, and 3.0 log CFU, respectively; with a reduction of about 0.5 log S. typhimurium/celery (Table 41). Following treatment, the microbial counts in the treatment solutions were <log 0.7 log S. typhimurium/ml, and 1.3 log Y&M/ml in the 0.5% levulinic acid plus 0.05% SDS-treatment solution; and were <0.7 log S. typhimurium and 2.3 log Y&M/ml in the 50 ppm acidified sodium chlorite solution; and were 3.2 log S. typhimurium/ml, and 3.5 log Y&M/ml in the water-treatment solution.

TABLE 41
Microbial counts on celery treated with different chemicals at 21° C. for different times in a 4-L tank.

| | | Treated with 0.5% levulinic | | | Treated with 50 ppm acidified | | | | | |
|---|---|---|---|---|---|---|---|---|---|---|
| Celery | | acid plus 0.05% SDS, pH 3.1 | | | sodium chlorite, pH 4.6 | | | Treated with water only | | |
| group | Time | Microbial counts (log CFU/celery) | | | | | | | | |
| No. | (min) | Salmonella | APC | Y&M | Salmonella | APC | Y&M | Salmonella | APC | Y&M |
| 1 | 1 | 2.0 | 4.5 | 3.4 | 3.6 | 6.4 | 5.5 | 3.8 | 6.0 | 5.5 |
| | 2 | 0.7 | 4.9 | 2.3 | 3.1 | 5.9 | 5.1 | 3.8 | 6.3 | 5.5 |
| | 5 | 1.5 | 3.3 | 3.4 | 2.8 | 4.5 | 4.9 | 3.7 | 6.0 | 4.9 |
| 2 | 1 | 0.7 | 3.1 | 4.1 | 3.3 | 5.4 | 5.2 | 4.0 | 5.8 | 4.4 |
| | 2 | 1.4 | 2.8 | 4.1 | 3.1 | 4.6 | 5.0 | 3.6 | 6.2 | 5.5 |
| | 5 | 1.4 | 2.8 | 4.1 | 2.8 | 5.4 | 5.0 | 3.3 | 5.1 | 5.2 |
| 3 | 1 | 1.0 | 2.7 | 3.9 | 3.3 | 6.1 | 5.5 | 3.1 | 5.3 | 5.4 |
| | 2 | 0.7 | 3.0 | 3.9 | 3.1 | 5.9 | 5.4 | 3.2 | 4.8 | 5.1 |
| | 5 | 1.4 | 5.3 | 4.6 | 2.9 | 3.7 | 5.1 | 3.4 | 5.9 | 5.3 |
| 4 | 1 | 0.7 | 3.5 | 4.5 | 3.4 | 6.0 | 5.5 | 3.7 | 5.6 | 4.9 |
| | 2 | 1.2 | 4.7 | 3.4 | 3.2 | 5.2 | 5.2 | 3.7 | 5.1 | 4.8 |
| | 5 | 1.0 | 3.6 | 3.4 | 3.6 | 6.2 | 5.4 | 3.6 | 5.1 | 5.2 |

Inoculum level for S. typhimurium was 1.2 × 10⁶ CFU/ml; initial yeast and mold (Y&M) count was 1.0 × 10⁵ CFU/ml.
Background aerobic plate count before inoculation of celery 1 was 7.0 log CFU/celery; of celery 2 was 7.0 log CFU/celery.
Following inoculation, S. typhimurium count of celery 1 was 5.2 log S. typhimurium/celery; celery 2 was 4.8 log S. typhimurium/celery.

### Example 40

The average S. typhimurium counts on onions treated with water only for 1, 2, and 5 min at 21° C. were 4.2, 4.0, and 4.0 log CFU/onion, respectively; whereas the average S. Typhimurium counts on onions treated with 0.5% levulinic acid plus 0.05% SDS for 1, 2, and 5 min were 2.07, 2.05, and 1.65 CFU/onion, respectively, representing an average reduction of 2.13, 1.95, and 2.3 log S. typhimurium CFU/onion, respectively (Table 42). Treatment with 50 ppm acidified sodium chlorite resulted in a small reduction (<0.5 log CFU/onion) of S. typhimurium, APC, and yeast and mold counts (Table 42).

Following treatment, the microbial counts of the treatment solutions revealed the counts were <log 0.7 log S. typhimurium/ml and 2.7 log Y&M/ml in the 0.5% levulinic acid plus 0.05% SDS-treatment solution; were <0.7 log S. typhimurium, and 2.6 log Y&M/ml in the 50 ppm acidified sodium chlorite treatment solution; and were 3.2 log S. typhimurium/ml and 3.1 log Y&M/ml in the water-treatment solution.

TABLE 42
Microbial counts on onions treated at 21° C. for different times in a 4-L tank.

| | | Treated with 0.5% levulinic | | | Treated with 50 ppm acidified | | | | | |
|---|---|---|---|---|---|---|---|---|---|---|
| Onion | | acid plus 0.05% SDS, pH 3.1 | | | sodium chlorite, pH 4.6 | | | Treated with water only | | |
| group | Time | Microbial counts (log CFU/whole onion) | | | | | | | | |
| No. | (min) | Salmonella | APC | Y&M | Salmonella | APC | Y&M | Salmonella | APC | Y&M |
| 1 | 1 | 2.4 | 4.9 | 3.1 | 4.1 | 5.2 | 4.6 | 4.2 | 5.5 | 3.9 |
| | 2 | 2.2 | 3.6 | 4.7 | 4.0 | 6.0 | 4.7 | 4.2 | 5.5 | 4.7 |
| | 5 | 1.7 | 4.4 | 2.9 | 3.5 | 6.4 | 4.5 | 3.8 | 6.4 | 5.4 |
| 2 | 1 | 2.0 | 4.4 | 2.9 | 4.0 | 4.4 | 3.8 | 4.2 | 6.3 | 4.8 |
| | 2 | 1.9 | 4.1 | 4.9 | 3.9 | 6.3 | 3.8 | 4.0 | 5.2 | 3.7 |
| | 5 | 1.8 | 3.8 | 4.7 | 4.0 | 6.4 | 4.3 | 3.9 | 5.7 | 4.3 |
| 3 | 1 | 1.9 | 5.2 | 4.0 | 3.8 | 5.3 | 3.8 | 4.1 | 6.1 | 4.2 |
| | 2 | 2.1 | 3.1 | 4.1 | 3.8 | 6.7 | 3.4 | 3.7 | 4.7 | 4.5 |
| | 5 | 2.0 | 3.4 | 3.9 | 3.6 | 5.9 | 3.3 | 3.8 | 5.9 | 3.6 |
| 4 | 1 | 2.0 | 4.5 | 3.4 | 4.0 | 5.3 | 4.6 | 4.3 | 6.4 | 4.6 |
| | 2 | 1.0 | 3.9 | 4.2 | 3.9 | 5.4 | 4.6 | 4.2 | 5.7 | 3.3 |
| | 5 | 1.1 | 3.9 | 5.4 | 3.6 | 5.2 | 4.0 | 4.3 | 6.2 | 4.1 |

Inoculum level for S. typhimurium was 1.0 × 10⁶ CFU/ml.
Background aerobic plate count before inoculation of onion 1 was 6.4 log CFU/onion; of onion 2 was 5.2 log CFU/onion.
Following inoculation, S. typhimurium count of onion 1 was 5.1 log S. typhimurium per onion; onion 2 was 5.2 log S. typhimurium per onion.

### Example 41

Most cantaloupes contain dirt at different degrees thereby increasing the challenge for killing microbes by chemical wash treatments. The average S. typhimurium count, aerobic plate count, and yeast and mold count on cantaloupes treated by water only for 5 min at 21° C. were 3.76, 5.07, and 4.94 log CFU per cantaloupe, respectively. The average S. typhimurium count, aerobic plate count, and yeast and mold on cantaloupes treated with 1.0% levulinic acid plus 0.1% SDS for 5 min were 1.5, 4.2, and 4.46 log CFU per cantaloupe, respectively (Table 32), hence, the average reduction of S. typhimurium, APC, and yeast and mold counts were 2.26, 0.87, and 0.48 log CFU per cantaloupe, respectively. The S. typhimurium counts on cantaloupes treated with 50 ppm acidified sodium chlorite were reduced by only 0.46 log CFU per cantaloupe (Table 43).

Following treatment, the microbial counts in the treatment solutions were <0.7 log S. typhimurium/ml, and 1.7 log Y&M/ml for the 1.0% levulinic acid plus 0.1% SDS, <0.7 log S. typhimurium, and 3.9 log Y&M/ml for 50 ppm acidified sodium chlorite, and 3.9 log S. typhimurium/ml, and 1.8 log Y&M/ml for water.

TABLE 43
Microbial counts on cantaloupes at 21° C. for 5 minutes in a 4-L tank.

| | | Treated with 1.0% levulinic | | | Treated with 50 ppm acidified | | | | | |
|---|---|---|---|---|---|---|---|---|---|---|
| Canta- | | acid plus 0.1% SDS, pH 3.1 | | | sodium chlorite, pH 4.6 | | | Treated with water only | | |
| loupe | Time | Microbial counts (log CFU/whole cantaloupe) | | | | | | | | |
| No. | (min) | Salmonella | APC | Y&M | Salmonella | APC | Y&M | Salmonella | APC | Y&M |
| 1 | 5 | 1.4 | 5.2 | 4.6 | 3.0 | 4.9 | 4.8 | 3.5 | 5.4 | 5.4 |
| 2 | 5 | 1.3 | 3.8 | 4.0 | 3.0 | 5.0 | 5.3 | 3.8 | 5.3 | 4.9 |
| 3 | 5 | 1.3 | 4.2 | 3.8 | 2.8 | 5.3 | 5.4 | 3.6 | 4.6 | 4.7 |
| 4 | 5 | 0.7 | 4.2 | 3.9 | 3.0 | 4.8 | 5.5 | 3.7 | 5.0 | 5.0 |
| 5 | 5 | 0.7 | 4.1 | 4.0 | 3.5 | 4.6 | 5.4 | 3.5 | 5.1 | 4.3 |
| 6 | 5 | 1.4 | 4.3 | 4.9 | 3.3 | 4.2 | 4.5 | 3.9 | 4.6 | 5.1 |
| 7 | 5 | 1.9 | 4.2 | 4.7 | 3.6 | 4.3 | 4.4 | 3.9 | 4.8 | 5.5 |
| 8 | 5 | 1.0 | 3.8 | 4.7 | 3.6 | 4.4 | 4.7 | 4.0 | 5.4 | 5.5 |
| 9 | 5 | 1.7 | 4.0 | 4.6 | 3.7 | 5.2 | 5.6 | 3.9 | 5.3 | 4.3 |
| 10 | 5 | 2.2 | 4.2 | 5.4 | 3.5 | 4.4 | 3.5 | 3.8 | 5.2 | 4.7 |

Inoculum level for S. typhimurium was 1.5 × 10⁶ CFU/ml.
Background APC before inoculation for cantaloupe 1 was 5.6 log CFU per cantaloupe; cantaloupe 2 was 5.8 log CFU per cantaloupe.
Following inoculation, S. typhimurium count for cantaloupe 1 was 5.2 log S. typhimurium per cantaloupe; cantaloupe 2 was 5.0 log S. typhimurium per cantaloupe.

### Example 42

Increasing the concentration of levulinic acid plus SDS and reducing the treatment time to 2 min resulted in greater reduction of microbes. The average S. typhimurium count, APC, and yeast and mold counts on cantaloupes treated with water only were 3.62, 6.36, and 4.45 log CFU, respectively, whereas, the average S. typhimurium, APC, and yeast and mold counts on cantaloupes treated with 2% levulinic acid plus 0.2% SDS were 1.02, 5.15, and 3.45 log CFU per cantaloupe, respectively (Table 44). Hence, the average reduction of S. typhimurium, APC, and yeast and mold counts was 2.6, 1.21, and 1.03 log CFU per cantaloupe. The average of S. typhimurium count on cantaloupes treated with 50 ppm acidified sodium chlorite was 3.43 log CFU per cantaloupe, for a reduction of 0.19 log Salmonella CFU per cantaloupe (Table 44).

Following treatment, the microbial counts of the treatment solutions were <log 0.7 log S. typhimurium/ml, and 1.2 log Y&M/ml for the 2.0% levulinic acid plus 0.2% SDS treatment solution; were 1.9 log S. typhimurium, and 3.7 log Y&M/ml for the 50 ppm acidified sodium chlorite solution; and were 4.0 log S. typhimurium/ml, and 3.6 log Y&M/ml for the water treatment solution.

TABLE 44
Microbial counts on cantaloupes treated at 21° C. for 5 mins in a 4-L tank

| | | Treated with 2.0% levulinic | | | Treated with 50 ppm acidified | | | | | |
|---|---|---|---|---|---|---|---|---|---|---|
| Canta- | | acid plus 0.2% SDS, pH 3.1 | | | sodium chlorite, pH 4.6 | | | Treated with water only | | |
| loupe | Time | Microbial counts (log CFU/whole cantaloupe) | | | | | | | | |
| No. | (min) | Salmonella | APC | Y&M | Salmonella | APC | Y&M | Salmonella | APC | Y&M |
| 1 | 2 | 1.5 | 6.6 | 4.3 | 3.7 | 6.4 | 4.8 | 3.8 | 6.4 | 4.0 |
| 2 | 2 | <0.7 | 5.8 | 3.7 | 3.6 | 6.1 | 4.9 | 3.8 | 6.1 | 5.6 |
| 3 | 2 | 1.0 | 4.6 | 3.2 | 3.3 | 5.3 | 5.0 | 3.5 | 6.7 | 4.8 |
| 4 | 2 | 1.2 | 5.9 | 3.8 | 3.5 | 7.0 | 5.1 | 3.4 | 6.3 | 4.4 |
| 5 | 2 | 1.7 | 4.2 | 3.4 | 3.7 | 6.0 | 4.8 | 3.6 | 6.4 | 4.1 |
| 6 | 2 | 1.0 | 5.2 | 2.8 | 3.3 | 6.3 | 5.1 | 3.7 | 6.8 | 4.8 |
| 7 | 2 | <0.7 | 5.2 | 2.8 | 3.2 | 6.2 | 5.1 | 3.3 | 5.9 | 4.1 |
| 8 | 2 | 0.7 | 4.8 | 2.5 | 3.5 | 6.8 | 4.5 | 3.7 | 6.2 | 4.0 |
| 9 | 2 | 0.7 | 4.4 | 4.0 | 3.1 | 6.7 | 4.0 | 3.7 | 6.7 | 4.6 |
| 10 | 2 | 1.2 | 4.8 | 4.0 | 3.4 | 6.6 | 4.5 | 3.7 | 6.1 | 4.4 |

Inoculum level for S. typhimurium is 1.5 × 10⁶ CFU/ml.
Background APC before inoculation of cantaloupe 1was 7.3 log CFU per cantaloupe; cantaloupe 2 was 6.4 log CFU per cantaloupe.
Following inoculation, S. typhimurium count for cantaloupe 1 was 5.0 log S. typhimurium per cantaloupe; cantaloupe 2 was 5.2 log S. typhimurium per cantaloupe.

### Example 43

TABLE 45
Inactivation of Salmonella in soap, levulinic acid and SDS

| | Adjusted to concentrations | | |
|---|---|---|---|
| | with levulinic acid and SDS | | |
| Soap^{a} | (v/v) | pH | |
| | Inoculation of Salmonella | | Salmonella enteritidis count |
| | enteritidis^{b} | | (log₁₀ CFU/ml), contact in |
| | | | soap for 1 min, 21° C. |
| Soap | Original | 7.0 | 7.7 |
| Soap | 0.5% levulinic acid + | 4.1 | 6.7 |
| | 0.05% SDS | | |
| Soap | 1.0% levulinic acid + | 3.7 | 5.1 |
| | 0.1% SDS | | |
| | Inoculation of S. | | S. typhimurium DT 104 count |
| | typhimurium DT 104^{c} | | (log₁₀ CFU/ml), contact in |
| | | | soap for 1 min, 21° C. |
| Soap | Original | 7.0 | 6.8 |
| Soap | 0.5% levulinic acid + | 4.15 | 6.3 |
| | 0.05% SDS 43 | | |
| Soap | 1.0% levulinic acid + | 3.95 | 6.0 |
| | 0.1% SDS | | |
| Soap | 2.0% levulinic acid + | 3.72 | <1.7^{d} |
| | 0.5% SDS | | |
| Soap | 3.0% levulinic acid + | 3.60 | <1.7 |
| | 0.3% SDS | | |

^{a}“Equate, hand soap with aloe vera” (compare to softsoap element soothing aloe vera), distributed by Wal-Mart Stores Inc., Bentonville, AR was used for this study. Ingredients contain: water, sodium laureth sulfate, sodium lauryl sulfate, cocamidopropylbetaine, sodium chloride, cocamide MEA, glycol stearate, benzyl alcohol, fragrance, polyquaternium-7, citric acid, tetrasodium EDTA, glycerin, aloe barbadensis leaf juice, hydrolyzed silk, glycol distearateglyceryl stearate.
^{b}a 5-strain mixture of Salmonella enteritidis was used.
^{c}a 5-strain mixture of S. typhimurium DT 104 was used.
^{d}the lowest detection level by direct plating method was 1.7 log₁₀ CFU/ml.

What is claimed:

1. An antimicrobial composition comprising:
a monoprotic organic acid comprising a carbon backbone of 3 to 13 carbons having the general structure of: 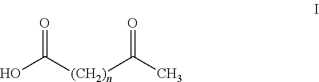
wherein n is an integer selected from 1 to 10, and wherein the concentration of the acid in said composition is about 0.2% to about 20% by weight per volume of solvent;
a surfactant, wherein the concentration of surfactant in said composition is about 0.05% to about 5% by weight per volume of solvent; and
an aqueous solvent,
wherein the antimicrobial composition is formulated to be effective in reducing the viability of a viral population, a bacterial population, a fungal population, or of any combination thereof.

2. The antimicrobial composition of claim 1, wherein the antimicrobial composition is formulated to be effective in reducing the viability of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

3. The antimicrobial composition of claim 1, wherein the surfactant is an anionic surfactant selected from the group consisting of: sodium dodecyl sulfate, sodium laureth sulfate, cetylpyridinium chloride, cetylpyridinium bromide, and benzalkonium chloride.

4. The antimicrobial composition of claim 1, further comprising a gelling agent, a foaming agent, a soap, a colorant, a fragrance, or any combination thereof.

5. The antimicrobial composition of claim 1, wherein the antimicrobial composition is formulated as a liquid; a foam having a cylinder foam test half-life of at least ten minutes, or a precursor thereof; a gel; or a solid or semi-solid soap.

6. The antimicrobial composition of claim 1, wherein the solvent is water or an alcohol:water mix, wherein the alcohol is selected from the group consisting of ethanol, propanol, isopropanol, butanol, propylene glycol, diethylene glycol, dipropylene glycol, or any mixture thereof.

7. The antimicrobial composition of claim 1, wherein the composition further comprises a cationic agent selected from the group consisting of: benzalkonium chloride, benzethonium chloride, triclocarban, tricolsan, chlorhexidine, and any combination thereof.

8. The antimicrobial composition of claim 1, wherein the composition is selected from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; about 5% levulinic acid and about 2% sodium dodecyl sulfate by weight per volume solvent.

9. The composition of claim 1, wherein the composition is deposited on or within a flexible support material.

10. The composition of claim 9, wherein the flexible support material is a cloth, a fabric, a paper, a natural fiber mesh, a synthetic fiber mesh, a combination natural and synthetic fiber mesh, a brush-like surface, or a porous fabric.

11. The composition of claim 1 substantially free of a solvent, wherein the pharmaceutically acceptable surfactant and the monoprotic organic acid are in a weight ratio of between about 1:200 to about 16.6:1.

12. A sanitizing wipe comprising a flexible support material and an antimicrobial composition absorbed thereon, wherein the antimicrobial composition comprises levulinic acid, sodium dodecyl sulfate, and a solvent, wherein the total concentration of the levulinic acid is about 0.2% to about 20% by weight per volume of solvent and the total concentration of the sodium dodecyl sulfate is about 0.05% to about 5% by weight per volume of solvent, and wherein the antimicrobial composition is formulated to be effective in reducing the viability of a microbial population.

13. The sanitizing wipe of claim 12, wherein the antimicrobial composition is formulated to be effective in reducing the viability of a viral population, a bacterial population, a fungal population, or of any combination thereof.

14. The antimicrobial composition of claim 13, wherein the antimicrobial composition is formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

15. The sanitizing wipe of claim 12, wherein the flexible support material has a surface positive charge thereon.

16. The sanitizing wipe of claim 12, wherein the solvent is water or an alcohol:water mix, wherein the alcohol is selected from the group consisting of ethanol, propanol, isopropanol, butanol, propylene glycol, diethylene glycol, dipropylene glycol, or any mixture thereof.

17. The sanitizing wipe of claim 12, wherein the composition further comprises a cationic agent selected from the group consisting of: benzalkonium chloride, benzethonium chloride, triclocarban, tricolsan, chlorhexidine, and any combination thereof.

18. The sanitizing wipe of claim 12, wherein the composition comprises from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume solvent; and about 5% levulinic acid by weight per volume solvent and about 2% sodium dodecyl sulfate by weight per volume solvent.

19. The sanitizing wipe of claim 12, further comprising a gelling agent, a foaming agent, a soap, a colorant, a fragrance, or any combination thereof.

20. The sanitizing wipe of claim 12, wherein the flexible support material is a cloth, a fabric, a paper, a natural fiber mesh, a synthetic fiber mesh, a combination natural and synthetic fiber mesh, a brush-like surface, or a porous fabric.

21. A method of reducing the viability of a microbial population, said method comprising contacting a microbial population with an antimicrobial composition comprising about 0.2% to about 20% by weight of levulinic acid per volume of solvent, about 0.05% to about 5% by weight of sodium dodecyl sulfate per volume of solvent, and an aqueous solvent, whereby the viability of the population of viruses is reduced.

22. The method of claim 21, wherein the microbial population is on a non-liquid surface.

23. The method of claim 21, wherein the microbial population is on a skin surface.

24. The method of claim 21, wherein the antimicrobial composition is formulated to be effective in reducing the viability of a viral population, a bacterial population, a fungal population, or of any combination thereof.

25. The method of claim 21, wherein the antimicrobial composition is formulated to be effective in reducing the viability of a population of a virus selected from the group consisting of: a respiratory syncytial virus (RSV), a coronavirus, an influenza virus, a measles virus, a Hepatitis B or C virus, a Herpes simplex virus, a norovirus, a sapovirus, an astrovirus, a rhinovirus, a rotavirus, an adenovirus, a Hepatitis E virus, and a Hepatitis A virus.

26. The method of claim 21, wherein the composition comprises from the group consisting of: about 0.25% to about 10% levulinic acid by weight per volume solvent and about 0.05% to about 5% sodium dodecyl sulfate by weight per volume solvent; about 0.5% levulinic acid by weight per volume solvent and about 0.5% sodium dodecyl sulfate by weight per volume; and about 5% levulinic acid by weight per volume solvent and about 2% sodium dodecyl sulfate by weight per volume solvent.

27. The method of claim 21, wherein the composition is disposed on a flexible support material.

28. The method of claim 26, wherein the flexible support material includes a positive ionic charge thereon.

29. The method of claim 21, wherein the antimicrobial composition applied to a viral population is formulated as a liquid wash, a spray, a foam, a paste, a cream, a gel, or a wipe.

30. The method of claim 21, wherein the antimicrobial composition is formulated to be effective in reducing the viability of a microbial population on a skin surface, wherein the microbial population is a viral population, a bacterial population, a fungal population, or any combination thereof, and wherein the antimicrobial composition is applied to the microbial population as a liquid wash, a spray, a foam, a paste, a cream, a gel, or a wipe.
